SEQUENCE LISTING (16 sequences)

SEQ ID NO: 1 - DNA, 20 bp, artificial sequence
  primer
catcgacttctacacgtcca

SEQ ID NO: 2 - DNA, 20 bp, artificial sequence
  primer
caaagtccttgagtcccaac

SEQ ID NO: 3 - DNA, 23 bp, artificial sequence
  primer
caagggccattgtgaatctccat

SEQ ID NO: 4 - DNA, 16 bp, artificial sequence
  primer
tgggtggcattgccaa

SEQ ID NO: 5 - DNA, 22 bp, artificial sequence
  primer
cgggaatggagaagtcactgct

SEQ ID NO: 6 - DNA, 24 bp, artificial sequence
  primer
caactctagcagaattggagggag

SEQ ID NO: 7 - DNA, 20 bp, artificial sequence
  primer
ggtctctgaccaggtgctgt

SEQ ID NO: 8 - DNA, 20 bp, artificial sequence
  primer
ggaatgcagttcagcatgag

SEQ ID NO: 9 - DNA, 21 bp, artificial sequence
  primer
cctttttttcagggaaagtgc

SEQ ID NO: 10 - DNA, 22 bp, artificial sequence
  primer
ccttaccatctccagaaactgc

SEQ ID NO: 11 - DNA, 22 bp, artificial sequence
  primer
tgcattctgagatccatacctt

SEQ ID NO: 12 - DNA, 22 bp, artificial sequence
  primer
ttcatcagacatatcccggttc

SEQ ID NO: 13 - DNA, 28 bp, artificial sequence
  probe
tctcccaacaaaccaatcccactttagg

SEQ ID NO: 14 - DNA, 21 bp, artificial sequence
  primer
acagagtgagaccccatcgca

SEQ ID NO: 15 - DNA, 22 bp, artificial sequence
  primer
tgtcttcctggggcctcagttt

SEQ ID NO: 16 - DNA, 29 bp, artificial sequence
  probe
taagtgaacaagaaattatctttattccc

(19) United States
(12) Patent Application Publication
(10) Pub. No.: US 2010/0092959 A1
(43) Pub. Date: Apr. 15, 2010

(54) Single nucleotide polymorphisms as genetic markers for childhood leukemia

(75) Inventors: Mehmet Tevfik Dorak, Hamilton, NJ (US); Esma Ucisik-Akkaya, Selden, NY (US); Thuy Ngoc Do, Hamilton, NJ (US); Charronne Davis, Mahwah, NJ (US); Brittany Morrison, Hamilton, NJ (US)

Correspondence Address: Medical Diagnostic Laboratories, LLC, 2439 Kuser Road, Hamilton, NJ, 08690, US

(73) Assignee: Medical Diagnostic Laboratories, LLC, Hamilton, NJ (US)

(21) Appl. No.: 12/455,520

(22) Filed: Jun. 3, 2009

Related U.S. Application Data
(60) Provisional application No. 61/130,797, filed on Jun. 3, 2008.
(60) Provisional application No. 61/130,798, filed on Jun. 3, 2008.
(60) Provisional application No. 61/132,692, filed on Jun. 20, 2008.
(60) Provisional application No. 61/208,376, filed on Feb. 23, 2009.

(51) Int. Cl. C12Q 1/68

(52) U.S. Cl. 435/6

(57) ABSTRACT

The present invention is directed to a panel of single nucleotide polymorphisms (SNPs) in specific genes that serve as biomarkers for sex-specific childhood leukemia risk. There is provided herein methods and reagents for assessing the specific SNPs in those genes. The method useful in applying these SNPs in predicting an increased risk or a decreased risk for childhood leukemia for males and females is also disclosed.

### CROSS-REFERENCE TO RELATED APPLICATIONS

This application claims the benefit under 35 U.S.C. §119(e) to U.S. Provisional Applications Nos. 61/130,797 filed Jun. 3, 2008, 61/130,798 filed Jun. 3, 2008, 61/132,692 filed Jun. 20, 2008 and 61/208,376 filed Feb. 23, 2009, the contents of which are incorporated by reference herein in their entirety.

### BACKGROUND OF THE INVENTION

Leukemia is a common type of cancer in childhood and represents a major killer disease of childhood only second to accidental deaths. Little is known about the causes of childhood leukemia and therefore precludes implementation of preventive measures (Linet et al, 2003). Ionizing radiation exposure, Down syndrome and rare genetic syndromes are established causes of childhood acute lymphoblastic leukemia (ALL), which is one of the most common leukemia type in childhood.

An important feature of childhood ALL is that it occurs more often in males. For each 100 females, 130 males will develop leukemia in childhood from birth to age 15 years (Linet et al, 2003). This observation suggests that males and females differ in their degree of susceptibility to develop childhood leukemia. Childhood leukemia may show sex-specificity.

The interest in the genetic determinants of leukemia risk was triggered by the demonstration by Lilly et al. that the major histocompatibility complex (MHC) genes may influence leukemia development in mice (Lilly et al., 1964). In this study, certain MHC genes accelerate the development of leukemia in mice. This finding was confirmed by others (See, e.g., Dorak M T, MHC and Leukemia; http://www.dorak.info/mhc/mhcleuk.html).

MHC is a collection of genes that are present in mammals (e.g., HLA complex in humans). Human HLA complex contains at least two hundred expressed genes that encode tissue antigens (HLA antigens) as well as other molecules including transcription factors, DNA repair molecules, apoptosis-related molecules. Many of these molecules may be involved in cancer susceptibility.

With respect to using HLA genes to predict cancer susceptibility, initial studies in humans failed to identify reliable risk markers. This is in part because of the unreliability of serological HLA typing methods. Expression levels of HLA gene are variable and many of them remain undetectable. Instead of serological approach, HLA typing using DNA is a more reliable tool. To this end, we have previously shown that an HLA gene (i.e., HLA-DRB4) associates in childhood leukemia acute lymphoblastic leukemia (Dorak et al. 1999a, 2002a). To the best of the present inventors' knowledge, HLA-DRB4 is one among few reported HLA gene markers for childhood leukemia. Notwithstanding its risk prediction value, HLA-DRB4 gene does not explain the entire childhood leukemia cases because not all patients possess DRB4 gene marker. Other reliable markers may be present within the HLA complex.

Earlier studies in the 1980's have identified HLA homozygosity (i.e., having two copies of the same antigen or allele/gene variant) as a risk marker. (Von Fliedner et al, 1980 & 1983; Carpentier et al, 1987). However, these studies utilized serological typing methods to type HLA antigens at the cell surface. The methods have low reliability in detecting homozygosity. It is because there may be a second allele that is undetectable by the methods. This would result in typing the sample as homozygote when it was actually heterozygote.

Recent studies suggest some benefits of heterozygosity (Campbell et al, 2007). There is no information regarding HLA complex heterozygosity, let alone its role in cancer development. The role of heterozygote advantage for childhood leukemia in human HLA is presently unknown. In mice, heterozygote advantage at the MHC (H-2 complex) was first recognized as protective against infectious diseases (Doherty & Zinkernagel, 1975). It is speculated that heterozygosity at the MHC in mice can enhance immunological surveillance. The discoverers of that effect were awarded the Nobel Prize in Physiology or Medicine in 1996. Whether the mice observation relating MHC (H-2 complex) in infectious diseases may similarly apply in cancers of human is presently unknown.

Single nucleotide polymorphism (SNP) is a common form of genetic polymorphisms. SNPs may influence gene functions and modifies an individual's susceptibility to diseases. Almost any diseases have a genetic component in its etiology and most are being unraveled in genetic association studies. In some instances, a single SNP may be sufficient to confer susceptibility, while in others multiple SNPs may act jointly to influence disease susceptibility. An estimated 20 million SNPs are present in human genome. This astronomical number precludes individual screening of each and every one because of the huge work and cost.

To the best of the present inventors' knowledge, there are no reliable genetic markers for childhood leukemia risk that has clinical utility. There is no information relating to any SNPs that may be of any predictive value in childhood leukemia, let alone that they are present in HLA complex, iron regulatory gene, or cytokine genes.

Accordingly, there is a continuing need for a genetic marker that can reliably predict childhood leukemia. The need for a reliable SNP biomarker for childhood leukemia may have practical utility in neonatology clinics. Such SNP biomarker may provide useful information regarding whether or not to store the newborn's own cord blood used for treatment if leukemia may develop later in childhood. The SNP biomarker may also provide useful information throughout the entire childhood period in informing patients' families for possible leukemia development. The panel of SNP disclosed in this application can be used to assess the risk for childhood leukemia, even in pre-implantation genetic testing in an IVF clinic as well as during the entire prenatal period by obstetricians. The SNP panel enables one to assess the risk for a prospective offspring of a family if they are highly concerned, such as having had another child with childhood leukemia or a family history of childhood leukemia.

### BRIEF SUMMARY OF THE INVENTION

The present invention is directed to novel single nucleotide polymorphisms (SNPs) in specific genes and that the presence of one or more of these SNPs is a highly specific marker for childhood leukemia in females or males.

In one aspect, the present invention provides in female the specific genes that include HLA gene, iron regulatory gene, cytokine gene, and other related genes that encompass EGF rs444-4903, EDN1 rs5370, VEGFA rs1570360, and TP53 rs1042522. In another aspect, the present invention provides in male the specific genes that include HLA gene, iron regulatory gene, cytokine gene, and other related genes that encompass ACP1 rs12714402, and TP53 rs1042522.

Accordingly, the present invention provides methods for detecting childhood leukemia in individuals. The methods include detecting at least one SNP in the specific genes.

In one aspect, the present invention provides a method of determining a risk for childhood leukemia in a female, comprising the steps of:
(a) obtaining a biological sample from a female;
(b) isolating nucleic acids from said biological sample; and
(c) performing polymerase chain reaction (PCR) on said isolated nucleic acids to determine the presence of a SNP present in a gene selected from the group consisting of a HLA gene, iron regulatory gene, and cytokine gene, wherein:
   (i) at least one SNP selected from the group consisting of BMP6 rs17557, UBD rs2534790, HLA-G rs1736939, HLA-G rs1704, ZNRD1 rs9261269, DDR1 rs1264328, DDR1 rs1264323, DDR1 rs1049623, HLA-C rs9264942, SKIV2L rs419788, HLA-DRA rs3135388, DAXX rs2073524, DAXX rs1059231, and DAXX rs2239839 that is present in said HLA gene, or
   (ii) at least one SNP selected from the group consisting of STEAP3 rs865688, SLC40A1 rs1439812, SLC40A1 rs1439812, HFE rs807212, TFR2 rs10247962, LCN2 rs878400, SLC11A2 rs224589, HMOX1 rs2071748, and HMOX1 rs5755709 that is present in said iron regulatory gene, or
   (iii) at least one SNP selected from the group consisting of IL6 rs1800797 and IL10 rs1800872 that is present in said cytokine gene, and
wherein the presence of said SNP present in said gene is indicative of a risk for childhood leukemia in said female.

Accordingly, the presence of UBD rs2534790, SKIV2L rs419788, HLA-DRA rs3135388, DAXX rs2073524, DAXX rs1059231, DAXX rs2239839, SLC40A1 rs1439812, TFR2 rs10247962, or IL6 rs1800797 is indicative for an increased risk for childhood leukemia in female children.

Accordingly, the presence of BMP6 rs17557, HLA-G rs1736939, HLA-G rs1704, ZNRD1 rs9261269, DDR1 rs1264328, DDR1 rs1264323, DDR1 rs1049623, HLA-C rs9264942, STEAP3 rs865688, HFE rs807212, LCN2 rs878400, SLC11A2 rs224589, HMOX1 rs2071748, HMOX1 rs5755709, IL10 rs1800872, or SLC40A1 rs1439812 is indicative for a decreased risk for childhood leukemia in female children.

Preferably, SNP may include a combination of HLA-G rs1736939 and HLA-G rs1704, or a combination of DDR1 rs1264328, DDR1 rs1264323, and DDR1 rs1049623, wherein the presence of said combination is indicative of a decreased risk for childhood leukemia.

Preferably, SNP may include a combination of DAXX rs2073524, DAXX rs1059231, and DAXX rs2239839 in HLA gene, the presence of said combination is an increased risk for childhood leukemia.

In another aspect, the present invention provides SNPs that may include a combination of at least 4 SNPs selected from the group consisting of DRB, DAXX haplotype, EDN1 rs5370, HMOX1 rs2071748, TFR2 rs10247962, TP53 rs1042522, and IL10 rs1800872. The presence of this combination of at least 4 SNPs is indicative of an increased risk of childhood leukemia.

Preferably, SNP may include is a combination of at least 5 SNPs selected from the group consisting of DRB, DAXX haplotype, EDN1 rs5370, HMOX1 rs2071748, TFR2 rs10247962, TP53 rs1042522, and IL10 rs1800872. The presence of this combination of at least 5 SNPs is also indicative of an increased risk of childhood leukemia.

In another aspect, the present invention provides a method of determining a risk for childhood leukemia in a male, comprising the steps of:
(a) obtaining a biological sample from a male;
(b) isolating nucleic acids from said biological sample; and
(c) performing polymerase chain reaction (PCR) on said isolated nucleic acids to determine the presence of a SNP present in a gene selected from the group consisting of a HLA gene, iron regulatory gene, and cytokine gene, wherein:
   (i) at least one SNP selected from the group consisting of NFKB1 rs4648022, MICA rs1051792, MICA STR allele 185 bp (A5.1), BAT3 rs2077102, HSPA1B rs1061581, BTNL2 rs9268480, HLA-DRA rs7192, HLA-DQA1 rs1142316, NOTCH4 rs3096702, HLA-DRB1-DQA1 rs2395225, and HLA-DRB1-DQA1 rs9271586 that is present in said HLA gene; or
   (ii) at least one SNP selected from the group consisting of TF rs1049296, TF rs8649, TF rs1130459, TF rs4481157, LTF rs1042073, HFE rs807212, SLC39A14 rs11136002, SLC39A4 rs2272662, LCN2 rs878400, TMPRSS6 rs733655, and TMPRSS6 rs855791 that is present in said iron regulatory gene; or
   (iii) at least one SNP selected from the group consisting of ILK, rs1800872, PKR rs2270414, PKR rs12712526, PKR rs2254958, CTLA4 rs231775, IRF4 rs12203592, NKG2D rs1049174, NKG2D rs2617160, NKG2D rs2734565, NKG2D rs2617170, NKG2D rs2617171, NKG2D rs1841958, NKG2D rs1983526, and IFNG rs2069727 that is present in said cytokine gene, and
wherein the presence of said SNP present in said gene is indicative of a risk for childhood leukemia in said male.

Accordingly, the presence of rs1051792, MICA STR allele 185 bp (A5.1), HSPA1B rs1061581, BTNL2 rs9268480, HLA-DRA rs7192, HLA-DQA1 rs1142316, NOTCH4 rs3096702, HLA-DRB1-DQA1 rs2395225, and HLA-DRB1-DQA1 rs9271586, SLC39A4 rs2272662, TMPRSS6 rs733655, CTLA4 rs231775, IRF4 rs12203592, NKG2D rs1049174, NKG2D rs2617160, NKG2D rs2734565, NKG2D rs2617170, NKG2D rs2617171, NKG2D rs1841958, or NKG2D rs1983526 is indicative for an increased risk for childhood leukemia in male children.

Accordingly, the presence of NFKB1 rs4648022, BAT3 rs2077102, HSPA1B rs1061581, TF rs1049296, TF rs8649, TF rs1130459, TF rs4481157, LTF rs1042073, HFE rs807212, SLC39A14 rs11136002, TMPRSS6 rs855791, IL10 rs1800872, PKR rs2270414, PKR rs12712526, PKR rs2254958, IFNG rs2069727, or LCN2 rs878400 is indicative for a decreased risk for childhood leukemia in male children.

Preferably, SNP may include a combination of MICA rs1051792 and MICA STR allele 185 bp (A5.1), a combination of HSPA1B rs1061581, BTNL2 rs9268480, and HLA-DRA rs7192, a combination of HSPA1B rs1061581, HLA-DRA rs7192, and HLA-DQA1 rs1142316, or a combination of HLA-DRB1-BQA1 rs2395225 and HLA-DRB1-DQA1 rs9271586 in HLA gene, the presence of said combinations is indicative of an increased risk for childhood leukemia in male children.

Preferably, SNP may include a combination of TF rs1049296, TF rs8649, TF rs1130459, and TF rs4481157, or a combination of PKR rs2270414, PKR rs12712526, and PKR rs2254958 in iron regulatory gene, the presence of said combination is indicative of a decreased risk for childhood leukemia in male children.

Preferably, SNP may include a combination of NKG2D rs1049174, NKG2D rs2617160, NKG2D rs2734565, NKG2D rs2617170, NKG2D rs2617171, NKG2D rs1841958, and NKG2D rs1983526 in cytokine gene, the presence of said combination is indicative of an increased risk for childhood leukemia in male children.

In another aspect, the present invention provides SNPs that may include a combination of at least 4 SNPs selected from the group consisting of DRB1 region, HSPA1B rs1061581, MICA haplotype, HFE rs807212, TMPRSS6 rs733655, LTF rs1042073, and PKR haplotype, wherein the presence of said combination of at least 4 SNPs is indicative of an increased risk for childhood leukemia in male children.

Preferably, the SNP is a combination of at least 5 SNPs selected from the group consisting of DRB1 region, HSPA1B rs1061581, MICA haplotype, HFE rs807212, TMPRSS6 rs733655, LTF rs1042073, and PKR haplotype, wherein the presence of said combination of at least 5 SNPs is indicative of an increased risk for childhood leukemia in male children.

In another aspect, the present invention provides a SNP that serves as a reliable predictor for childhood leukemia. Exemplary childhood leukemia includes childhood acute lymphoblastic leukemia (ALL), acute myeloblastic leukemia (AML), and the like.

In another aspect, the biological sample may be any suitable sample from an individual, including, but not limited to, whole blood, a buccal mucosal swab, skin, hair, tissue and the like. Preferably, blood may be umbilical cord blood.

In another aspect, nucleic acids are genomic DNA and may be isolated using phenol-chloroform, salting out, silica membrane adsorption, magnetic beads, and the like. Preferably, isolating step is performed using phenol-chloroform.

In another aspect, the detecting step may be performed by a polymerase chain reaction (PCR). Exemplary PCR methods may include, for example, TaqMan allelic discrimination assay or PCR-restriction fragment length polymorphism assay.

### BRIEF DESCRIPTION OF THE DRAWINGS

FIG. 1 depicts the genomic location of the single nucleotide polymorphisms (SNPs) evaluated for their values to predict sex-specific childhood leukemia risk by genotyping cases who have developed childhood acute lymphoblastic leukemia by age 15 years and healthy newborns as controls.

FIG. 2 depicts the individual and additive predictive power of the independent predictive subset of single nucleotide polymorphisms (SNPs) as biomarkers for childhood acute lymphoblastic leukemia in females.

FIG. 3 depicts the individual and additive predictive power of the independent predictive subset of single nucleotide polymorphisms (SNPs) as biomarkers for childhood acute lymphoblastic leukemia in males.

### DETAILED DESCRIPTION OF THE INVENTION

The present inventors cured the prior art deficiency and used a DNA-based approach to identify genetic markers in predicting sex-specific childhood leukemia risk. The present invention provides genetic markers of leukemia risk. The present invention provides comparison of genotype frequencies that provide clues for the involvement of genes in childhood leukemia risk. Selected gene candidate in biologically plausible targets such as HLA complex, iron-regulatory gene, immune surveillance system-related genes (NKG2D/KLRK1 and cytokines) and other cancer related genes were genotyped in healthy newborns and children who developed childhood leukemia.

The present inventors discovered that specific single nucleotide polymorphisms (SNPs) in these genes represent good predictors for sex-specific childhood leukemia risk, and that the males and females differ in their genetic susceptibility to childhood leukemia.

### DEFINITIONS

Various terms used throughout this specification shall have the definitions set forth herein.

The term “polymorphism” refers to the occurrence of two or more alternative genomic sequences or alleles between or among different genomes or individuals.

The term “polymorphic” refers to the condition in which two or more variants of a specific genomic sequence found in a population.

The term “polymorphic site” is the locus at which the variation occurs. A polymorphic site generally has at least two alleles, each occurring at a significant frequency in a selected population. A polymorphic locus may be as small as one base pair, in which case it is referred to as single nucleotide polymorphism (SNP). The first identified allelic form is arbitrarily designated as the reference, wild-type, common or major form, and other allelic forms are designated as alternative, minor, rare or variant alleles.

The term “genotype” refers to a description of the alleles of a gene contained in an individual or sample.

The term “single nucleotide polymorphism” (“SNP”) refers to a site of one nucleotide that varies between alleles.

The term “functional SNPs” refers to those SNPs that produce alterations in gene expression or in the expression or function of a gene product, and therefore are most predictive of a possible clinical phenotype. The alterations in gene function caused by functional SNPs may include changes in the encoded polypeptides, changes in mRNA stability, binding of transcriptional and translation factors to the DNA or RNA, and the like.

The term “HLA gene” refers to human leukocyte antigen genes (i.e., MHC genes that have known immunological functions) located within the HLA complex that is situated on chromosome 6p21.3. In humans, HLA complex is a 3.6-Mb (3,600,000 bp) region on chromosome 6 that contains 140 genes between flanking genetic markers MOG and COL11A2. There are non-HLA genes (e.g., UBD, ZNRD1, SKIV2L, DAXX, BAT3, HSPA1B, BTNL2, NOTCH4, MICA, DDR1, BMP6) that are also situated within the HLA complex. For purposes of this application, the term “HLA gene” encompasses all the human leukocyte antigen genes within the HLA complex as well as specific non-HLA-genes (genes as recited herein) that are situated within the same HLA complex.

The term “iron regulatory gene” refers to genes that regulate iron level in a human body. Exemplary iron regulatory genes include, but not limited to, STEAP3, SLC40A1, HFE, TF, TFR2, TFRC, LCN2, SLC11A2, HMOX1, LTF, SLC39A14, SCL39A4, TMPRSS6, and the like).

The term “cytokine gene” refers generally to genes of immune surveillance system. Cytokine genes encode cytokine proteins. For purposes of this application, cytokine gene encompasses IL6, IL10, IFNG, LIF as well as specific genes within the immune surveillance system genes (such as CTLA4, NKG2D, IRF4, and PKR).

The term “cancer-related gene” refers to specific genes of TP53, MDM2, EGF, VEGFA, EDN1, ACP1, and the like that are generally related cancer development processes such as DNA repair, apoptosis, angiogenesis, and cell proliferation.

The term “short tandem repeat” (STR) polymorphism refers to genomic sequences of 2 to 5 nucleotide long repeated up to 50 times such as a TA dinucleotide repeat polymorphism. STR polymorphism is also called microsatellite polymorphism. The variable number of repeats in each individual creates the polymorphism. They may occur in thousands of locations in the human genome.

The term “haplotype” refers to a string of SNP alleles represented consecutively on the same chromosome. A haplotype for example may consist of all wildtype alleles of three SNPs or different alleles of each one.

The term “oligonucleotide” is used interchangeable with “primer” or “polynucleotide.”

The term “primer” refers to an oligonucleotide that acts as a point of initiation of DNA synthesis in a PCR reaction. A primer is usually about 15 to about 35 nucleotides in length and hybridizes to a region complementary to the target sequence.

The term “probe” refers to an oligonucleotide that hybridizes to a target nucleic acid in a PCR reaction. Target sequence refers to a region of nucleic acid that is to be analyzed and comprises the polymorphic site of interest.

The term “TaqMan allelic discrimination assay” (also known as the 5′ nuclease PCR assay) is a technology that exploits the 5′-3′ nuclease activity of Taq DNA polymerase to allow direct detection of the polymorphic nucleotides by the release of a fluorescent reporter as a result of PCR. The TaqMan allelic discrimination assay permits discrimination between the alleles of a two-allele system. It represents a sensitive and rapid means of genotyping SNPs.

The term “PCR-RFLP” refers to polymerase chain reaction-restriction fragment length polymorphism. PCR-RFLP is technique to detect a variation in the DNA sequence of a genome by breaking the DNA into pieces with restriction enzymes and analyzing the size of the resulting fragments by gel electrophoresis. PCR-RFLP is one type of genotyping for detecting SNP by visualization of fragments on a gel following restriction endonuclease digestion of the PCR product.

The term “high-resolution melting” (HRM) analysis refers to a genotyping method based on melting temperature differences of genomic fragments carrying different alleles of a polymorphism. First, a DNA sample is obtained from an individual, a specific fragment is amplified by PCR and is heated in a specialized instrument to detect the presence of allelic differences. The genotype is determined by observing melting curve (also known as dissociation curve) profile of each sample. In this method, no fluorescent probe or biochemical manipulation are used.

The term “odds ratio” (OR) refers to the approximate ratio of the frequency of the disease in individuals having a particular marker (allele or polymorphism) to the frequency of the disease in individuals without the marker (allele or polymorphism).

The term “an increased risk for childhood leukemia” refers to a situation where the probability of a healthy newborn carrying a certain marker to develop leukemia is greater compared with another healthy newborn that does not possess the same marker.

The term “a decreased risk for childhood leukemia” refers to a situation where the probability of a healthy newborn carrying a certain marker to develop leukemia is lesser compared with another healthy newborn that does not possess the same marker. For purposes of this application, an odds ratio of >1.5 with a statistical significance of P≦0.05 indicates an increased risk, and an odds ratio of >1.95 (i.e., more than two-fold increased risk) and a statistical significance of P≦0.05 indicate a strong increased risk. On the other hand, an odds ratio of <0.70 and a statistical significance of P≦0.05 indicates a reduced risk, and an odds ratio of <0.55 (i.e., more than two-fold decreased risk) and a statistical significance of P<0.05 indicate a strong reduced risk.

The term “95% confidence interval” (or “95% CI”) refers to the range of values surrounding the odds ratio within which the true value is believed to lie with 95% certainty.

The term “heterozygote advantage” refers to protection from a condition conferred by a heterozygous genotype. The classic example is better protection of individuals who are heterozygous at immune system genes (such as HLA genes) from infectious diseases.

The term “Hardy-Weinberg equilibrium” refers to a principle that allele and genotype frequencies in a population remain constant; that is, they are in equilibrium-from generation to generation unless specific disturbing influences are introduced. Those disturbing influences include non-random mating, mutations, selection, limited population size, random genetic drift and gene flow. In the simplest case of a single locus with two alleles: one allele is denoted “A” and the other “a” and their frequencies are denoted by p and q; freq(A)=p; freq(a)=q; p+q=1. According to the Hardy-Weinberg principle, when the population is in equilibrium, then we will have freq(AA)=p² for the AA homozygotes in the population, freq(aa)=q² for the aa homozygotes, and freq(Aa)=2pq for the heterozygotes.

The term “haplotype tagging SNPs” (htSNPs) refers to a subset of SNPs in each gene that provides sufficient information about genetic variation in a gene as genotyping all of the SNPs in a gene. They basically represent other SNPs in their vicinity and make the others redundant in terms of providing additional information about genetic variation.

The term “linkage disequilibrium” refers to the non-random association in population genetics of alleles at two or more loci. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium.

The term “multivariable analysis” refers to an analysis used to assess the independent contribution of each of the multiple risk markers that contribute to a disease condition. That is, multivariable analysis helps to determine the most informative minimal set of independent (uncorrelated) multiple risk markers (variables). In situations where two SNPs from the same gene show statistically significant association, but when tested together in a multivariable analysis, if they are correlated, one of them loses significance and the other one is called an independent marker. The one that is no longer significantly associated is still useful in estimation of the risk in the absence of any other marker, but its association is only due to its relationship with a stronger marker. Since human diseases are often influenced by multiple genes, it is usual to find associations with many SNPs from many genes. In this case, a multivariable analysis is used to eliminate any redundant markers.

The term “adjusted odds ratio” refers to an odds ratio that is adjusted with another factor (e.g., age). When all independent risk markers are analyzed together in a multivariable analysis, the odds ratio for each marker may be slightly different from the odds ratios obtained from analysis of each SNP on its own. These new odds ratios are called adjusted odds ratios. Since no SNP acts on its own in reality, these adjusted odds ratios represent a more realistic estimate of the risk. These are odds ratios calculated by statistical algorithms that take into account individual contributions of any other risk marker (variable) included in the multivariable analysis.

The present invention provides SNPs associated with childhood leukemia, methods and reagents for the detection of the SNPs disclosed herein, uses of these SNPs for the development of detection reagents, and assays or kits that utilize such reagents. The childhood leukemia-associated SNPs disclosed herein are useful for diagnosing, screening for, and evaluating predisposition to childhood leukemia in humans.

Accordingly, the present inventors have established a highly specific correlation for particular SNP genotypes in various genes in male and female children. The high specificity of these SNP correlations with development of childhood leukemia provides a reliable and specific prediction that the presence of a specific SNP is a good predictor for occurrence of childhood leukemia. Accordingly, the present invention provides, inter alia, useful tools for physicians to make proper diagnosis and risk prediction that would predict a lower incidence or reduced risk for childhood leukemia. Alternatively, the present invention also provides useful tools for physicians to make proper diagnosis and risk predication that would predict a higher incidence or increased risk for childhood leukemia. SNP genotyping of an individual (such as a child) enables doctors to select an appropriate medication, dosage regimes, and duration of treatment that will be effective based on an individual's SNP genotype.

In particular, the present inventors have discovered a panel of SNPs in male children that are highly specific and bear a high correlation with the development of childhood leukemia. In male, the specific genes include HLA gene, iron regulatory gene, cytokine gene, and other related genes that encompass ACP1 rs12714402, and TP53 rs1042522.

In particular, the present inventors also discovered a panel of SNPs in female children that are highly specific and similarly bear a high correlation with the development of childhood leukemia. In female, the specific genes include HLA gene, iron regulatory gene, cytokine gene, and other related genes that encompass EGF rs444-4903, EDN1 rs5370, VEGFA rs1570360, and TP53 rs1042522.

In one embodiment, the present invention provides a panel of SNPs that exhibit associations with sex-specific risk for childhood leukemia development. The SNPs identified are present in specific candidate genes. In another embodiment, the present invention provides a method of using genotyping approach to identify a panel of SNPs listed in Tables 3-6 out of all the 311 SNPs listed in Table 1. Provided in Tables 3-6 are the panels of SNPs that have predictive values in either an increased risk or decreased risk for childhood leukemia for female (Tables 3-4) and male (Tables 5-6). When an odds ratio is >1.5 with a statistical significance of P≦0.05, this indicates an increased risk. When an odds ratio is >1.95 with a statistical significance of P≦0.05, this indicates a strong increased risk. On the other hand, when an odds ratio is <0.70 with a statistical significance of P≦0.05, this indicates a reduced risk. And when an odds ratio is <0.55 with a statistical significance of P≦0.05, this indicates a strong reduced risk

In accordance with the present invention, one of a skilled artisan understands that SNPs have two alternative alleles, each corresponds to a nucleotide that may exist in the chromosome. Thus, a SNP is characterized by two nucleotides out of four (A, C, G, T). An example would be that a SNP has either allele C or allele T at a given position on each chromosome. This is shown as C>T or C/T. The more commonly occurring allele is shown first (in this case, it is C) and called the major, common or wild-type allele. The alternative allele that occurs less commonly instead of the common allele (in this case, it is T) is called minor, rare or variant allele. To avoid confusion, in this patent application, we adopted to use wild-type and variant allele to define the common and rare alleles. Since humans are diploid organisms meaning that each chromosome occurs in two copies, each individual has two alleles at a SNP. These alleles may be two copies of the same allele (CC or TT) or they may be different ones (CT). The CC, CT and TT are called genotypes. Among these CC and TT are characterized by having two copies of the same allele and are called homozygous genotypes. The genotype CT has different alleles on each chromosome and is a heterozygous genotype. Individuals bearing homozygote or heterozygote genotypes are called homozygote and heterozygote, respectively.

Providing a biological sample may include for example, collecting a sample from a child (male or female), and isolating nucleic acids (e.g., genomic) from cells of the sample. The biological sample collected from the children may be any suitable biological sample as would be apparent to those skilled in the art, and may include for example, blood, buccal mucosal cells, skin, hair and tissue and the like. Preferably, blood may include umbilical cord blood or venous blood.

The present inventors discovered that by examining genotype frequencies of polymorphisms in cases with childhood leukemia and healthy newborn controls, clues may be obtained as to which genes are involved in development of childhood leukemia. This can be achieved by comparing genotype frequencies in cases and controls and for each sex (i.e., males and females), separately. In one embodiment, the present invention provides a method of using genotype data rather than sequence data, SNPs are identified to support the findings in the association study.

HWE tests check the agreement between observed genotype frequencies and expected frequencies calculated from observed allele frequencies. A perfect agreement is expected when several assumptions are met. One of the assumptions is the absence of selection. A statistically significant result in the goodness-of-fit test examining the agreement suggests disequilibrium. The cause for this is change in genotype distribution in the population is usually selection. In practice, however, the most common cause for Hardy-Weinberg disequilibrium is genotyping errors. In this application, only those SNPs whose genotype distributions were in Hardy-Weinberg equilibrium were used in prediction of childhood leukemia risk.

In accordance with the present invention, there is disclosed an optimal approach that utilizes genotyping to provide direct evidence for increased risk for developing childhood leukemia. In this approach, if a genotype has a deleterious effect on the development of leukemia on a newborn child, cases with leukemia will have an increased frequency for that genotype compared with newborns.

In one embodiment, the present invention provides a method of utilizing an individual SNP to predict susceptibility to childhood leukemia. In accordance with the present invention, the assessing techniques to determine the presence of a SNP are known in the field of molecular genetics. Further, many of the methods involve amplification of nucleic acids. (See, PCR Technology: Principles and Applications for DNA Amplification (Ed. H. A. Erlich, Freeman Press, NY, N.Y., 1992), and Current Protocols in Molecular Biology, Ausubel, 1999).

It is understood that there are many methodologies currently existing for the detection of single nucleotide polymorphisms (SNPs) that are present in genomic DNA. SNPs are DNA point mutations or insertions/deletions that are present at measurable frequencies in the population. SNPs are the most common variations in the genome. SNPs occur at defined positions within genomes and can be used for gene mapping, defining population structure, and performing functional studies. Sometimes, SNPs are useful as markers because many known genetic diseases are caused by point mutations and insertions/deletions.

In one embodiment, the detection of the presence of a SNP in a particular gene is genotyping. According to non-limiting example embodiments, the detecting step may be performed by real-time PCR, conventional PCR followed by pyrosequencing, single-base extension and the like. These PCR methodologies are well within the knowledge of a skilled artisan.

Provided herein is optimal real-time PCR in detecting SNPs that are present in specific gene candidates as recited in the application. In example embodiments an analytical detection, such as a fluorescence detection method may be provided, in conjunction with PCR based on specific primers directed at SNP regions within the selective gene candidates. In such embodiments, SNP detection using real-time amplification relies on the ability to detect amplified segments of nucleic acid as they are during the amplification reaction.

Presently, three basic real-time SNP detection methodologies exist: (i) increased fluorescence of double strand DNA specific dye binding, (ii) decreased quenching of fluorescence during amplification, and (iii) increased fluorescence energy transfer during amplification. All these techniques are non-gel based and each detection methodology may be conveniently optimized to detect SNPs.

According to non-limiting example embodiments, real-time PCR may be performed using exonuclease primers (TaqMan® probes). In such embodiments, the primers utilize the 5′ exonuclease activity of thermostable polymerases such as Taq to cleave dual-labeled probes present in the amplification reaction (See, e.g., Wittwer, C. et al. Biotechniques 22:130-138, 1997). While complementary to the PCR product, the primer probes used in this assay are distinct from the PCR primer and are dually-labeled with both a molecule capable of fluorescence and a molecule capable of quenching fluorescence. When the probes are intact, intramolecular quenching of the fluorescent signal within the DNA probe leads to little signal. When the fluorescent molecule is liberated by the exonuclease activity of Taq during amplification, the quenching is greatly reduced leading to increased fluorescent signal. Non-limiting example fluorescent probes include 6-carboxy-floruescein moiety and the like. Exemplary quenchers include Black Hole Quencher 1 moiety and the like.

Detection of SNPs in specific gene candidates may be performed using real-time PCR, based on the use of intramolecular quenching of a fluorescent molecule by use of a tethered quenching moiety. Thus, according to example embodiments, real-time PCR methods may include the use of molecular beacon technology. The molecular beacon technology utilizes hairpin-shaped molecules with an internally-quenched fluorophore whose fluorescence is restored by binding to a DNA target of interest (See, e.g., Kramer, R. et al. Nat. Biotechnol. 14:303-308, 1996). Increased binding of the molecular beacon probe to the accumulating PCR product can be used to specifically detect SNPs present in genomic DNA.

Methods provided herein may include the use any suitable primer set(s) capable of detecting SNPs. The selection of a suitable primer set may be determined by those skilled in the art, in view of this disclosure. By way of non-limiting example, the primers provided in the “Experimental Protocols”, infra, may be used in detection of one or more SNPs.

Real-time PCR methods may also include the use of one or more hybridization probes, which may also be determined by those skilled in the art, in view of this disclosure. By way of non-limiting example, such hybridization probes may include one or more of those provided in the “Experimental Protocols.” Exemplary probes such as the HEX channel and/or FAM channel probes, as understood by one skilled in the art.

According to example embodiments, probes and primers may be conveniently selected e.g., using an in silico analysis using primer design software and cross-referencing against the available nucleotide database of genes and genomes deposited at the National Center for Biotechnology Information (NCBI). Some additional guidelines may be used for selection of primers and/or probes. For example the primers and probes may be selected such that they are close together, but not overlapping. The primers may have the same (or close T_{M}) (e.g. between 58° C. and 60° C.). The T_{M} of the probe may be approximately 10° C. higher than that selected for the T_{M} of the primers. The length of the probes and primers should be between about 17 and 39 base pairs, etc. These and other guidelines may be useful to those skilled in the art in selecting appropriate primers and/or probes.

One of the many suitable genotyping procedures is the TaqMan allelic discrimination assay. In this assay, one may utilize an oligonucleotide probe labeled with a fluorescent reporter dye at the 5′ end of the probe and a quencher dye at the 3′ end of the probe. The proximity of the quencher to the intact probe maintains a low fluorescence for the reporter. During the PCR reaction, the 5′ nuclease activity of DNA polymerase cleaves the probe, and separates the dye and quencher. Thus resulting in an increase in fluorescence of the reporter. Accumulation of PCR product is detected directly by monitoring the increase in fluorescence of the reporter dye. The 5′ nuclease activity of DNA polymerase cleaves the probe between the reporter and the quencher only if the probe hybridizes to the target and is amplified during PCR. The probe is designed to straddle a target SNP position and hybridize to the nucleic acid molecule only if a particular SNP allele is present.

Genotyping is performed using oligonucleotide primers and probes. Oligonucleotides may be synthesized and prepared by any suitable methods (such as chemical synthesis), which are known in the art. Oligonucleotides may also be conveniently available through commercial sources. One of the skilled artisans would easily optimize and identify primers flanking the gene of interest in a PCR reaction. Commercially available primers may be used to amplify a particular gene of interest for a particular SNP. A number of computer programs (e.g., Primer-Express) is readily available to design optimal primer/probe sets. It will be apparent to one of skill in the art that the primers and probes based on the nucleic acid information provided (or publicly available with accession numbers) can be prepared accordingly.

The labeling of probes is known in the art. The labeled probes are used to hybridize within the amplified region during the amplification region. The probes are modified so as to avoid them from acting as primers for amplification. The detection probe is labeled with two fluorescent dyes, one capable of quenching the fluorescence of the other dye. One dye is attached to the 5′ terminus of the probe and the other is attached to an internal site, so that quenching occurs when the probe is in a non-hybridized state.

As appreciated by one of skill in the art, other suitable genotyping assays may be used in the present invention. This includes hybridization using allele-specific oligonucleotides, primer extension, allele-specific ligation, sequencing, electrophoretic separation techniques, and the like. Exemplary assays include 5′ nuclease assays, molecular beacon allele-specific oligonucleotide assays, and SNP scoring by real-time pyrophosphate sequences.

Determination of the presence of a particular SNP is typically performed by analyzing a nucleic acid sample present in a biological sample obtained from an individual. Biological sample is derived from a child whose risk to develop leukemia is being assessed. DNA can be obtained from peripheral blood cells (including heel-prick), buccal swab cells, cells in mouth wash or any other cell or tissue. The nucleic acid sample comprises genomic DNA, mRNA or isolated DNA. The nucleic acid may be isolated from blood samples, cells or tissues. Protocols for isolation of nucleic acid are known. Exemplary DNA isolation protocols include phenol-chloroform extraction, salting out, silica membrane adsorption, magnetic beads, and the like. Preferably, DNA is isolated using phenol-chloroform.

PCR-RFLP represents an alternative genotyping method used in the invention. PCR-RFLP can yield unambiguous results provided that there is a suitable endonuclease that will cut the amplified PCR product containing a SNP if it contains one of the alternative nucleotides but not the others. Results of PCR-RFLP may be achieved by visualization of fragments on a gel following restriction endonuclease digestion of the PCR product. Thus, a fragment of DNA containing the SNP is first amplified using two oligonucleotides (primers) and is subject to digestion by the variant allele-specific restriction endonuclease enzyme. If the fragment contains the variant allele it is cut into two or more pieces and in the absence of the variant allele, the PCR product remains intact. By visualizing the end-products of the digestion process by agarose or polyacrylamide gel electrophoresis, the presence or absence of the variant allele is easily detected. Other suitable methods known in the art can be used in the invention to detect the presence of SNP.

The association of a particular SNP or SNP haplotypes with disease phenotypes, such as childhood leukemia, enables the SNPs of the present invention to be used to develop superior diagnostic tests capable of identifying individuals (i.e., male and female child) who would develop childhood leukemia, as the result of a specific genotype, or individuals whose genotype places them at an increased or decreased risk of developing a detectable trait at a subsequent time as compared to individuals who do not have that genotype.

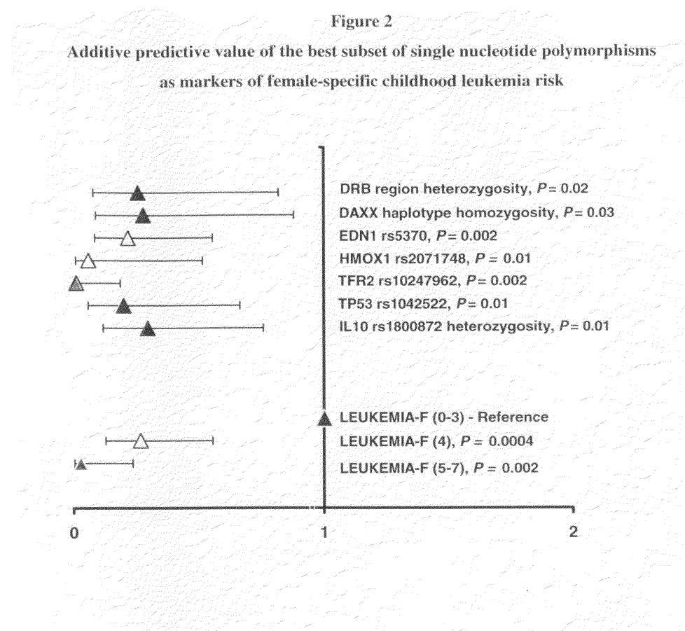

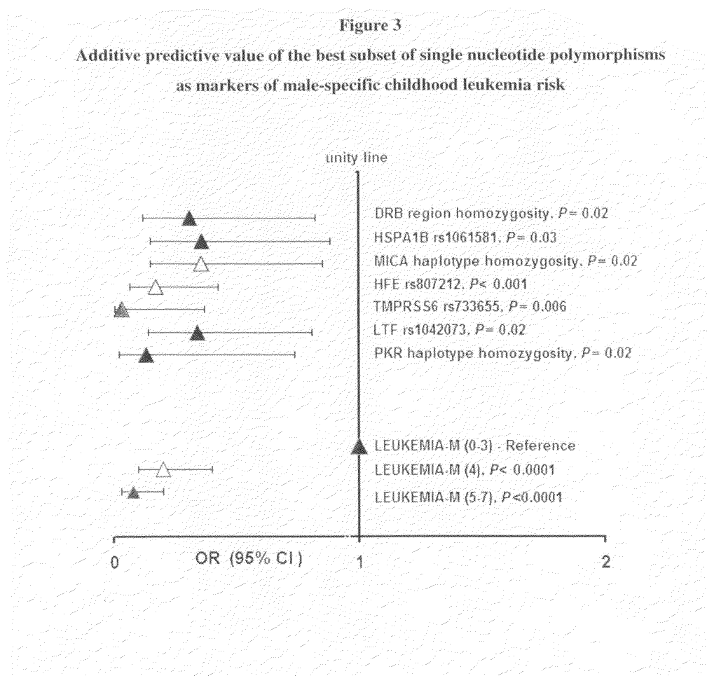

As described herein, diagnostics may be based on a single SNP or a group of SNPs. Combined detection of a plurality of SNPs (for example, 4-7) of the SNPs provided in FIGS. 2 and 3 typically increases the probability of an accurate diagnosis of predisposition. For example, in female, possession of any 4 of these markers increases the leukemia risk and having 5 or more of the markers further increases the leukemia risk with somewhat narrow confidence intervals and P values (See, FIG. 2).

In male, possession of any 4 of these markers increases the leukemia risk and having 5 or more of the markers further increases the leukemia risk with narrow confidence intervals and P values (See, FIG. 3).

The diagnostic techniques of the present invention may employ a variety of methodologies to determine whether a test subject has a SNP or a SNP pattern associated with an increased or decreased risk of developing a detectable trait as a result of a particular polymorphism, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders related to childhood leukemia.

Another aspect of the present invention relates to a method of determining whether an individual is at an increased risk (OR>1.5) or at a decreased risk (OR<0.7) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide is present at one or more SNP positions, wherein the assayed nucleotide is indicative of an increased or a decreased risk of developing the trait or indicative that the individual expresses the trait as a result of possessing a particular trait-causing or trait-influencing allele.

In one embodiment, the present invention provides a panel of individual SNPs that are useful in predicting a female-specific childhood leukemia risk. The panel of SNPs is present in several specific gene candidates including a HLA gene, iron regulatory gene, cytokine gene and other cancer-related genes (e.g., EGF, EDN1, VEGFA, TP53 and the like).

In another embodiment, the panel of SNPs in female includes at least one SNP selected from the group consisting of BMP6 rs17557, UBD rs2534790, HLA-G rs1736939, HLA-G rs1704, ZNRD1 rs9261269, DDR1 rs1264328, DDR1 rs1264323, DDR1 rs1049623, HLA-C rs9264942, SKIV2L rs419788, HLA-DRA rs3135388, DAXX rs2073524, DAXX rs1059231, and DAXX rs2239839 that is present in the HLA complex gene.

In another embodiment, the panel of SNPs in female includes at least one SNP selected from the group consisting of STEAP3 rs865688, SLC40A1 rs1439812, SLC40A1 rs1439812, HFE rs807212, TFR2 rs10247962, LCN2 rs878400, SLC11A2 rs224589, HMOX1 rs2071748, and HMOX1 rs5755709 that is present in the iron regulatory gene.

In another embodiment, the panel of SNPs in female includes at least one SNP selected from the group consisting of IL6 rs1800797 and IL10 rs1800872 that is present in the cytokine gene.

The presence of UBD rs2534790, SKIV2L rs419788, HLA-DRA rs3135388, DAXX rs2073524, DAXX rs1059231, DAXX rs2239839, SLC40A1 rs1439812, TFR2 rs10247962, or IL6 rs1800797 is indicative for an increased risk for childhood leukemia in female.

The presence of BMP6 rs17557, HLA-G rs1736939, HLA-G rs1704, ZNRD1 rs9261269, DDR1 rs1264328, DDR1 rs1264323, DDR1 rs1049623, HLA-C rs9264942, STEAP3 rs865688, HFE rs807212, LCN2 rs878400, SLC11A2 rs224589, HMOX1 rs2071748, HMOX1 rs5755709, IL10 rs1800872, or SLC40A1 rs1439812 is indicative for a decreased risk for childhood leukemia in female.

In a preferred embodiment, the presence of a combination of HLA-G rs1736939 and HLA-G rs1704 in HLA gene is indicative of a decreased risk for childhood leukemia.

In a preferred embodiment, the presence of a combination of DDR1 rs1264328, DDR1 rs1264323, and DDR1 rs1049623 in HLA gene is indicative of a decreased risk for childhood leukemia.

In a preferred embodiment, the presence of a combination of DAXX rs2073524, DAXX rs1059231, and DAXX rs2239839 in HLA gene is indicative of an increased risk for childhood leukemia.

In yet another embodiment, the present invention further provides an additional panel of individual SNPs useful in predicting female-specific childhood leukemia risk. This additional panel includes at least one SNP selected from the group consisting of EGF rs444-4903, EDN1 rs5370, VEGFA rs1570360, and TP53 rs1042522. The presence of EGF rs444-4903 or EDN1 rs5370 is indicative of a decreased risk for childhood leukemia. The presence of VEGFA rs1570360 or TP53 rs1042522 is indicative of an increased risk for childhood leukemia.

In yet another embodiment, the present invention provides a combination of at least 4 SNPs selected from the group consisting of DRB, DAXX haplotype, EDN1 rs5370, HMOX1 rs2071748, TFR2 rs10247962, TP53 rs1042522, and IL10 rs1800872, wherein the presence of said combination of the 4 SNPs is indicative of an increased risk for childhood leukemia.

In another embodiment, the present invention provides a combination of at least 5 SNPs selected from the group consisting of DRB, DAXX haplotype, EDN1 rs5370, HMOX1 rs2071748, TFR2 rs10247962, TP53 rs1042522, and IL10 rs1800872, wherein the presence of said combination of the 5 SNPs is indicative of an increased risk for childhood leukemia.

In one embodiment, the present invention provides a panel of individual SNPs that are useful in predicting a male-specific childhood leukemia risk. This panel of SNPs includes at least one SNP selected from the group consisting of NFKB1 rs4648022, MICA rs1051792, MICA STR allele 185 bp (A5.1), BAT3 rs2077102, HSPA1B rs1061581, BTNL2 rs9268480, HLA-DRA rs7192, HLA-DQA1 rs1142316, NOTCH4 rs3096702, HLA-DRB1-DQA1 rs2395225, and HLA-DRB1-DQA1 rs9271586 that is present in the HLA gene.

In another embodiment, the present invention provides a panel of SNPs that includes at least one SNP selected from the group consisting of TF rs1049296, TF rs8649, TF rs1130459, TF rs4481157, LTF rs1042073, HFE rs807212, SLC39A14 rs11136002, SLC39A4 rs2272662, LCN2 rs878400, TMPRSS6 rs733655, and TMPRSS6 rs855791 that is present in the iron regulatory gene.

In another embodiment, the present invention provides a panel of SNPs that includes at least one SNP selected from the group consisting of IL10 rs1800872, PKR rs2270414, PKR rs12712526, PKR rs2254958, CTLA4 rs231775, IRF4 rs12203592, NKG2D rs1049174, NKG2D rs2617160, NKG2D rs2734565, NKG2D rs2617170, NKG2D rs2617171, NKG2D rs1841958, NKG2D rs1983526, and IFNG rs2069727 that is present in the cytokine gene

The presence of MICA rs1051792, MICA STR allele 185 bp (A5:1), HSPA1B rs1061581, BTNL2 rs9268480, HLA-DRA rs7192, HLA-DQA1 rs1142316, NOTCH4 rs3096702, HLA-DRB1-DQA1 rs2395225, and HLA-DRB1-DQA1 rs9271586, SLC39A4 rs2272662, TMPRSS6 rs733655, CTLA4 rs231775, IRF4 rs12203592, NKG2D rs1049174, NKG2D rs2617160, NKG2D rs2734565, NKG2D rs2617170, NKG2D rs2617171, NKG2D rs1841958, or NKG2D rs1983526 is/are indicative for an increased risk for childhood leukemia in male children.

The presence of NFKB1 rs4648022, BAT3 rs2077102, HSPA1B rs1061581, TF rs1049296, TF rs8649, TF rs1130459, TF rs4481157, LTF rs1042073, HFE rs807212, SLC39A14 rs11136002, TMPRSS6 rs855791, IL10 rs1800872, PKR rs2270414, PKR rs12712526, PKR rs2254958, IFNG rs2069727, or LCN2 rs878400 is indicative for a decreased risk for childhood leukemia in male children.

In a preferred embodiment, the presence of a combination of MICA rs 1051792 and MICA STR allele 185 bp (A5.1) in HLA gene is indicative of an increased risk for childhood leukemia.

In a preferred embodiment, the presence of a combination of HSPA1B rs1061581, BTNL2 rs9268480, and HLA-DRA rs7192 in HLA gene is indicative of an increased risk for childhood leukemia.

In a preferred embodiment, the presence of a combination of HSPA1B rs1061581, HLA-DRA rs7192, and HLA-DQA1 rs1142316 in HLA gene is indicative of an increased risk for childhood leukemia.

In a preferred embodiment, the presence of a combination of HLA-DRB1-BQA1 rs2395225 and HLA-DRB1-DQA1 rs9271586 is indicative of an increased risk for childhood leukemia.

In a preferred embodiment, the presence of a combination of TF rs1049296, TF rs8649, TF rs1130459, and TF rs4481157 in iron regulatory gene is indicative of a decreased risk for childhood leukemia.

In a preferred embodiment, the presence of a combination of PKR rs2270414, PKR rs12712526, and PKR rs2254958 in iron regulatory gene is indicative of a decreased risk for childhood leukemia.

In a preferred embodiment, the presence of a combination of NKG2D rs1049174, NKG2D rs2617160, NKG2D rs2734565, NKG2D rs2617170, NKG2D rs2617171, NKG2D rs1841958, and NKG2D rs1983526 is indicative of an increased risk for childhood leukemia.

In another embodiment, the present invention provides an additional panel of SNPs in male that includes a SNP selected from the group consisting of ACP1 rs12714402, and TP53 rs1042522. The presence of ACP1 rs12714402 or TP53 rs1042522 is indicative of an increased risk for childhood leukemia.

In yet another embodiment, the present invention provides a combination of at least 4 SNPs selected from the group consisting of DRB1 region, HSPA1B rs1061581, MICA haplotype, FIFE rs807212, TMPRSS6 rs733655, LTF rs1042073, and PKR haplotype. The presence of the combination of at least 4 SNPs is indicative of an increased risk for childhood leukemia.

In another embodiment, the present invention provides a combination of at least 5 SNPs selected from the group consisting of DRB1 region, HSPA1B rs1061581, MICA haplotype, HFE rs807212, TMPRSS6 rs733655, LTF rs1042073, and PKR haplotype. The presence of the combination of at least 5 SNPs is indicative of an increased risk for childhood leukemia.

In another embodiment, the present invention provides a method of utilizing multiple SNPs that would exert joint effects and alter the individual's susceptibility to sex-specific childhood leukemia risk.

In one embodiment, the present invention provides a method of using haplotype tagging SNPs (i.e., htSNPs). htSNPs represent a cluster of SNPs in their vicinity; together, they provide additional information about genetic variation. The present invention provides a method of using the htSNP approach. When there is no already known functional SNP available in a candidate gene, the present invention provides a method of using htSNPs to predict individual's susceptibility to sex-specific childhood leukemia risk. The goal is to use functional SNPs that are known to affect either the function or expression of a gene. The use of functional SNPs may yield a positive association. On the other hand, a non-functional SNP may also be a marker to predict the risk.

Haplotype tagging SNPs are capable of representing other SNPs. This is because of a phenomenon called linkage disequilibrium (LD). Any SNP that is linked to another one via LD can be used as a substitute for the described marker. An htSNP and other SNPs tagged or represented by the htSNP form a group that are equally informative when genotyped individually. Any pair of SNPs that are in linkage disequilibrium may provide the same information. If one SNP is associated with a disease condition, the other SNP is similarly associated with the same disease condition. This generates a situation in genetic association studies where an association may be replicated by using a different SNP that is in the linkage disequilibrium with the original SNP. Accordingly, the SNPs in the present panel may be replaced by other SNPs to yield the same information. The linkage disequilibrium information is available in public resources such as HapMap (http://www.hapmap.org) or genome variation server (GVS: http://gvs.gs.washington.edu/GVS).

In one embodiment, the present invention provides a panel of SNPs, when in combination, produces a synergistic effect on sex-specific childhood leukemia risk. While an individual SNP alone may not have an effect, the combined SNPs together may exert a significant effect. In an exemplary embodiment, the presence of a combination of SNPs of HLA-DQA1 rs1142316, HLA-DRA rs7192, and HSPA1B rs1061581 is indicative of a childhood leukemia risk in males but not in females. In another exemplary embodiment, the presence of a combination of heterozygosity at HLA-G SNPs rs1736939 and rs1704 is indicative of a childhood leukemia risk in females but not in males.

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. Kits for SNP detection reagents include such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, etc.). Accordingly, the present invention further provides SNP detection kits, including but not limited to, packaged probe and primer sets (e.g., TaqMan probe/primer sets), beads that contain one or more probes, primers, or other detection reagents for detecting one or more SNPs of the present invention.

In some embodiments, a SNP detection kit typically contains one or more detection reagents and other components (e.g., a buffer, enzymes, positive control sequences, negative control sequences, and the like) necessary to carry out an assay, such as amplification and/or detection of a SNP-containing nucleic acid molecule. SNP detection kits may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention.

As will be apparent to one of skill in the art, one utility of the present invention relates to the field of genomic risk profiling. There are only a few established environmental risk markers for childhood leukemia (such as radiation exposure) with low exposure frequencies (Linet et al, 2003). Thus, genomic risk profiling is superior to environmental risk profiling for childhood leukemia. After the genotyping assessment of the presence of specific SNPs in a child, a physician can thereby predict the sex-specific childhood leukemia development probability.

### EXPERIMENTAL STUDIES

### Example 1

### Characteristics of Patient and Control Samples

We used patient and control sample set to seek childhood leukemia associations in the various genes (e.g., HLA complex). This sample set contains 114 cases with childhood ALL (<15 year-old) and 388 newborn controls from South Wales, U.K. The childhood ALL cases were consecutively diagnosed from 1988 to 1999 in South Wales (U.K.). The use of a newborn control group allows estimation of the leukemia risk for a newborn.

The control sample set consists of 388 cord blood samples from 201 girls and 187 boys. The cord blood samples from newborns or peripheral blood samples from leukemia cases were collected in EDTA-containing tubes. White blood cells were isolated using standard protocols. DNA was extracted from white blood cells using standard phenol-chloroform extraction method or equivalent methods. DNA samples were re-suspended in double distilled H₂O at 100 nanograms per microliter (ng/mL) and kept frozen at −20° C. until used for genotyping. Further details of the samples are provided in detailed experimental procedures section.

Table 1 lists all of the 311 SNPs from the candidate genes we selected to test for their predictive value for childhood leukemia risk. The table provides the gene name, the SNP ID number (beginning with rs) as listed in National Center for Biotechnology Information (NCBI) Entrez SNP (http://www.ncbi.nlm.nih.gov/sites/entrez?db=snp) (the disclosure of which is incorporated herein by reference), chromosomal location and the position in the chromosome as nucleotide number beginning from the tip of the short arm of a chromosome.

Each one of the 311 SNPs from our candidate genes were genotyped in newborns and genotype frequencies were compared between cases and controls for each sex. Any difference between the frequencies was considered to be an indication of the involvement of the SNP in risk.

### Example 2

### Selection of Genes for Testing their Role in Childhood Leukemia Development

To the best of the present inventors' knowledge, despite few published reports including those by the inventor cited elsewhere in this application and by others (See, for example, Shannon K, 1998; Canalle et al, 2004; Sinnett et al, 2006; Chokkalingam & Buffler, 2008), there are no genetic polymorphisms for prediction of childhood leukemia risk in clinical use. This is partly due to very low predictive value provided by individual markers. When combined, however, the cumulative or additive predictive may sum up to remarkable values. The present application demonstrates the feasibility of this approach that has not been tried in childhood leukemia before.

The present inventors used recently emerged information on the genomic polymorphisms in genes likely involved in childhood leukemia development. We recognized the sex-specific differences in risk to develop childhood leukemia. Because males and females may be influenced in opposite directions by the same gene polymorphisms, unless stratified by sex, an overall analysis may obscure the predictive value of a marker.

While any gene may have a role in childhood leukemia development, we stratified the genes for the probability of their involvement and used a candidate gene approach. Besides known physiologic roles of genes, we also exploited our own findings in prenatal selection since susceptibility to leukemia and prenatal selection share genetic risk markers (Dorak et al, 2007). Furthermore, childhood leukemia is more common in males and since we explored markers for sex-specific leukemia risk, we included markers for male-specific prenatal selection.

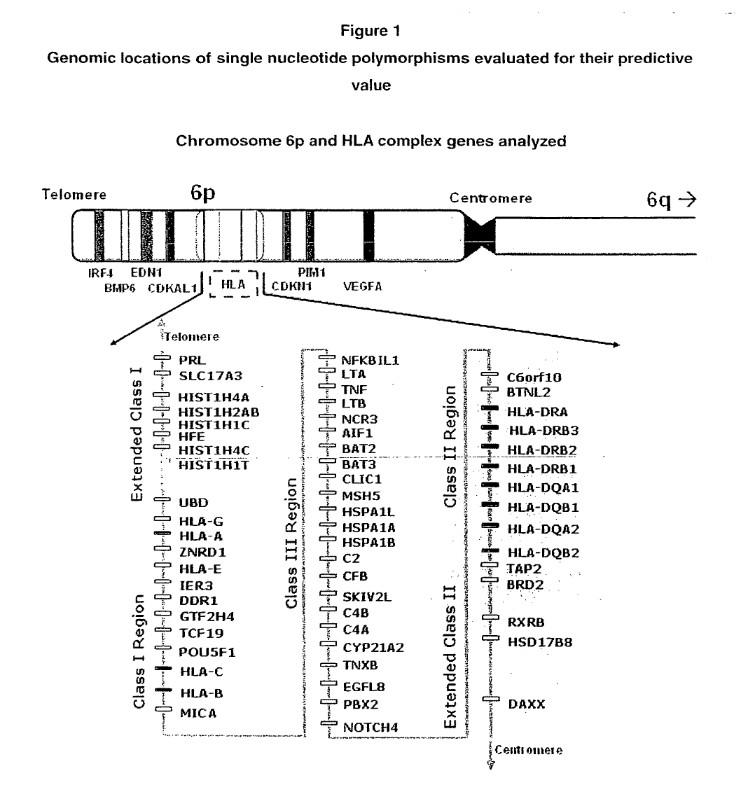

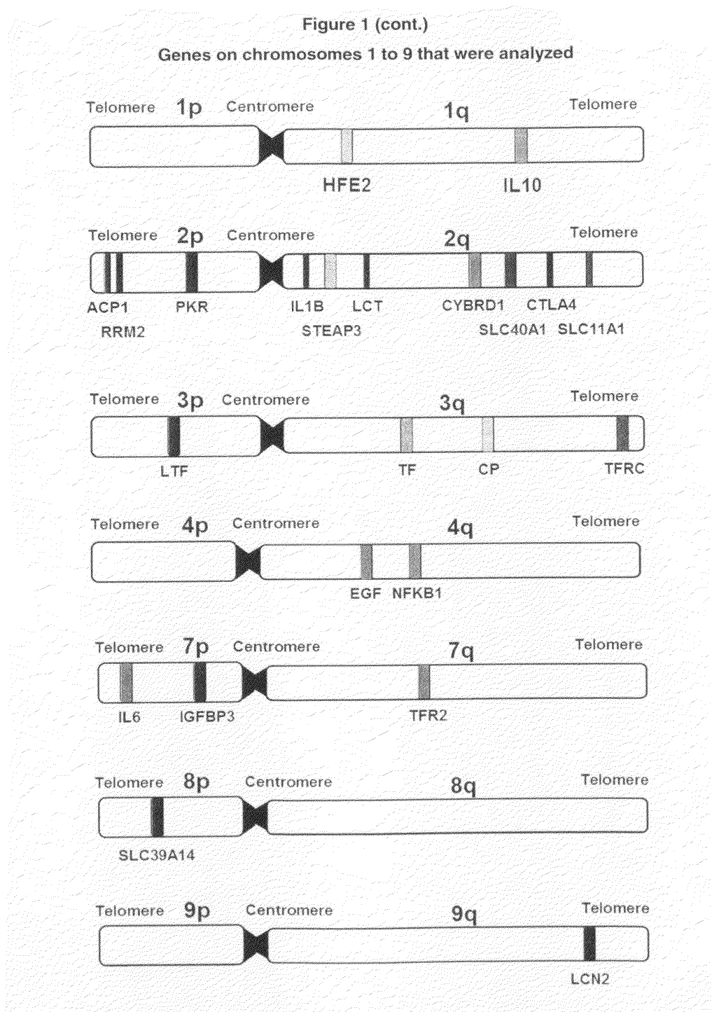

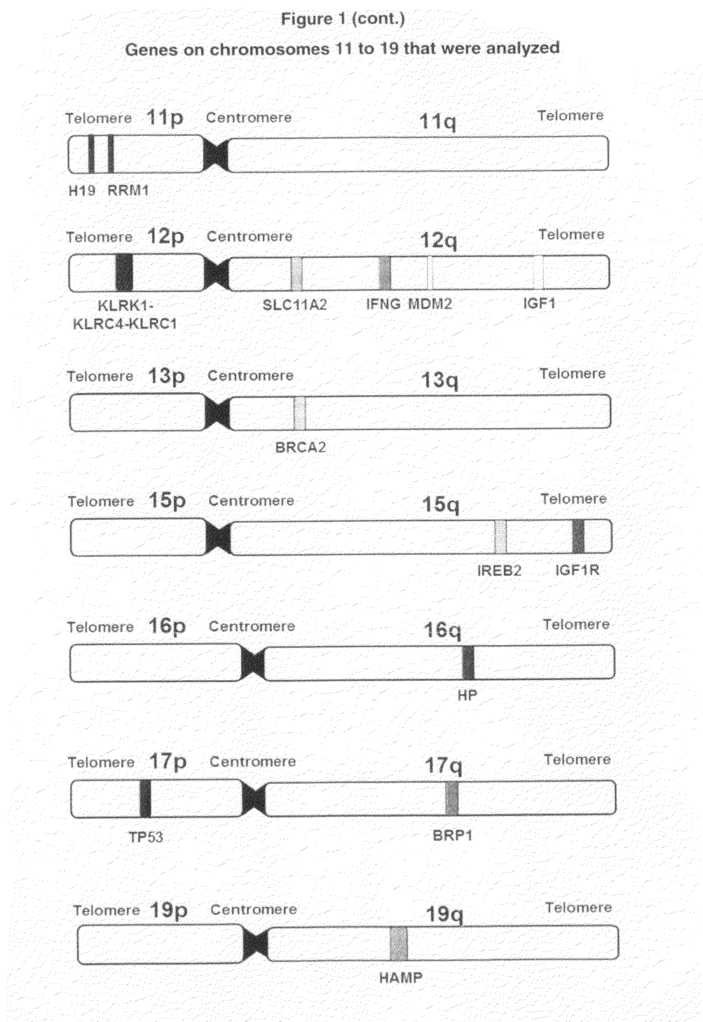

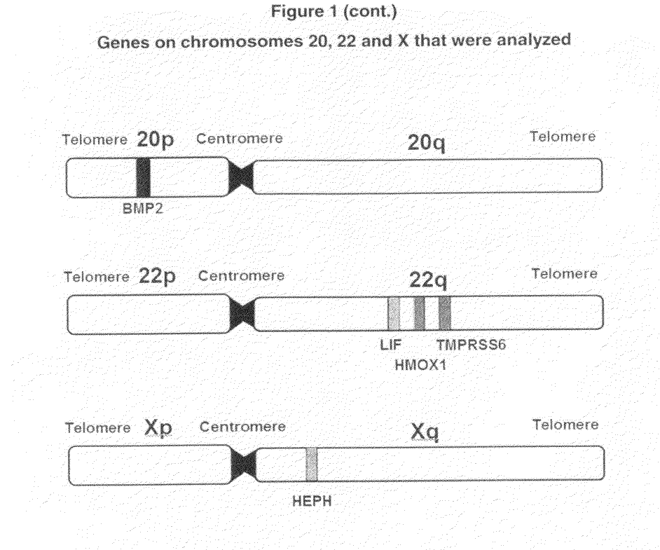

We found that most of these markers are from the HLA complex and iron regulatory genes, as well as selected cytokine genes IFNG, IL10 and IL6 (See, Table 1 and FIG. 1). These three groups of genes, e.g., HLA complex, iron-regulatory and immune surveillance-related genes, represent plausible gene candidates for childhood leukemia development.

We chose to examine additional cancer-related gene candidates. These include vascular endothelial growth factor type A (VEGFA), endothelin-1 (EDN1), leukemia inhibitory factor (LIF), tumor protein p53 (TP53), its regulator MDM2, natural killer cell receptor (NKG2D also known as KLRK1) and acid phosphatase type 1 (ACP1) due to their individual merits. We analyzed selected polymorphisms of these relevant genes in the potential genetic marker list (See, Table 1, and FIG. 1).

### Example 3

### Genotypings of Single Nucleotide Polymorphisms

We achieved genotyping of SNPs using a variety of methods. We found that they consistently provide equivalent results. The choice was based on availability of the necessary instruments and expertise, budget available for the study and convenience. Our choice of method was TaqMan allelic discrimination assay for SNP genotyping. All TaqMan assays were purchased from ABI (ABI, Foster City, Calif.).

When TaqMan allelic discrimination assay was not possible to use, we chose an alternative method. This happened for HLA-DRA rs3135388, HLA-DQA1 rs1142316, HLA-G rs1704, HSPA1B rs1061581, MICA rs1051792 and HMOX1 rs5755709. For these polymorphisms, we used a PCR based restriction fragment length polymorphism assay. The details of these methods used to genotype polymorphisms within our candidate genes are provided in the detailed experimental procedures section.

Table 2 shows the 73 SNPs either showed an individual difference in genotype frequencies between male and female cases and controls or contributed to a combination of regional genotype combinations that showed frequency differences or that gained statistical significance in the multivariable model. The gene name, SNP ID number, alternative name for the SNP according to Genome Variation Society (HGV), when available, SNP location within the gene and nucleotide change are shown.

### Example 4

### Heterozygote Advantage in Childhood Leukemia Risk Prediction

In this series of study, we examined heterozygosity at all SNPs for its effect on sex-specific childhood leukemia susceptibility. Heterozygosity rates were calculated as the number of samples coded as 1 divided by the total number of samples (those coded as 0 plus 1). This calculation was done separately for case and control groups and also for males and females separately in each group. The comparisons between cases and controls for the overall groups, boys and girls were done by using logistic regression analysis (equivalent to 2×2 contingency table analysis by Chi-squared or Fisher's exact test) to obtain an odds ratio (OR, fold change in risk to develop leukemia), its 95% confidence interval (95% CI) and a two-tailed P value.

The results suggested that also in childhood leukemia genome-wide heterozygosity is protective for childhood leukemia development. The SNPs at HFE, EDN1, BMP6, SLC39A14, SLC40A1, TF, LCN2, EGF, IL10, IFNG and NFKB1 showed reduced frequencies in cases compared with newborns (See, Tables 3 & 5). Out of these, only the IL10 SNP rs1800872 remained statistically significant to be represented in the final female-specific predictive model (Table 4, FIG. 2).

In this DNA-level systematic study of HLA complex heterozygosity in any disease, multiple SNPs at the HLA complex genes including UBD, ZNRD1, IER3, DDR1, TCF19, POU5F1, MICA, NCR3, BAT3, CLIC1, MSH5, HSPA1L/A/B, SKIV2L, CYP21A2, PBX2, NOTCH4, C6orf10, BTNL2, BRD2, RXRB and DAXX as well as at SNPs at the HLA genes HLA-C, -DRA, -DQA1 and DRB1-DQA1 region were genotyped by TaqMan allelic discrimination assays, high-resolution melting analysis with unlabeled probes or PCR-restriction fragment length polymorphism (RFLP) analysis in childhood leukemia cases and newborn controls. At each SNP, heterozygotes were coded as “1” and homozygotes were coded as “0” for subsequent statistical analysis.

The SNPs at BAT3 (rs2077102), ZNRD1 (rs9261269), multiple SNPs at DDR1 (rs1264328-rs1264323-rs1049623), HLA-G (rs1736939, rs1704) and DRB1-DQA1 region (rs2395225, rs9271586) in combinations showed reduced heterozygosity frequencies in cases compared with newborns (See, Tables 3 & 5). HLA-DR region SNPs rs2395225 and rs9271586 in combination was the only marker retained in the final predictive model for female-specific leukemia risk (Table 4, FIG. 2).

### Example 5

### Genetic Markers from Non-HLA Genes of the HLA-Complex that Predict Childhood Leukemia Risk

We identified genetic markers that represent main lineages of HLA haplotypes. The first set of these genetic markers are: (i) HSPA1B rs1061581; (ii) HLA-DRA rs7192; and (iii) HLA-DQA1 rs1142316. The major alleles of these SNPs characterize the ancestral HLA-DRB4 lineage (i.e., HLA-DR4, HLA-DR7 and HLA-DR9). The minor alleles of these SNPs characterize the HLA-DRB3 lineage (i.e., HLA-DR3, HLA-DR11/12 and HLA-DR13/14). Likewise a similar set of three SNPs (HSPA1B rs1061581, HLA-DRA rs7192, and BTNL2_rs9268480) also showed a similar association in males (Table 5). None of these SNPs are from the coding regions of classical HLA genes that have shown inconsistent associations with leukemia susceptibility in earlier studies (Bortin et al, 1987). The SNPs in the second set are from the HLA-DRB1-DQA1 region (See, FIG. 1) and again not from coding regions of HLA genes. These are: (i) rs2395225 and (ii) rs9271586.

These two sets of SNPs showed sex-specific associations with childhood leukemia risk. As mentioned above, in females, although only marginally significant in univariable analysis (OR=0.41, 95% CI=0.17 to 1.02; P=0.06) and not listed in Table 3, heterozygosity for both DRB1-DQA1 region SNPs (rs2395225 and rs9271586) in combination reached statistical significance as an independent protective marker in the final multivariable model (FIG. 2). In males, homozygosity the same two SNPs (rs2395225 and rs9271586) increased the risk (Table 5) and this combined marker was retained in the final model (Table 6, FIG. 3). In males, the other sets of SNPs also showed risk associations but their association was not independent and was represented by the DRB1-DQA1 region SNPs in the final model.

Besides those that showed protective associations in the form of heterozygote advantage and mentioned above, other non-HLA genes of the HLA complex that showed associations included the HLA-DRA and HLA-C associations in females, and NOTCH4, HSPA1B, BAT3 SNPs and a combined MICA genotype in males. Of these, the HSPA1B rs1061581 and combined MICA genotype consisting of the SNP rs1051792 and exon 5 STR were strong and independent enough to remain in the final male-specific predictive model (Table 6, FIG. 3). In females, the only HLA complex marker in the final model (other than the DRB1 region heterozygosity) was DAXX haplotype homozygosity. This non-HLA gene haplotype consists of three SNPs (rs2073524, rs1059231, rs2239839). Its association was female-specific and remained in the final predictive model (Table 4, FIG. 2).

### Example 6

### Genetic Markers from Outside the HLA-Complex that Predict Childhood Leukemia Risk: Iron-Related Gene Polymorphisms

Iron is a required element for cellular proliferation and a nutrient for cancer cells. We examined the plausibility that iron regulatory gene polymorphisms may influence body iron levels and thereby modify childhood cancer susceptibility as well as other cancers (Dorak et al, 2005). The first iron-related gene polymorphism association was between HFE gene variant C282Y and childhood leukemia and was shown by the inventor (Dorak et al, 1999b). In the present application not only the HFE gene was examined in greater detail but other iron regulatory gene polymorphisms were also investigated.

In females, HFE region SNP rs807212 heterozygosity showed a protective association but more importantly a number of iron regulatory gene SNPs showed associations in univariable analysis. These included BMP6, LCN2, HMOX1, TFR2, STEAP3, SLC11A2 and SLC40A1 (Table 3). Of these the HMOX1 rs2071748 and TFR2 rs10247962 associations were strong enough and independent to remain in the final predictive model (Table 4, FIG. 2).

In males, several iron regulatory gene SNPs showed protective associations in heterozygous form (HFE, TF, LCN2, SLC39A14) (Table 5). However, other iron regulatory genes such as TMPRSS6, TF, LTF and SLC39A4 showed some of the strongest associations (Table 5). Of these HFE rs807212, TMPRSS6 rs733655 and LTF rs1042073 associations remained in the final predictive model as independent markers of male-specific childhood leukemia susceptibility (Table 6, FIG. 3).

### Example 7

### Genetic Markers from Outside the HLA-Complex that Predict Childhood Leukemia Risk: Cytokine and Other Immune Surveillance-Related Genes

We examined an IFNG SNP (rs2069727) because of its sex-specific expression patterns. This SNP showed different genotype frequencies between male cases and controls (Table 5). Among other cytokine gene polymorphisms, IRF4 rs12203592 homozygosity, IL10 rs 1800872 heterozygosity and NFKB1 rs4648022 heterozygosity were associated with male-specific childhood leukemia susceptibility. The PKR gene (formally known as EIF2AK2) also encodes a product that is involved in immune response (interferon-inducible elF2alpha kinase). Analysis of three SNPs in PKR (rs2270414, rs12712526, rs2254958) only showed a marginally significant association with combined wildtype homozygosity at all three SNPs in males (OR=0.45, 95% CI=0.20 to 1.02; P=0.06). However, this combined genotype appeared as a stronger, independent marker of susceptibility in the male-specific predictive model (Table 6, FIG. 3).

In females, IL6 promoter region SNP rs1800797, selected because of its association with hyperandrogenism, showed a strong association in univariable association (Table 3). The only immune regulatory gene polymorphism (from outside the HLA complex) that was represented in the final female-specific model was IL10 rs1800872 heterozygosity (Table 4, FIG. 2).

In the NKG2D (KLRK1) gene and in its flanking region, a seven SNP haplotype consisted of rs1049174-rs2617160-rs2734565-rs2617170-rs2617171-rs1841958-rs1983526 conferred risk for childhood leukemia in homozygous form without sex specificity (OR=2.58, 95% CI=1.25 to 5.30; P=0.01). The association was still strong in each sex (OR=2.46 in males and 2.60 in females) but with only marginal statistical significance because of low frequency of this genotype. However, in combination with HSPA1B SNP rs1061581 variant allele positivity, the same NKG2D showed an even stronger association again with no sex specificity (OR=4.05, 95% CI=1.60 to 10.3; P=0.004).

CTLA4 SNP rs231775 was examined as an important immune system-related gene marker. Homozygosity for the variant allele of this SNP was associated with increased risk for childhood leukemia in males only (OR=2.28, 95% CI=1.06 to 4.68, P=0.04).

### Example 8

### Other Genetic Markers that Predict Childhood Leukemia Risk

Certain genetic polymorphisms were included in the analysis because of their individual merits. Of those, ACP1 SNP rs1274402 variant homozygosity showed a strong risk association in males (OR=2.48, 95% CI=1.09 to 5.65; P=0.03). In females, VEGFA rs1570360 variant homozygosity (OR=2.47, 95% CI=1.03 to 5.89; P=0.04) and EDN1 rs5370 variant allele positivity (OR=0.36, 95% CI=0.17 to 0.77; P=0.008) showed strong associations.

A TP53 coding region SNP (rs1042522, R72P) was examined because of its associations with other cancers. It was a strong marker for risk overall which reached statistical significance in females (OR=3.50, 95% CI=1.40 to 8.76; P=0.008) and remained in the final predictive model (Table 4, FIG. 2).

### Example 9

### Multivariable SNP Analysis and Generation of Final Predictive Models for Each Sex

The risk for childhood leukemia is not determined by a single genotype and our single marker analysis revealed multiple statistically significant associations. We therefore proceeded to the next step and analyzed the statistically significant or marginally significant associations by multivariable modeling to identify the most informative minimal subset of markers. These would be the statistically most significant and independent associations. Independence is important to avoid redundancy in testing samples and also for contributions to the additive model. Markers that are correlated and therefore not independent do not add to the information obtained from one of them and does not change the odds ratio when included in the multivariable final model.

The multivariable modeling yielded the independence and statistical significance of the markers included in the top portions of FIGS. 2 (females) and 3 (males). In these final models, all adjusted odds ratios were smaller than 0.50 and therefore associated with more than twice increased risk for childhood leukemia. (See FIGS. 2 & 3 legends for detailed explanation). Each model consisted of seven independent markers of susceptibility (Tables 4 (females) and 6 (males)).

Next, we assessed the value of this subset of markers in predicting the risk jointly. After arranging all associations to be in the same direction, it was possible to examine the additive effect of the sum of markers without any further manipulation. Each individual was simply given a score for the number of markers possessed. Thus, the scores were between 0 and 7. Each group was stratified into three groups: the baseline group consisted of subjects possessing any 0 to 3 of the seven markers, the next group consisted of subjects who possessed any 4 of the seven markers and the third group was positive for any 5 or more of the seven markers.

Examination of the additive effect of seven sex-specific markers of susceptibility revealed a stepwise progression in odds ratio corresponding increasing risk as the number of markers possessed increases. The overall model reached extreme statistical significance for each sex (P<10⁻⁶). These figures translate into more than ten times increased risk for newborns possessing five or more of the seven markers.

### EXPERIMENTAL PROTOCOLS

### I. Characterization of Clinical Samples

The population sample analyzed in this study consisted of anonymously collected cord blood samples from newborns and peripheral blood samples from childhood leukemia (ALL) cases in South Wales (United Kingdom). Random, anonymous umbilical cord blood samples were obtained from full-term babies born in the University Hospital of Wales and Llandough Hospital in Cardiff, UK over a period of 12 months from 1996 (Dorak et al, 2002b). Leukemia cases represent all but four cases diagnosed over a ten-year period in South Wales (Dorak et al, 1999a). This practice of collection of surplus biological material for research purposes anonymously was in compliance with the regulations of the local institutional ethics committee.

It was not practically possible to obtain samples from every newborn over this period but no newborn was intentionally excluded on the basis of any selection criteria. The samples were collected until the number in both sex groups exceeded 200. In the final group of 415 newborns, there were 201 boys and 214 girls. This gives a male-to-female (M:F) ratio of 0.939 that is slightly lower than the expected M:F ratio (1.056) in newborns (statistically non-significant).

In the present study, 388 of the originally collected 415 samples were genotyped due to limited DNA availability (201 girls and 187 boys). No data are available about the newborns (such as gestational age, birth order, birth weight, parental age) other than their sex and that they were born via natural vaginal birth. No newborn born via cesarean section was included.

The preference of newborns as a control group has a scientific basis. A previously published study reported a strong risk association with homozygosity for HLA-DRB4 (having two copies of HLA-DRB4 gene) and this association was observed in boys only (Dorak et al, 1999a). That study was a strong indicator that HLA influence on leukemia development was sex-specific. The newborn control group was also studied separately (Dorak et al. 2002b) to examine whether newborn boys and girls had different genotype frequencies as a result of different selective pressure during prenatal period. This was indeed the case and combined homozygosity for HLA-DRB4 and -DRB3 genes was decreased in boys. A hypothesis was advanced that homozygosity at the HLA complex was deleterious for boys during prenatal development and boys with homozygous HLA genotypes are lost preferentially. Those who survive prenatal selection are at higher risk to develop childhood leukemia. This hypothesis is best tested using a newborn control group and this would also allow estimation of the leukemia risk for a newborn.

### II. Genotyping Procedures

(A) Allelic discrimination assays

Allelic discrimination assays were performed on Stratagene MX3000P instruments. The standard thermal profile protocol was used with the modification of 90 sec at 60° C. for 50 cycles. TaqMan® SNP genotyping assay purchased from ABI as 40× was diluted to 20× by adding Tris-HCl and EDTA at pH 8.0. 96-well plates were set up by adding 1.5 μl DNA (10 μg/l), 4.625 μl ddH₂O and 6.25 μl TaqMan® genotyping master mix (ABI) and 0.625 μl assay reagents. Each plate contained intra and inter-plate controls and no-template controls. Built-in Stratagene Mx3000P software was used to assign genotypes.

(B) Polymerase Chain Reaction—Restriction Fragment Length Polymorphism (PCR-RFLP) Analysis

PCR-RFLP analysis was performed to genotype the HSPA1B SNP rs1061581 using oligonucleotides 5′-CAT CGA CTT CTA CAC GTC CA-3′ (SEQ ID NO: 1) and 5′-CAA AGT CCT TGA GTC CCA AC-3′ (SEQ ID NO: 2) and the restriction endonuclease PstI. In the first step, using the oligonucleotides, a 1,117 bp fragment was amplified with 15 ng genomic DNA by the following conditions; 10×PCR buffer, 6.25 mM 2′-deoxyribonucleotide 5′-triphosphate (dNTP) mix, 1.2 μM of each primer, 0.6 mM MgCl₂ and 1.0U Taq polymerase (Mango Taq, Bioline USA, Inc, Randolph, Mass.) in a final volume of 250.

The PCR amplification was set up with the initial denaturation at 95° C. for 5 min, 35 cycles at 95° C. for 30 sec, 58° C. for 30 sec, 72° C. for 1 min and a final extension at 72° C. for 10 min (TGradient Thermoblock, Biometra, Goettingen, Germany). The fragments were then subjected to restriction endonuclease digestion by using the PstI enzyme. This enzyme cuts the fragment into two fragments of 934 bp and 183 bp when there is a nucleotide G in the SNP position but fails to cut it when there is a nucleotide A in the SNP position. Samples with only 934 bp and 183 bp fragments were classified as homozygote for allele G and samples with only the 1,117 bp fragment were classified as homozygote for allele A. Samples that contained 1,117 bp, 934 bp and 183 bp fragments were classified as heterozygote for alleles A and G.

PCR-RFLP analysis was performed to genotype the HLA-DQA1 3′UTR SNP rs1142316 using oligonucleotides 5′-CAA GGG CCA TTG TGA ATC YCC AT-3′ (SEQ ID NO: 3) and 5′-TGG GYG GCA RTG CCA A-3′ (SEQ ID NO: 4) and the restriction endonuclease BglII. In the first step, using the oligonucleotides, a 726 bp fragment was amplified with 15 ng genomic DNA by the following conditions; 10×PCR buffer, 2.4 mM 2′-deoxyribonucleotide 5′-triphosphate (dNTP) mix, 1.2 μM of each primer, 0.6 mM MgCl₂ and 1.0U Taq polymerase (Mango Taq, Bioline USA, Inc, Randolph, Mass.) in a final volume of 250. The PCR amplification was set up with the initial denaturation at 95° C. for 5 min, 35 cycles at 95° C. for 30 sec, 57° C. for 30 sec, 72° C. for 1 min and a final extension at 72° C. for 10 min (TGradient Thermoblock, Biometra, Goettingen, Germany). The fragments were then subjected to restriction endonuclease digestion by using the BglII enzyme. This enzyme cuts the fragment into two fragments of 513 bp and 213 bp when there is a nucleotide C in the SNP position but fails to cut it when there is a nucleotide A in the SNP position. Samples with only 513 bp and 213 bp fragments were classified as homozygote for allele C and samples with only the 726 bp fragment were classified as homozygote for allele A. Samples that contained 726 bp, 513 bp and 213 bp fragments were classified as heterozygote for alleles A and C.

MICA-V152M exon 3 was PCR amplified with forward primer 5′-CGGGAATGGAGAAGTCACTGCT-3′ (SEQ ID NO: 5) and reverse primer 5′-CAACTCTAGCAGAATTGGAGGGAG-3′ (SEQ ID NO: 6) for rs1051792 SNP genotyping. The 50 μl final reaction volume consisted 30 ng genomic DNA, 5×PCR buffer, 75 mM MgCl₂, 2.4 mM 2′-deoxyribonucleotide 5′-triphosphate (dNTP) mix, 2.4 μM of each primer and 0.3U Taq polymerase (Platinum Taq, Invitrogen, Roche Molecular Systems, Inc, Alameda, USA & ABI, Foster City, Calif.).

Touchdown PCR was set up with the initial denaturation at 95° C. for 5 min, 5 cycles at 95° C. for 30 sec, 60° C. for 30 sec, 72° C. for 1 min, 10 cycles at 95° C. for 30 sec, 59° C. for 30 sec, and 20 cycles at 95° C. for 30 sec, 58° C. for 30 sec, 72° C. for 1 min followed by a final extension at 72° C. for 10 min. Digestion with HpyCH4III yielded two constant bands 211 bp and 162 bp for minor allele A and a 373 bp band for the major allele G.

(C) PCR Analysis of an Insertion/Deletion Polymorphism

The 14 bp insertion/deletion polymorphism in HLA-G (rs1704) was performed by electrophoresis. HLA-G exon 8 was amplified by PCR using the forward primer 5′-GGTCTCTGACCAGGTGCTGT-3′ (SEQ ID NO: 7) and reverse primer 5′-GGAATGCAGTTCAGCATGAG-3′ (SEQ 1D NO: 8). 15 ng genomic DNA by the following conditions; 10×PCR buffer, 1.2 mM 2′-deoxyribonucleotide 5′-triphosphate (dNTP) mix, 1.2 μM of each primer, 0.6 mM MgCl₂ and 1.0U Taq polymerase (Mango Taq, Bioline USA, Inc, Randolph, Mass.) in a final volume of 25 μl. The PCR amplification was set up with the initial denaturation at 95° C. for 5 min, 35 cycles at 95° C. for 30 sec, 62° C. for 30 sec, 72° C. for 1 min and a final extension at 72° C. for 10 min (TGradient Thermoblock, Biometra, Goettingen, Germany). The expected amplicon sizes were either 400 bp with the insertion or 386 bp with the deletion of 14 bp in exon 8. PCR products were run on 2.5% agarose gels and scored by observation.

(D) Short Tandem Repeat (STR) Polymorphism Genotyping

The MICA gene was selectively amplified by using forward primer 5′-CCTTTTTTTCAGGGAAAGTGC-3′ (SEQ ID NO: 9) (labeled with Cy at the 5′ end) and reverse primer 5′-CCTTACCATCTCCAGAAACTGC-3′ (SEQ ID NO: 10) for genotyping the STR locus in exon 5. 15 ng genomic DNA by the following conditions; 10×PCR buffer, 1.2 mM 2′-deoxyribonucleotide 5′-triphosphate (dNTP) mix, 0.4 μM of each primer, 0.6 mM MgCl₂ and 1.0U Taq polymerase (Mango Taq, Bioline USA, Inc, Randolph, Mass.) in a final volume of 250.

The PCR amplification was set up with the initial denaturation at 95° C. for 5 min, 35 cycles at 95° C. for 30 sec, 62° C. for 30 sec, 72° C. for 1 min and a final extension at 72° C. for 10 min (TGradient Thermoblock, Biometra, Goettingen, Germany). PCR products were cleaned up by using QIAGEN QIAquick PCR Purification Kit and run on Beckman Coulter CEQ™ 8000 Genetic Analysis System in the presence of molecular size markers for accurate sizing of the fragments.

(E) High Resolution Melting Analysis for Genotyping

High resolution melting analysis was performed to genotype HLA-DRA rs3135388. Idaho Technology Light Scanner primer design software was used to design the oligonucleotides 5′-TGCATTCTGAGATCCATACCTT-3′ (SEQ ID NO: 11) and 5′-TTCATCAGACATATCCCGGTTC-3′ (SEQ ID NO: 12) and the probe 5′-TCTCCCAACAAACCAATCCCACTTTAGG (SEQ ID NO: 13)/3Amm/-3′. In the first step the asymmetric PCR reaction contained a final concentration of 1×LCGreen MasterMix (Idaho Technology Inc, Salt Lake City, Utah), 0.2 mM forward primer, 1.0 mM reverse primer, 0.6 mM probe (3′ blocked), 10 ng genomic DNA and water to raise the final volume to 5 ml. We amplified the target with a final step to induce heteroduplexes: 95° for 5 minutes; then 45 cycles of 95° C. for 30 sec, 68° C. for 30 sec, and 72° C. for 30 sec and a final melt at 95° C. for 30 sec, then a rapid cooling to 20° C. The plate was then inserted into LightScanner (LightScanner, Idaho Technologies, Utah) by setting the melting temperatures between 45° C. and 95° C. High resolution melting program was run.

High resolution melting analysis was used to genotype HMOX1 rs5755709 using the Idaho Technology Light Scanner primer design software. The asymmetric PCR reaction contained a final concentration of 1×LCGreen MasterMix (Idaho Technology Inc, Salt Lake City, Utah), 0.2 mM forward primer, 1.0 mM reverse primer, 0.6 mM probe (3′ blocked), 10 ng genomic DNA and water to raise the final volume to 5 ml. We also used HRM analysis to genotype the samples with primers 5′-ACAGAGTGAGACCCCATCGCA-3′ (SEQ ID NO: 14) and 5′-TGTCTTCCTGGGGCCTCAGTTT-3′ (SEQ ID NO: 15) and the probe 5′-TAAGTGAACAAGAAATTATCTTTATTCCC-3′ (SEQ ID NO: 16). We amplified the target with a final step to induce heteroduplexes: 95° C. for 5 minutes; then 45 cycles of 95° C. for 30 sec, 68° C. for 30 sec, and 72° C. for 30 sec and a final melt at 95° C. for 30 sec then a rapid cooling to 20° C. The plate was then inserted into LightScanner (LightScanner, Idaho Technologies, Utah) and melted the PCR product from 55° C. to 75° C.

Table 7 shows the flanking DNA sequence of each SNP. The SNPs are shown as the wild-type and variant alleles. Table 8 lists the different genotyping methods used to genotype SNPs analyzed in this invention.

### III. Statistical Analysis

The statistical analysis of a SNP association may be performed using the following statistical models. It may be of importance to have the variant allele in homozygous or heterozygous combination as long as there is at least one copy of it in the genotype (CT and TT). In this case, individuals with CT or TT genotypes are pooled together and coded as 1 in a variable that are going to be used in the statistical analysis. The code 1 indicates presence of the susceptibility marker. In this case, individuals who have the homozygous wild-type genotype are coded as 0 meaning the lack of the susceptibility marker. This model that pools heterozygotes and homozygotes together is called dominant genetic model and can also be described as variant allele positivity.

In recessive model, the interest is on homozygous genotype of the variant allele (TT) and individuals with the TT genotype are coded as 1 while all other genotypes are coded as 0. This model is called recessive model and can also be described as variant allele homozygosity.

There are certain situations in which the number of variant allele possessed is important because having 1 or 2 copies of the variant allele correlates with the degree of susceptibility. In this case, individuals with genotype CT (one copy of the variant allele) have increased susceptibility and individuals with genotype TT (two copies of the variant allele) have an even higher degree of susceptibility. This model is called the additive model and demonstrates a gene-dosage effect. In most cases, statistical significance for this model is usually an indication of an association with dominant or recessive model. In our analysis that follows, we have presented dominant or recessive model associations for each SNP. Variables with P values of less than 0.05 were considered statistically significant. Statistical association analysis was carried out using logistic regression with Stata version 10 statistical software.

One exceptional situation is that the heterozygous genotype CT may be of importance. Heterozygosity in the genome is shown to be a beneficial trait for prevention from many common diseases including infections and cancer. This situation is called ‘heterozygote advantage’ and is characterized by decreased frequency or underrepresentation of a heterozygous genotype among cases with a disease compared with normal controls because of its protective effect from the condition.

As mentioned above, each individual is coded as 0 or 1 based on the absence or presence of the susceptibility genotype(s) for each SNP before statistical association analysis. A SNP may have a deleterious or beneficial effect on a condition. In the present invention, the outcome of interest was sex-specific susceptibility to childhood leukemia. In this case, risk genotypes are overrepresented and protective genotypes are underrepresented in cases in comparison to controls. To avoid elaborate mathematical manipulations while constructing a statistical model to find the most informative subset of SNPs with cumulative effects, it is desirable that all SNPs are beneficial or deleterious, i.e., all SNPs act in the same direction. This means, it is easier to construct a model if the direction of the effect is the same for each SNP. In the case of SNP associations, this is achieved easily. Since each individual is coded as 0 or 1, when necessary, an association that is deleterious can be converted to a protective one by simply reversing the statistical codes. All results presented in the final multivariable models are in the direction of protection. In terms of the odds ratio, which is a measure of the strength of association, they are all less than 1.0 in the final models (presented in FIGS. 2 and 3) and its distance from 1 (or its proximity to 0) is an indication of the strength of the association. Thus, a value of 0.49 suggests, a newborn with this genotype has a 51% increased risk for childhood leukemia compared to the newborns in the reference group.

The direction of protection was preferred over the direction of risk because of a mathematical property of the odds ratio. Protective odds ratios lie between 0 and 1 but risk odds ratios lie between 1 and infinity. An odds ratio for a protective association makes more intuitive sense than an odds ratio in the risk direction especially when two odds ratios are compared. For this reason, we chose to convert all associations to protective direction by converting the statistical coding when necessary. Thus, if a dominant model risk association was observed for a SNP, it was presented as it is in the univariable associations (Table 3 for females and Table 5 for males) but converted to a protective one by reversing the coding in the multivariable model. When this is done, a dominant risk association becomes a protective association for wildtype homozygous genotype. The conversion of a protective odds ratio to a risk odds ratio for the opposite genotype is simple. The reciprocal (1 divided by the value) of a protective odds ratio gives the risk odds ratio for the opposite genotype. Thus, a protective odds ratio of 0.50 for wildtype homozygosity corresponds to odds ratio=2.0 for the dominant model (variant allele positivity) of the same SNP.

All patents, publications, accession numbers, and patent application described supra in the present application are hereby incorporated by reference in their entirety.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### REFERENCES

1. Bortin M M, D'Amaro J, Bach F H, Rimm A A, van Rood J J. HLA associations with leukemia. Blood 1987:70(1):227-32 (full-text: http://bloodjournal.hematologylibrary.org/cgi/reprint/70/1/227).
2. Campbell H, Carothers A D, Rudan I, Hayward C, Biloglav Z, Barac L, Pericic M, Janicijevic B, Smolej-Narancic N, Polasek O, Kolcic I, Weber J L, Hastie N D, Rudan P, Wright A F. Effects of genome-wide heterozygosity on a range of biomedically relevant human quantitative traits. Hum Mol Genet 2007; 16(2):233-41 (full-text: http://hmg.oxfordjournals.org/cgi/content/full/16/2/233).
3. Canalle R, Burim R V, Tone L G, Takahashi C S. Genetic polymorphisms and susceptibility to childhood acute lymphoblastic leukemia. Environ Mol Mutagen 2004; 43(2):100-9.
4. Carpentier N A, Jeannet M. Increased HLA-DR compatibility between patients with acute myeloid leukemia and their parents: implication for bone marrow transplantation. Transplantation Proceedings 1987; 19: 2644-2645.
5. Chokkalingam A P & Buffler P A. Genetic susceptibility to childhood leukaemia. Radiat Prot Dosimetry 2008; 132(2):119-29.
6. Denic S, Nagelkerke N, Nicholls M G. Germline genomic homozygosity and cancer risk. JAMA 2008; 300(2):169-70.
7. Doherty P C, Zinkernagel R M. Enhanced immunological surveillance in mice heterozygous at the H-2 gene complex. Nature 1975; 256(5512):50-2.
8. Dorak M T, Lawson T, Machulla H K, Darke C, Mills I U, Burnett A K. Unravelling an HLA-DR association in childhood acute lymphoblastic leukemia. Blood 1999a;94(2):694-700 (Full-text: http://bloodjournal.hematologylibrary.org/cgi/content/abstract/94/2/694).
9. Dorak M T, Burnett A K, Worwood M, Sproul A M, Gibson B E. The C282Y mutation of HFE is another male-specific risk factor for childhood acute lymphoblastic leukemia. Blood 1999b; 94(11):3957 (Full-text: http://bloodjournal.hematologylibrary.org/cgi/content/full/94/11/3957).
10. Dorak M T, Oguz F S, Yalman N, Diler A S, Kalayoglu S, Anak S, Sargin D, Carin M N. A male-specific increase in the HLA-DRB4 (DR53) frequency in high-risk childhood ALL. Leukemia Research 2002a;26(7):651-8.
11. Dorak M T, Lawson T, Machulla H K, Mills K I, Burnett A K. Increased heterozygosity for MHC class II lineages in newborn males. Genes Immun 2002b;3(5):263-9 (Full-text: http://www.nature.com/gene/journal/v3/n5/full/6363862a.html).
12. Dorak M T, Burnett A K, Worwood M. HFE gene mutations in susceptibility to childhood leukemia: HuGE review. Genet Med 2005; 7(3):159-68 (Full-text: http://www.cdc.gov/genomics/hugenet/reviews/leukemia.htm).
13. Dorak M T, Pearce M S, Hammal D M, McNally R J, Parker L. Examination of gender effect in birth weight and miscarriage associations with childhood cancer (United Kingdom). Cancer Causes Control 2007; 18(2):219-28.
14. Lilly F, Boyse E A, Old L J. Genetic basis of susceptibility to viral leukemogenesis. Lancet 1964; ii: 1207-1209.
15. Linet M S, Wacholder S, Zahm S H. Interpreting epidemiologic research: lessons from studies of childhood cancer. Pediatrics 2003; 112(1 Pt 2):218-32.
16. Shannon K. Genetic predispositions and childhood cancer. Environ Health Perspect 1998; 106 Suppl 3: 801-6.
17. Sinnett D, Labuda D, Krajinovic M. Challenges identifying genetic determinants of pediatric cancers—the childhood leukemia experience. Fam Cancer 2006; 5(1):35-47.
18. Von Fliedner V E, Sultan-Khan Z, Jeannet M. HLA-DRw antigens associated with acute leukemia. Tissue Antigens 1980; 16: 399-404.
19. Von Fliedner V E, Merica H, Jeannet M, et al. Evidence for HLA-linked susceptibility factors in childhood leukemia. Human Immunology 1983; 8: 183-193.

TABLE 1
List of Genes and SNP Evaluated for Their Predictive Value as
Markers for Childhood Leukemia

| Gene and SNP Position | SNP ID | Chromosome position |
|---|---|---|
| HFE2 (HJV)-5′FLANK | rs4970862 | chr1:144132834 |
| HFE2 (HJV)-3′FLANK | rs1535921 | chr1:144129407 |
| IL10 | rs1800872 | chr1:205013030 |
| IL10 | rs1800896 | chr1:205013520 |
| ACP1-Ex3 | rs11553746 | chr2:262203 |
| ACP1-3′FLANK | rs12714402 | chr2:262926 |
| ACP1-IVS3 | rs7419262 | chr2:263621 |
| ACP1-3′UTR | rs6708541 | chr2:272736 |
| PKR (EIF2AK2)-IVS2 | rs2270414 | chr2:37216952 |
| PKR (EIF2AK2)-IVS1 | rs12712526 | chr2:37224339 |
| PKR (EIF2AK2)-5′UTR | rs2254958 | chr2:37229795 |
| RRM2-5′UTR | rs1130609 | chr2:10180371 |
| IL1B-5′FLANK | rs1143627 | chr2:113310858 |
| STEAP3-5′UTR | rs1562256 | chr2:119687643 |
| STEAP3-IVS1 | rs865688 | chr2:119699720 |
| STEAP3-IVS1 | rs865108 | chr2:119702854 |
| LCT-3′UTR | rs1042712 | chr2:136262314 |
| CYBRD1-IVS1 | rs960748 | chr2:172088182 |
| CYBRD1-IVS1 | rs6759240 | chr2:172089044 |
| CYBRD1-G266A-Ex4 | rs10455 | chr2:172119519 |
| SLC40A1-V221V | rs2304704 | chr2:190138422 |
| SLC40A1-IVS5 | rs4145237 | chr2:190140522 |
| SLC40A1-IVS2 | rs1439812 | chr2:190148793 |
| SLC40A1-IVS2 | rs1439814 | chr2:190151138 |
| SLC40A1-IVS7 | rs1439816 | chr2:190152875 |
| CTLA4-T17A-Ex1 | rs231775 | chr2:204440959 |
| SLC11A1-5′UTR | rs1059823 | chr2:218968088 |
| LTF-N541N-Ex13 | rs1042073 | chr3:46459968 |
| LTF-IVS12 | rs6441995 | chr3:46471344 |
| TF-P589S-Ex15 | rs1049296 | chr3:134977044 |
| TF-L524L-Ex13 | rs8649 | chr3:134969648 |
| TF-5′UTR | rs1130459 | chr3:134947973 |
| TF-5′FLANK | rs4481157 | chr3:134947374 |
| TF-5′FLANK | rs16840812 | chr3:134945497 |
| CP-E543D-Ex9 | rs701753 | chr3:150398925 |
| CP-IVS1 | rs7652826 | chr3:150421640 |
| TFRC-S142G-Ex4 | rs3817672 | chr3:197285208 |
| TFRC-5′UTR | rs11915082 | chr3:197293536 |
| EGF | rs2237051 | chr4:111120647 |
| EGF | rs4444903 | chr4:111053559 |
| NFKB1-IVS6 | rs4648022 | chr4:103715475 |
| IRF4 | rs2797301 | chr6:327111 |
| IRF4 | rs4985288 | 327246 |
| IRF4 | rs9405192 | 327537 |
| IRF4-5′FLANK | rs1033180 | 328546 |
| IRF4-IVS4 | rs12203592 | 341321 |
| IRF4 | rs3778607 | 348799 |
| IRF4 | rs2001508 | 349632 |
| IRF4 | rs7768807 | 353246 |
| IRF4 | rs1877175 | 355493 |
| IRF4-3′UTR | rs9392502 | 355608 |
| IRF4-3′UTR | rs872071 | 356064 |
| IRF4 | rs11242865 | 356954 |
| IRF4 | rs7757906 | 357741 |
| IRF4 | rs9378805 | 362727 |
| BMP6-5′UTR | rs12198986 | 7665058 |
| BMP6-IVS1 | rs7753111 | 7675943 |
| BMP6-V368V | rs17557 | 7807630 |
| BMP6-IVS4 | rs1225932 | 7820754 |
| Ch6:9559183 | rs10484246 | 9559183 |
| EDN1-5′FLANK | rs3756863 | 12397016 |
| EDN1-IVS2 | rs1476046 | 12401207 |
| EDN1-IVS4 | rs1626492 | 12403489 |
| EDN1-K198N-Ex5 | rs5370 | 12404241 |
| EDN1-3′FLANK | rs4714383 | 12405468 |
| EDN1-3′FLANK | rs4714384 | 12405839 |
| Ch6:20099022 | rs965036 | 20099022 |
| CDKAL1 | rs6908425 | 20836710 |
| PRL | rs4712652 | 22186594 |
| PRL-promoter | rs1341239 | 22412183 |
| SLC17A3 | rs1165165 | 25970445 |
| HIST1H4A-5′FLANK | rs9467664 | 26129792 |
| HIST1H3B-3′UTR | rs2213284 | 26139847 |
| HIST11H2AB-L97L | rs2230655 | 26141485 |
| HIST1H1C-P195P | rs8384 | 26164051 |
| HIST1H1C-S36S | rs10425 | 26164528 |
| HIST1H1C 5′FLANK | rs9393682 | 26165029 |
| HIST1H1C-5′FLANK | rs9358903 | 26169928 |
| HIST1H1C-5′FLANK | rs807212 | 26173600 |
| HFE-HIST1H1C-intergenic | rs2050947 | 26178058 |
| HFE-5′FLANK | rs4529296 | 26191114 |
| HFE-5′FLANK | rs1800702 | 26194442 |
| HFE-5′FLANK | rs2794720 | 26195181 |
| HFE-5′FLANK | rs2794719 | 26196869 |
| HFE-IVS1 | rs9366637 | 26197077 |
| HFE-H63D-Ex2 | rs1799945 | 26199158 |
| HFE-S65C-Ex2 | rs1800730 | 26199164 |
| HFE-IVS2 | rs2071303 | 26199315 |
| HFE-C282Y-Ex4 | rs1800562 | 26201120 |
| HFE-IVS5 | rs2858996 | 26202005 |
| HFE-3′FLANK | rs707889 | 26203910 |
| HIST1H4C-5′ & | rs12346 | 26205025 |
| HFE-3′FLANK | | |
| HIST1H4C-5′ & | rs17596719 | 26205173 |
| HFE-3′FLANK | | |
| HIST1H4C-5′FLANK | rs198853 | 26212075 |
| HIST1H4C-I35I-Ex1 | rs2229768 | 26212259 |
| HIST1H1T-Q178K-Ex1 | rs198845 | 26215769 |
| HIST1H1T-V14L-Ex1 | rs198844 | 26216261 |
| UBD-C160S-Ex2 | rs8337 | 29631655 |
| UBD-T68C-Ex2 | rs2076485 | 29631931 |
| UBD-IVS1 | rs2534790 | 29632147 |
| UBD-5′FLANK | rs1233405 | 29637733 |
| HLA-G-5′FLANK | rs1736939 | 29901364 |
| HLA-G-3′UTR indel | rs1704 | 29906560 |
| (aka rs16375) | | |
| ZNRD1 | rs9261269 | 30138093 |
| HLA-E-3′FLANK | rs1264456 | 30570063 |
| MDC1-A1657A | rs28986317 | 30779968 |
| MDC1-R268K | rs9262152 | 30788895 |
| MDC1-C179T | rs28986464 | 30789456 |
| IER3-3′UTR | rs10947089 | 30818114 |
| DDR1 | rs1264328 | 30958121 |
| DDR1 | rs1264327 | 30958561 |
| DDR1 | rs1264323 | 30963886 |
| DDR1 | rs1049623 | 30972808 |
| GTF2H4-5′FLANK | rs3909130 | 30982144 |
| GTF2H4 | rs1264309 | 30983878 |
| GTF2H4-IVS11 | rs1264307 | 30988736 |
| TCF19-5′FLANK | rs1265086 | 31217861 |
| TCF19-IVS1 | rs1150765 | 31235541 |
| TCF19-IVS1 | rs6905862 | 31235581 |
| TCF19-P219P-Ex2 | rs2073722 | 31237621 |
| POU5F1-IVS4 | rs2394882 | 31240628 |
| POU5F1-IVS1-Ex1-M1R | rs3130932 | 31241922 |
| HLA class I | rs3873375 | 31359339 |
| HLA-C-5′FLANK | rs9264942 | 31382359 |
| MICA-V152M-Ex3 | rs1051792 | 31486956 |
| MICA STR | UniSTS:464273 | 31488069 |
| NFKBIL1-promoter; htSNP | rs2523502 | 31621843 |
| NFKBLL1-promoter; htSNP | rs2071592 | 31623319 |
| NFKBIL1-3′end; htSNP | rs2857605 | 31632830 |
| NFKBIL1-3′end; htSNP | rs2239707 | 31633298 |
| NFKBIL1-3′FLANK; htSNP | rs2516390 | 31637862 |
| LTA-IVS1 | rs909253 | 31648292 |
| TNF-promoter-857 | rs1799724 | 31650461 |
| TNF-promoter-238 | rs361525 | 31651080 |
| NCR3-3′FLANK | rs2256965 | 31663109 |
| NCR3-3′UTR | rs1052248 | 31664560 |
| NCR3-5′UTR | rs986475 | 31664688 |
| NCR3/AIF1/BAT2 region | rs2844479 | 31680935 |
| AIF1-IVS1 | rs2844475 | 31691134 |
| AIF1-5′UTR-IVS3 | rs2259571 | 31691806 |
| AIF1-R15W-IVS4 | rs2269475 | 31691910 |
| AIF-5′FLANK | rs2857694 | 31695849 |
| BAT2-IVS7 | rs2260000 | 31701455 |
| BAT2-IVS12 | rs3132450 | 31704117 |
| BAT3-3′FLANK | rs2736155 | 31713178 |
| BAT3-IVS14 | rs1077393 | 31718508 |
| BAT3-IVS12 | rs2077102 | 31719819 |
| BAT3-IVS6 | rs805303 | 31724345 |
| CLIC1 | rs2272592 | 31806331 |
| CLIC1 | rs3131383 | 31812273 |
| MSH5 | rs2075789 | 31816307 |
| MSH5 | rs28381349 | 31817024 |
| MSH5 | rs3117572 | 31825671 |
| MSH5 | rs3131379 | 31829012 |
| MSH5 | rs3131378 | 31833264 |
| MSH5 | rs707939 | 31834667 |
| MSH5 | rs3115672 | 31835876 |
| MSH5-Q716Q-Ex22 | rs707938 | 31837338 |
| MSH5-P786S-Ex24 | rs1802127 | 31837904 |
| HSPA1L-G602K | rs2075800 | 31885925 |
| HSPA1L-T493M | rs2227956 | 31886251 |
| HSPA1A-5′UTR (−27) | rs1043618 | 31891486 |
| HSPA1B-5′FLANK (−1136) | rs2763979 | 31902571 |
| HSPA1B-Q351Q | rs1061581 | 31904759 |
| CFB-R32W | rs12614 | 32022158 |
| CFB-IVS14 | rs1270942 | 32026839 |
| SKIV2L-IVS2 | rs440454 | 32035321 |
| SKIV2L-Q151R-Ex5 | rs438999 | 32036285 |
| SKIV2L-IVS6 | rs2280774 | 32036670 |
| SKIV2L-IVS6 | rs419788 | 32036778 |
| SKIV2L-Y1067Y-Ex26 | rs410851 | 32044647 |
| CYP21A2-R103K | rs6474 | 32114865 |
| CYP21A2-V282L | rs6471 | 32115866 |
| TNXB-H1248R | rs185819 | 32158045 |
| TNXB-3′FLANK | rs3130342 | 32188124 |
| TNXB-3′UTR | rs8283 | 32191278 |
| EGFL8-R86K | rs3096697 | 32242488 |
| EGFL8-3′UTR | rs1061808 | 32244525 |
| PBX2-3′FLANK | rs1800684 | 32259972 |
| PBX2-IVS4 | rs204993 | 32263559 |
| NOTCH4-IVS11 | rs3134799 | 32292199 |
| NOTCH4-S244L-Ex4 | rs8192585 | 32296801 |
| NOTCH4-K117Q-Ex3 | rs915894 | 32298368 |
| NOTCH4-IVS1 | rs396960 | 32299559 |
| NOTCH4-5′FLANK | rs3096702 | 32300309 |
| NOTCH4-5′FLANK | rs3096690 | 32302608 |
| C6orf10-K400Q-Ex23 | rs7775397 | 32369230 |
| C6orf10-IVS6 | rs1265758 | 32431507 |
| C6orf10 | rs9268428 | 32452951 |
| C6orf10 | rs1980495 | 32454772 |
| BTNL2 | rs3129953 | 32469799 |
| BTNL2 | rs2076530 | 32471794 |
| BTNL2-Q350Q | rs9268480 | 32471822 |
| DRA-5′UTR | rs14004 | 32515687 |
| HLA-DRA-V16L-Ex1 | rs16822586 | 32515751 |
| HLA-DRA-I134I-Ex3 | rs8084 | 32519013 |
| HLA-DRA-L242V-Ex4 | rs7192 | 32519624 |
| HLA-DRA-3′UTR | rs7194 | 32520458 |
| HLA-DRA-3′FLANK | rs3135388 | 32521029 |
| BTNL2 | rs2076525 | 32541145 |
| HLA-DQA1 | rs2395185 | 32541145 |
| HLA DRB1-DQA1 | rs660895 | 32685358 |
| HLA-DQA1-3′UTR | rs1142316 | 32686523 |
| HLA-DRB1-DQA1 region | rs3135005 | 32693997 |
| HLA-DRB1-DQA1 region | rs9271366 | 32694832 |
| HLA-DRB1-DQA1 region | rs2395225 | 32698602 |
| HLA-DRB1-DQA1 region | rs9271586 | 32698877 |
| HLA-DRB1-DQA1 region | rs3129763 | 32698903 |
| HLA-DRB1-DQA1 region | rs17599077 | 32699036 |
| HLA-DQA1-IVS1 | rs17426593 | 32716055 |
| HLA-DQA1-IVS2 | rs9272723 | 32717405 |
| HLA-DQA1 | rs2157051 | 32766602 |
| HLA-DQA2 | rs2227128 | 32819378 |
| HLA-DQB2 | rs1573649 | 32839236 |
| TAP2 | rs241453 | 32904204 |
| BRD2-5′FLANK | rs206786 | 33043157 |
| BRD2-IVS3 | rs635688 | 33051129 |
| BRD2-IVS7 | rs11908 | 33052724 |
| BRD2-3′UTR | rs1049414 | 33056585 |
| RXRB-F384F-Ex7 | rs6531 | 33271429 |
| RXRB-IVS3 | rs2076310 | 33274012 |
| ZIP7/SLC39A7 | rs41266701 | 33277817 |
| (RXRB-5′FLANK) | | |
| ZIP7/SLC39A7 | rs1547387 | 33277873 |
| (RXRB-5′FLANK) | | |
| HSD17B8-IVS2 | rs365339 | 33280883 |
| (RXRB-5′FLANK) | | |
| HSD17B8-IVS6 | rs439205 | 33281820 |
| (RXRB-5′FLANK) | | |
| HSD17B8-IVS7 | rs383711 | 33281976 |
| (RXRB-5′FLANK) | | |
| HSD17B8-3′FLANK | rs421446 | 33282761 |
| DAXX-IVS4 | rs2239839 | 33396053 |
| DAXX-Y379Y-Ex4 | rs1059231 | 33396249 |
| DAXX-IVS1 | rs2073524 | 33398525 |
| CDKN1A | rs733590 | 36753181 |
| CDKN1A | rs2395655 | 36753674 |
| CDKN1A | rs3176352 | 36760317 |
| CDKN1A | rs12207548 | 36764234 |
| CDKN1A | rs7767246 | 36767193 |
| PIM1 | rs1757000 | 37243144 |
| VEGFA-promoter | rs699947 | 43844367 |
| VEGFA-promoter | rs1005230 | 43844474 |
| VEGFA-promoter | rs1570360 | 43845808 |
| VEGFA-3′UTR-Exon 8 | rs3025039 | 43860514 |
| VEGFA-3′UTR-Exon 8 | rs10434 | chr6:43861190 |
| IL6-5′UTR | rs1800796 | chr7:22732771 |
| IL6-5′UTR | rs1800797 | chr7:22732746 |
| IGFBP3-5′FLANK | rs2854744 | chr7:45927600 |
| TFR2-IVS17 | rs10247962 | chr7:100057865 |
| TFR2-IVS3 | rs7385804 | chr7:100073906 |
| SLC39A14-5′FLANK | rs4872476 | chr8:22266179 |
| SLC39A14-5′FLANK | rs11136002 | chr8:22273027 |
| SLC39A14-L33C | rs896378 | chr8:22318266 |
| SLC39A14-IVS8 | rs10101909 | chr8:22332985 |
| SLC39A4-T332A-Ex5 | rs2272662 | chr8:145610534 |
| LCN2 | rs10819368 | chr9:129946167 |
| LCN2 | rs878400 | chr9:129947865 |
| LCN2 | rs10987900 | chr9:129958277 |
| H19 | rs217727 | chr11:1973484 |
| RRM1-IVS2 | rs232054 | chr11:4080003 |
| KLRK1 3′FLANK | rs10772266 | chr12:10397436 |
| KLRK1 3′UTR-Ex10 | rs1049174 | chr12:10416632 |
| KLRK1-IVS1 | rs2617160 | chr12:10436864 |
| KLRK1-IVS1 | rs2246809 | chr12:10448311 |
| KLRC4-IVS3 | rs2734565 | chr12:10451858 |
| KLRC4-S104N-Ex3 | rs2617170 | chr12:10452224 |
| KLRC4-IVS2 | rs2617171 | chr12:10452546 |
| KLRC4-S29I-Ex1 | rs1841958 | chr12:10453356 |
| KLRC1-5′FLANK | rs1983526 | chr12:10499280 |
| KLRC1-5′FLANK | rs2900421 | chr12:10513314 |
| SLC11A2-3′FLANK | rs853235 | chr12:49662236 |
| SLC11A2-IVS4 | rs224589 | chr12:49685317 |
| SLC11A2-IVS1 | rs422982 | chr12:49692621 |
| SLC11A2-IVS1 | rs407135 | chr12:49697620 |
| SLC11A2-IVS1 | rs224575 | chr12:49705888 |
| IFNG-3′FLANK | rs2069727 | chr12:66834490 |
| IFNG-IVS1 | rs2430561 | chr12:66838787 |
| IFNG | rs2069705 | chr12:66841278 |
| MDM2-IVS1 (aka SNP309) | rs2279744 | chr12:67488847 |
| IGF1-3′ UTR-Ex4 | rs6220 | chr12:101318645 |
| IGF1-IVS3 | rs1520220 | chr12:101320652 |
| BRCA2-N372H-Ex10 | rs144848 | chr13:31804729 |
| IREB2 | rs2656070 | chr15:76517307 |
| IGF1R-E1043E-Ex16 | rs2229765 | chr15:97295748 |
| HP-5′UTR | rs9924964 | chr16:70643062 |
| HP-5′UTR | rs7203426 | chr16:70644056 |
| HP-IVS1 | rs2070937 | chr16:70647241 |
| TP53-R72P-Ex4 | rs1042522 | chr17:7520197 |
| BRIP1-IVS4 | rs4968451 | chr17:57282089 |
| HAMP-5′FLANK | rs1882694 | chr19:40463222 |
| HAMP-5′FLANK | rs10414846 | chr19:40464311 |
| HAMP-IVS1 | rs8101606 | ch19:40466396 |
| HAMP-IVS1 | rs7251432 | chr19:40467281 |
| BMP2-3′FLANK | rs235756 | chr20:6715111 |
| LIF-3′UTR | rs929271 | chr22:28968226 |
| LIF-IVS2 | rs737921 | chr22:28970214 |
| LIF-IVS2 | rs929273 | chr22:28970595 |
| LIF-5′FLANK | rs2267153 | chr22:28973609 |
| LIF-5′FLANK | rs3761427 | chr22:28974826 |
| LIF-5′FLANK | rs9606708 | chr22:28976126 |
| HMOX1-5′FLANK | rs5755709 | chr22:34096930 |
| HMOX1-5′FLANK | rs735267 | chr22:34098057 |
| HMOX1-D7H-Ex1 | rs2071747 | chr22:34107185 |
| HMOX1-IVS1 | rs2071748 | chr22:34107618 |
| HMOX1-IVS2 | rs9607267 | chr22:34111207 |
| HMOX1-IVS3 | rs2071749 | chr22:34113413 |
| HMOX1-3′UTR | rs743811 | chr22:34122974 |
| TMPRSS6-Y739Y-Ex17 | rs2235321 | chr22:35792872 |
| TMPRSS6-V736A-Ex17 | rs855791 | chr22:35792882 |
| TMPRSS6-D511D-Ex13 | rs4820268 | chr22:35799537 |
| TMPRSS6-IVS2 | rs733655 | chr22:35824997 |
| TMPRSS6-5′UTR | rs5756515 | chr22:35829638 |
| HEPH-5′FLANK | rs5919015 | X chr:65299410 |
| HEPH-IVS18 | rs4827365 | X chr:65397067 |
| HEPH-IVS18 | rs2198868 | X chr:65399577 |

TABLE 2
Characteristics of single nucleotide polymorphisms and other polymorphisms found to be
predictors of childhood leukemia in univariable statistical association tests

| Gene and SNP | | | Position in |
|---|---|---|---|
| Position | SNP ID | Alternative Name | Gene/Change |
| IL10 | rs1800872 | no alternative name | 3′ flanking region, |
| | | | C > A |
| ACP1 | rs12714402 | NT_022327.14:g.262926A > G | 3′ flanking region, |
| | | | G > A |
| PKR (EIF2AK2) | rs2270414 | NT_022184.14:g.16191865G > A | intron 2, C > T |
| PKR (EIF2AK2) | rs12712526 | NT_022184.14:g.16199248A > G | intron 1, A > G |
| PKR (EIF2AK2) | rs2254958 | NT_022184.14:g.16192224G > A | 5′ UTR, C > T |
| STEAP3 | rs865688 | NT_022135.15:g.8691172G > A | intron 1, A > G |
| SLC40A1 | rs1439812 | NT_005403.16:g.40649965T > G | intron 2, T > G |
| CTLA4 | rs231775 | NT_005403.16:g.54942131A > G | exon 1, A > G |
| | | | (T17A) |
| TF | rs1049296 | NT_005612.15:g.39989499C > T | exon 15, C > T |
| TF | rs8649 | NT_005612.15:g.39982103G > C | exon 13, G > C |
| TF | rs1130459 | NT_005612.15:g.39960429A > G | 5′ UTR, G > A |
| TF | rs4481157 | NT_005612.15:g.39959830G > A | 5′ flanking region, |
| | | | G > A |
| LTF | rs1042073 | NT_022517.17:g.46424967G > A | exon 13, C > T |
| | | | (N541N) |
| EGF | rs4444903 | NT_016354.18:g.35382256A > G | A > G |
| NFKB1 | rs4648022 | NT_016354.18:g.28044172C > T | intron 6, C > T |
| IRF4 | rs12203592 | NT_034880.3:g.336321C > T | intron 4, C > T |
| BMP6 | rs17557 | NT_034880.3:g.7802629G > C | exon 4, G > C |
| | | | (V368V) |
| EDN1 | rs5370 | NT_007592.14:g.3154512G > T | exon 5, G > T |
| | | | (K198N) |
| HFE | rs807212 | no alternative name | 5′ flanking region, |
| | | | C > T |
| HFE | rs1800562 | NT_007592.14:g.16951391G > A | exon 4, G > A |
| | | | (C282Y) |
| HFE | rs17596719 | no alternative name | 3′ flanking region, |
| | | | G > A |
| HIST1H1T | rs198844 | NT_007592.14:g.16966532C > G | exon 1, C > G |
| | | | (L14V) |
| UBD | rs2534790 | NT_007592.14:g.20382419G > T | intron 1, C > A |
| HLA-G | rs1736939 | no alternative name | 5′ flanking region, |
| | | | C > T |
| HLA-G | rs1704 | NT_007592.14:g.20656832_20656 | 3′UTR, indel |
| | | 833insC | |
| ZNRD1 | rs9261269 | NT_007592.14:g.20888365A > G | intron 4, G > A |
| HLA-E | rs1264456 | no alternative name | 3′ flanking region, |
| | | | C > T |
| DDR1 | rs1264328 | NT_007592.14:g.21708393A > G | 5′ flanking region, |
| | | | T > C |
| DDR1 | rs1264323 | NT_007592.14:g.21714158G > A | intron 3, C > T |
| DDR1 | rs1049623 | NT_007592.14:g.21723079T > C | exon 15, A > G |
| | | | (V599V) |
| HLA-C | rs9264942 | no alternative name | 5′ flanking region, |
| | | | T > C |
| MICA | rs1051792 | NT_007592.14:g.22237227G > A | exon 3, G > A |
| | | | (V152M) |
| BAT3 | rs2077102 | NT_007592.14:g.22470091C > A | intron 12, G > T |
| HSPA1B | rs1061581 | no alternative name | exon 1, A > G |
| | | | (Q351Q) |
| SKIV2L | rs419788 | NT_007592.14:g.22787050T > C | intron 6, G > A |
| NOTCH4 | rs3096702 | NT_007592.14:g.23050581A > G | 3′ flanking region, |
| | | | T > C |
| BTNL2 | rs9268480 | NT_007592.14:g.23222093C > T | exon 5, C > T |
| | | | (Q350Q) |
| HLA-DRA | rs7192 | NT_007592.14:g.23269895T > G | exon 4, G > T |
| | | | (L242V) |
| HLA-DRA | rs3135388 | NT_007592.14:g.23271301A > G | 3′ flanking region, |
| | | | C > T |
| HLA-DQA1 | rs1142316 | no alternative name | 3′UTR, A > C |
| HLA-DRB1-DQA1 | rs2395225 | no alternative name | T > C |
| region | | | |
| HLA-DRB1-DQA1 | rs9271586 | no alternative name | T > G |
| region | | | |
| RXRB | rs6531 | NT_007592.14:g.24021700G > A | exon 7, T > C (F384F) |
| RXRB | rs2076310 | NT_007592.14:g.24024284A > G | intron 3, T > C |
| HSD17B8/RXRB | rs365339 | NT_007592.14:g.24031155T > C | intron 2, G > A |
| HSD17B8/RXRB | rs421446 | NT_007592.14:g.24033033A > G | 5′ flanking region, |
| | | | T > C |
| DAXX | rs2239839 | NT_007592.14:g.24146325C > A | intron 4, G > T |
| DAXX | rs1059231 | NT_007592.14:g.24146521A > G | exon 4, T > C |
| | | | (Y379Y) |
| DAXX | rs2073524 | NT_007592.14:g.24148797T > A | intron 1, T > A |
| VEGFA | rs1570360 | NT_007592.14:g.34596080A > G | promoter |
| IL6 | rs1800797 | NT_007819.16:g.22255179A > G | 5′ UTR, G > A |
| TFR2 | rs10247962 | NT_007933.14:g.25454205G > A | intron 17, A > G |
| SLC39A14 | rs11136002 | no alternative name | 5′ flanking region, |
| SLC39A4 | rs2272662 | NT_037704.4:g.207137T > C | exon 5, G > A |
| | | | (T332A) |
| LCN2 | rs878400 | no alternative name | T > C |
| KLRK1 Region | rs1049174 | NT_009714.16:g.3284339G > C | exon 10, 3′UTR, G > C |
| (KLRK1) | | | |
| KLRK1 Region | rs2617160 | NT_009714.16:g.3304571A > T | intron 1, A > T |
| (KLRK1) | | | |
| KLRK1 Region | rs2734565 | NT_009714.16:g.3319565C > T | intron 3, A > G |
| (KLRC4) | | | |
| KLRK1 Region | rs2617170 | NT_009714.16:g.3319930T > C | exon 3, C > T, |
| (KLRC4) | | | (S104N) |
| KLRK1 Region | rs2617171 | NT_009714.16:g.3320253C > G | intron 2, C > G |
| (KLRC4) | | | |
| KLRK1 Region | rs1841958 | NT_009714.16:g.3321062A > C | exon 1, C > A (S291I) |
| (KLRC4) | | | |
| KLRK1 Region | rs1983526 | no alternative name | 5′ flanking region, |
| (KLRC1) | | | C > G |
| SLC11A2 | rs224589 | NT_029419.11:g.13542356T > G | intron 4, C > A |
| IFNG | rs2069727 | NT_029419.11:g.30691529T > C | 3′ flanking region, |
| | | | A > G |
| TP53 | rs1042522 | NT_010718.15:g.7176820G > C | exon 4, C > G (R72P) |
| LIF | rs929271 | NT_011520.11:g.10028795T > G | 3′UTR, T > G |
| LIF | rs737921 | NT_011520.11:g.10030783G > A | intron 2, G > A |
| LIF | rs929273 | NT_011520.11:g.10031164G > A | intron 2, G > A |
| LIF | rs2267153 | no alternative name | 3′ flanking region, |
| | | | C > G |
| HMOX1 | rs2071748 | NT_011520.11:g.15168187G > A | intron 1, G > A |
| HMOX1 | rs5755709 | NT_011520.11:g.15157499G > A | 5 flanking region, |
| | | | G > A |
| TMPRSS6 | rs855791 | NT_011520.11:g.16853450A > G | exon 17, C > T |
| | | | (V736A) |
| TMPRSS6 | rs733655 | NT_011520.11:g.16885566T > C | intron 2, T > C |

TABLE 3
Individual predictive value of the single nucleotide polymorphisms and other
polymorphisms or their combinations in females

| | | Univariable odds ratio (95% |
|---|---|---|
| Gene/SNP/Genotype | Group* | CI) and P value |
| BMP6 rs17557/heterozygosity | HLA | 0.50 (0.24 to 1.00); P = 0.05 |
| UBD rs2534790/homozygosity | HLA | 2.72 (1.02 to 7.48); P = 0.05 |
| HLA-G rs1736939/heterozygosity | HLA | 0.44 (0.22 to 0.87); P = 0.02 |
| HLA-G rs1704/heterozygosity | | |
| ZNRD1 rs9261269/heterozygosity | HLA | 0.30 (0.10 to 0.89); P = 0.03 |
| DDR1 rs1264328/heterozygosity | HLA | 0.50 (0.25 to 1.00); P = 0.05 |
| DDR1 rs1264323/heterozygosity | | |
| DDR1 rs1049623/heterozygosity | | |
| HLA-C rs9264942/variant allele positive | HLA | 0.45 (0.23 to 0.86); P = 0.015 |
| SKIV2L rs419788/variant allele positive | HLA | 2.11 (1.07 to 4.15); P = 0.03 |
| HLA-DRA rs3135388/variant allele positive | HLA | 2.87 (1.49 to 5.50); P = 0.002 |
| DAXX rs2073524/homozygosity | HLA | 3.36 (1.32 to 8.50); P = 0.01 |
| DAXX rs1059231/homozygosity | | |
| DAXX rs2239839/wildtype homozygosity | | |
| DAXX rs2239839/homozygosity | HLA | 2.24 (1.00 to 5.02); P = 0.05 |
| STEAP3 rs865688/variant allele positive | IRG | 0.46 (0.24 to 0.88); P = 0.02 |
| SLC40A1 rs1439812/heterozygosity | IRG | 0.41 (0.19 to 0.87); P = 0.02 |
| SLC40A1 rs1439812/homozygosity | IRG | 2.77 (1.03 to 7.47); P = 0.04 |
| HFE rs807212/heterozygosity | IRG | 0.44 (0.22 to 0.90); P = 0.02 |
| TFR2 rs10247962/homozygosity | IRG | 7.50 (2.03 to 27.8); P = 0.003 |
| LCN2 rs878400/heterozygosity | IRG | 0.45 (0.22 to 0.93); P = 0.03 |
| SLC11A2 rs224589/variant allele positive | IRG | 0.43 (0.19 to 0.98), P = 0.05 |
| HMOX1 rs2071748/homozygosity | IRG | 0.38 (0.14 to 1.00); P = 0.05 |
| HMOX1 rs5755709/homozygosity | IRG | 0.26 (0.07 to 0.93); P = 0.04 |
| IL10 rs1800872/heterozygosity | ISG | 0.52 (0.26 to 1.02); P = 0.06 |
| IL6 rs1800797/variant allele positive | ISG | 2.17 (1.07 to 4.43); P = 0.03 |
| EGF rs4444903/heterozygosity | OCR | 0.55 (0.29 to 1.03); P = 0.06 |
| EDN1 rs5370/variant allele positive | OCR | 0.36 (0.17 to 0.77); P = 0.008 |
| VEGFA rs1570360/homozygosity | OCR | 2.47 (1.03 to 5.89); P = 0.04 |
| TP53 rs1042522/homozygosity | OCR | 3.50 (1.40 to 8.76); P = 0.008 |

*HLA: HLA-complex genes; IRG: Iron regulatory genes; ISG: Immune surveillance genes; OCR: Other cancer-related genes

TABLE 4
Predictive value of the single nucleotide polymorphisms and
other polymorphisms or their combinations in the final
multivariable model in females

| | | | 95% CI of Odds | |
|---|---|---|---|---|
| Marker | Group* | Odds Ratio | Ratio | P value |
| DAXX rs2073524-rs1059231- | HLA | 3.62 | 1.13 to 11.5 | 0.03 |
| rs2239839 homozygosity | | | | |
| HLA-DRB1-DQA1 region | HLA | 0.26 | 0.08 to 0.82 | 0.02 |
| rs2395225-rs9271586 heterozygosity | | | | |
| HMOX1 rs2071748 homozygosity | IRG | 0.06 | 0.01 to 0.52 | 0.01 |
| TFR2 rs10247962 homozygosity | IRG | 99.8 | 5.21 to 1913.6 | 0.002 |
| IL10 rs1800872 heterozygosity | ISG | 0.30 | 0.12 to 0.76 | 0.01 |
| TP53 rs1042522 homozygosity | OCR | 5.05 | 1.48 to 17.2 | 0.01 |
| EDN1 rs5370 variant allele positivity | OCR | 0.22 | 0.09 to 0.56 | 0.002 |
| | | Odds | 95% CI of Odds | |
| Number of Markers Possessed | Group* | Ratio** | Ratio | P value |
| 0, 1, 2, 3 | Any | 1.0 | n/a | n/a |
| | | (Reference) | | |
| 4 | Any | 0.27 | 0.13 to 0.56 | 0.0004 |
| 5, 6, 7 | Any | 0.03 | 0.01 to 0.24 | 0.002 |

*HLA: HLA-complex genes; IRG: Iron regulatory genes; ISG: Immune surveillance genes; OCR: Other cancer-related genes.
**To construct this cumulative model, all odds ratios (OR) are converted to the same direction. For example, OR = 0.27 in this model corresponds to 1/0.27 = 3.70 and OR = 0.03 means 33.3 times increased risk.

TABLE 5
Individual predictive value of the single nucleotide polymorphisms and other
polymorphisms or their combinations in males

| | | Univariable odds ratio (95% |
|---|---|---|
| Gene/SNP/Genotype | Group* | CI) and P value |
| NFKB1 rs4648022/heterozygosity | HLA | 0.20 (0.05 to 0.89); P = 0.03 |
| MICA_rs1051792 homozygosity | HLA | 2.26 (1.19 to 4.31); P = 0.01 |
| MICA STR allele 185bp (A5.1)/homozygosity | | |
| BAT3 rs2077102/heterozygosity | HLA | 0.38 (0.17 to 0.85); P = 0.02 |
| BAT3 rs2077102/variant allele positive | HLA | 0.39 (0.18 to 0.85); P = 0.02 |
| HSPA1B rs1061581/variant allele positive | HLA | 0.48 (0.26 to 0.88); P = 0.02 |
| HSPA1B rs1061581/wildtype homozygosity | HLA | 3.38 (1.21 to 9.43); P = 0.02 |
| BTNL2 rs9268480 homozygosity | | |
| HLA-DRA rs7192 wildtype homozygosity | | |
| HSPA1B rs1061581/homozygosity | HLA | 3.94 (1.64 to 9.47); P = 0.002 |
| HLA-DRA rs7192/homozygosity | | |
| HLA-DQA1 rs1142316/homozygosity | | |
| NOTCH4 rs3096702/homozygosity | HLA | 2.05 (1.0 to 4.05); P = 0.05 |
| HLA-DRB1-DQA1 region rs2395225/wildtype | HLA | 2.45 (1.24 to 4.83); P = 0.01 |
| homozygosity | | |
| HLA-DRB1-DQA1 region rs9271586/ | | |
| homozygosity | | |
| TF rs1049296/heterozygosity | IRG | 0.45 (0.23 to 0.91); P = 0.03 |
| TF rs1049296/variant allele positive | IRG | 0.52 (0.27 to 0.99); P = 0.05 |
| TF rs1049296 wildtype homozygosity | IRG | 0.29 (0.09 to 1.00); P = 0.05 |
| TF rs8649 wildtype homozygosity | | |
| TF rs1130459 wildtype homozygosity | | |
| TF rs4481157 homozygosity | | |
| LTF rs1042073/variant allele positive | IRG | 0.40 (0.22 to 0.75); P = 0.004 |
| HFE rs807212/heterozygosity | IRG | 0.42 (0.22 to 0.79); P = 0.007 |
| SLC39A14 rs11136002/heterozygosity | IRG | 0.42 (0.22 to 0.81); P = 0.01 |
| SLC39A4 rs2272662/homozygosity | IRG | 2.91 (1.42 to 5.95), P = 0.003 |
| LCN2 rs878400/heterozygosity | IRG | 0.52 (0.28 to 0.96); P = 0.04 |
| TMPRSS6 rs733655/homozygosity | IRG | 6.37 (1.80 to 22.6), P = 0.004 |
| TMPRSS6 rs855791/variant allele positive | IRG | 0.49 (0.26 to 0.90), P = 0.02 |
| IL10 rs1800872/heterozygosity | ISG | 0.45 (0.21 to 0.96); P = 0.04 |
| PKR rs2270414/wildtype homozygous | ISG | 0.45 (0.20 to 1.02); P = 0.06 |
| PKR rs12712526/wildtype homozygous | | |
| PKR rs2254958/wildtype homozygous | | |
| CTLA4 231775/homozygosity | ISG | 2.28 (1.06 to 4.68), P = 0.04 |
| IRF4 rs12203592/homozygosity | ISG | 4.36 (1.51 to 12.6); P = 0.007 |
| NKG2D rs1049174/wildtype homozygosity | ISG | 2.46 (0.98 to 6.18); P = 0.06 |
| NKG2D rs2617160/wildtype homozygosity | | |
| NKG2D rs2734565/wildtype homozygosity | | |
| NKG2D rs2617170/wildtype homozygosity | | |
| NKG2D rs2617171/wildtype homozygosity | | |
| NKG2D rs1841958/wildtype homozygosity | | |
| NKG2D rs1983526/wildtype homozygosity | | |
| IFNG rs2069727/variant allele positive | ISG | 0.53 (0.29 to 0.97); P = 0.04 |
| ACP1 rs12714402/homozygosity | OCR | 2.48 (1.09 to 5.65), P = 0.03 |
| TP53 rs1042522/homozygosity | OCR | 2.44 (0.94 to 6.29); P = 0.07 |

*HLA: HLA-complex genes; IRG: Iron regulatory genes; ISG: Immune surveillance genes; OCR: Other cancer-related genes

TABLE 6
Predictive value of the single nucleotide polymorphisms and other
polymorphisms or their combinations in the final multivariable
model in males

| | | Odds | 95% CI of Odds | |
|---|---|---|---|---|
| Marker | Group* | Ratio | Ratio | P value |
| HLA-DRB1-DQA1 region | HLA | 3.20 | 1.22 to 8.39 | 0.02 |
| rs2395225-rs9271586 homozygosity | | | | |
| HSPA1B rs1061581 variant allele | HLA | 0.36 | 0.15 to 0.88 | 0.03 |
| positivity | | | | |
| MICA rs1051792 and | HLA | 2.76 | 1.18 to 6.45 | 0.02 |
| MICA STR 185bp homozygosity | | | | |
| HFE 807212 variant allele positivity | IRG | 0.17 | 0.07 to 0.43 | <0.001 |
| TMPRSS7 rs733655 homozygosity | IRG | 32.9 | 2.68 to 404.0 | 0.006 |
| LTF rs1042073 variant allele | IRG | 0.34 | 0.14 to 0.81 | 0.02 |
| positivity | | | | |
| PKR rs2270414-rs12712526- | ISG | 0.13 | 0.02 to 0.74 | 0.02 |
| rs2254958 homozygosity | | | | |
| | | Odds | 95% CI of Odds | |
| Number of Markers Possessed | Group* | Ratio** | Ratio | P value |
| 0, 1, 2, 3 | Any | 1.0 | n/a | n/a |
| | | (Reference) | | |
| 4 | Any | 0.20 | 0.10 to 0.40 | <0.0001 |
| 5, 6, 7 | Any | 0.08 | 0.03 to 0.20 | <0.0001 |

*HLA: HLA-complex genes; IRG: Iron regulatory genes; ISG: Immune surveillance genes; OCR: Other cancer-related genes.
These odds ratios (OR) represent 1/0.20 = 5.0 times increased risk for possession of 4 SNP markers, and 1/0.08 = 12.5 times increased risk for possession of 5, 6 or 7 SNP markers.

TABLE 7
Single nucleotide polymorphisms found
to predict childhood leukemia risk

IL10 rs1800872:
TCAGCAAGTGCAGACTACTCTTACCCACTTCCCCCAAGCACAGTTGGGGT
GGGGGACAGCTGAAGAGGTGGAAACATGTGCCTGAGAATCCTAATGAAAT
CGGGGTAAAGGAGCCTGGAACACATCCTGTGACCCCGCCTGT
A/C
CTGTAGGAAGCCAGTCTCTGGAAAGTAAAATGGAAGGGCTGCTTGGGAAC
TTTGAGGATATTTAGCCCACCCCCTCATTTTTACTTGGGGAAACTAAGGC
CCAGAGACCTAAGGTGACTGCCTAAGTTAGCAAGGAGAAGTCTTGGGTAT
TCATCCC
ACP1 rs12714402:
AGTCACAATCAAATTCTGCAATTTCAATTGAAGATAACCTTGTCTTTATA
TTATGAATTAGAAGCTAAAGTTGATTTTTCTAAGAGTTCTTTATTTAAAT
GAAGTACTCTGGGACTGACCTTTTCGGAAATGGAATCTTC
G/A
TTGGTCAGGTGATTCAACATTTTTATACAATTTATCCATCCTCATCTCTT
CAGGATTTGCATACCTTGCCAGTTTCTACTGGCCATTGTTGAAAATACAT
TTATTTGGAGAAGTCCAAAGCCAAGGGGCTCATGGGGCTGTGAAGTCCTT
CTTGCTGCAT
PKR rs2270414:
AAACTTTAGC AGTTCTTCCA TCTGACTCAG GTTTGCTTCT
CTGGCGGTCT TCAGAATCAA CATCCACACT TCCGTGATTA
TCTGCGTGCA TTTTGGACAA AGCTTCCAAC CAGGTACAAG
CGGTCTTCCG AATTTTGCAC TCAGAAAAGT GGCATCATCT
AAGTCAATTA CATGCAAATT
C/T
TGGGGGGCTA GTTTTTTGTG TATGTTAAAT GGGTCACAAC
ACGACTTCTG TAAATCCTCA AATCTGTCAA TATAAATTTT
TATGTGATGA AAGCAAATTG TATTGTTCCT AGAAAGTGTC
CTTCCAGTTC TAAGTTGAAG TAAAAGCATG TCATTTGATG
ACAATTCTTG CAACATCTTA
PKR rs12712526:
GGGCAGAGCG GGGTTTCTTG TATAGGCAGG TTGTTTGAGG
AAGGCTGCTC TGATAAGCTG GCATGGGAAG CAGTGCAGGA
TAAGGGAGGG ATTTCCCCAT GCAGTTATCT GGGGAAGAAG
CTTTCCAGAA AGAAGAAACA
A/G
GCAGTGCAAA GGCCTAGAGG CTGGAGGATG CTTGGCTGTG
CACCAGGAAC AGCAAGGAAG CCAGCGTGGC CGGAGTAGGG
GGTGCGAGGG GCCTTGCCTG TGAGCCTTAA TAAATGTTA
PKR rs2254958:
TAATGAATTA TTTCTCCTCC TTCAATTTCA GTTTGCTCAT
ACTTTGTGAC TTGCGGTCAC AGTGGCATTC AGCTCCACAC
TTGGTAGAAC CACAGGCACG ACAAGCATAG AAACATCCTA
AACAATCTTC ATCGAGGCAT
C/T
GAGGTCCATC CCAATAAAAA TCAGGAGACC CTGGCTATCA
TAGACCTTAG TCTTCGCTGG TATCACTCGT CTGTCTGAAC
CAGCGGTTGC ATTTTTTTAA GCCTTCTTTT TTCTCTTTTA
CCAGTTTCTG GAGCAAATTC AGTTTGCCTT CCTGGATTTG
TAAATTGTAA TGACCTCAAA
STEAP3 rs865688:
TAGAGATGTCAAACAGGTAGATTCCTCTCCCATTCATATCTCCTATCCTT
GGCCCACAGCCCTTCCCTTCTTGGACTTATCAGAGACCAAGGTGCTGGGC
AGGGCTTCAGGTGGTTAAAAAGTGAAAGTT CTTGAGTGAA
A/G
TCCAAAGGCGCACACCTGAGAGCTGAGTGGGCAAAAGGTCGCTGGCTGAG
TGCTGGGGATAGTCTGGCTTTGGAGTCAGATGGACGAGTCCAAATCTCAG
CTCCTTACCCCGTAACATGAAGCCCTCAGCTCTCTGAACCTCTGTTTATT
TGCAAAACCT TGCCAAGGGC TTCAAACAGG
SLC40A1 rs1439812:
AGTTTGCTATTGAACCAGAAATCAATTACAGTACAGTTCACAAACATTAG
TATACCTTGAGCATCAAGAAAAAGCCAGTTTTCCAATTTGTAAAATGATG
GGAATGGACCATATGATCTCCAAG
G/T
TTTCTTTCATGTCTAATATCCTAAAACCTATGAGATTTCATAAAGTCATA
ATTCGAAAGTCATAAAACAGCAGAGCGATTGGAAAGAGGAGTAGTAACAG
CAAATTCTGGCACTGCATAAACAGTGATGTCAGAAATAAACTTAAATGCC
TAAGTAA
CTLA4 rs231775:
ATTTCAAAGC TTCAGGATCC TGAAAGGTTT TGCTCTACTT
CCTGAAGACC TGAACACCGC TCCCATAAAG CCATGGCTTG
CCTTGGATTT CAGCGGCACA AGGCTCAGCT GAACCTGGCT
A/G
CCAGGACCTG GCCCTGCACT CTCCTGTTTT TTCTTCTCTT
CATCCCTGTC TTCTGCAAAG GTGAGTGAGA CTTTTGGAGC
ATGAAGATGG AGGAGGTGTT TCTCCTACCT GGGTTTCATT
TF rs1049296:
CACCACTGAGTCAGTTCCATCTCCCCAGCGGGGCACCTTGACCAAAGCCA
TCAGCTGAACCACCTTCTTCCTGTCCCTAGGAAAAAACCCTGATCCATGG
GCTAGAATCTGAATGAAAAAGACTATGAGTTGCTGTGCCTTGATGGTACC
AGGAAA
C/T
CTGTGGAGGAGTATGCGAACTGCCACCTGGCCAGAGCCCCGAATCACGCT
GTGGTCACACGGAAAGATAAGGAAGCTTGCGTCCACAAGATATTACGTCA
ACAGCAGGTATGGACCAGCCAGGTCCTCCCACCTTTTCTTCCTAGATGGC
CATAGGC
TF rs8649:
GCCCTGTTATCTCTTAAATAAAAGCTGCTTGCATTGACTCAGGAAAAGCT
GACTTCCTCTTGTCCTTCTGCACAGATGAATTTTTCAGTGAAGGTTGTGC
CCCTGGTCTAAGAAAGACTCCAGTCTCTGTAAGCTGTGTATGGGCTCAGG
CCTAAACCT
C/G
TGTGAACCCAACAACAAAGAGGGATACTACGGCTACACAGGCGCTTTCAG
GTGAGTCTTTTAACCCTGAAACAAATAGAATAATATACAAGCCCTGGCCA
GATTTCTTTTAGGAACTAAGGTAAGATTCTTAGGTTCCTATTCCATTAGT
GCGGCATGTATTAAGAGAGTATATTTCACA
TF rs1130459:
CACAAACACGGGAGGTCAAAGATTGCGCCCAGCCCGCCCAGGCCGGKAAT
GGAATAAAGGGACGCGGGGCGCCGGAGGCTGCACAGAAGCGAGTCCGACT
GTGCTCGCTGCTCAGCGCCGCACCCGGA
A/G
GATGAGGCTCGCCGTGGGAGCCCTGCTGGTCTGCGCCGTCCTGGGTGAGT
GCGGGCACGGGGTAGCACCGCAGAGTCGCTGGCCCGCGCGTTCCCTGCAA
CCCGGGCGGCCACCGCGCAGCCAA
TF rs4481157:
AGTTCATCTTCCCCTATGACTCTGTCCCTAGTCTAAGGTGTCCCACAGGA
AGCTTGAGGGCGGGAAGTTTTCCAGCCCAGGAGCCTGAGCTCAGCGGGGC
AGGAAGAGGGAGCAGCTCCTCCGTGGG
A/G
GACCTTTGAGAGCCCAGGAGCAGGATTTCGAGGGACACCTGGTGGGGAGC
AAAAGGTGCTGAGTCTGTCTTTGACCTTGAGCCCAGCTTGTTTCTCCTGC
ATCCTCCCCCAAAAGGGGCTTTGCCTGTCATTCTGCAGTTCTAGTGTGGG
GTCTGGG
LTF rs1042073:
GGCTGTTAGGTAAAGGTTGCTTGTGTGGACTCAGGTTTGAAGAGCTGACT
CCCCGTGTTCCTTCTCTCCAGATGAATATTTCAGTCAAAGCTGTGCCCCT
GGGTCGACCCGAGATCTAATCTCTGTGCTCTGTGTATTGGCGACGAGCAG
GGTGAGAATAAGTGCGTGCCCAACAGCAA
C/T
GAGAGATACTACGGCTACACTGGGGCTTTCCGGTGAGTCTGTGACTGAGC
TCCATCAGGATGGGGCCTTACCTCATCCCTCAGCATGTCAGCATTGCAGT
TCTAAGGAGCCAGATGTGACCTGTCACAGCAGAGTGGGGGTCATCCTGTG
GGTCAGCTCATGGGTGGCCCCAGTGAGGGC
EGF rs4444903:
AAAGGAGGTG GAGCCTGAAG AGCTTTAAAA AGCAAAGCTG
AGTCATTCCA CTTTTCAAAA AGAGAAACTG TTGGGAGAGG
AATCGTATCT CCATATTTCT TCTTTCAGCC CCAATCCAAG
GGTTGT
A/G
GCTGGAACTT TCCATCAGTT CTTCCTTTCT TTTTCCTCTC
TAAGCCTTTG CCTTGCTCTG TCACAGTGAA GTCAGCCAGA
GCAGGGCTGT TAAACTCTGT GAAATTTGTC ATAAGGGTGT
CAGG
NFKB1 rs4648022:
ACACTCATATGTCAGGCATTGTTCTAGGGACTAGAGATCTCTGCCTTCAA
GGAGCTTATTTTCTAGTGGTATATTTTCTGTTCTGTGTCTTAGCTATCCA
CTTTTTTCATCTGCCTGGACA
C/T
GTGACTTATTCTGTCTCTGGGCCTCTGGTATGAGTGCTCATTTCATTCTG
CCTTATAACTCCTATTTTCTTCCCTACTTTATCTGACCTTCCTACCTTAG
CTTGTTCATTCTTTCCTTCAATCCAGTTGTCATGAAATCTCTTTCTTTCC
TCTACTAATTTTTT
IRF4 rs12203592:
ATGTTTTGTGGAAGTGGAAGATTTTGGAAGTAGTGCCTTATCATGTGAAA
CCACAGGGCAGCTGATCTCTTCAGGCTTTCTTGATGTGAATGACAGCTTT
GTTTCATCCACTTTGGTGGGTAAAAGAAGG
C/T
AAATTCCCCTGTGGTACTTTTGGTGCCAGGTTTAGCCATATGACGAAGCT
TTACATAAAACAGTACAAGTATCTCCATTGTCCTTTATGATCCTCCATGA
GTGTTTTCACTTAGTCTGATGAAGGGTTCACTCCAGTCTTTTCGGATGAT
AAAATGCTTCGGCTGTCAGTCTAATAAGGG
BMP6 rs17557:
GTAGCTACAGGAACAAGTTTCTGTGGAATAAAGAGATGCATGCTTTGATT
TGCATTAAAGGAGTCCACGTCCACCCCCGAGCCGCAGGCCTGGTGGGCAG
AGACGGCCCTTACGACAAGCAGCCCTTCATGGTGGCTTTCTTCAAAGTGA
GTGAGGT
C/G
CACGTGCGCACCACCAGGTCAGCCTCCAGCCGGCGCCGACAACAGAGTCG
TAATCGCTCTACCCAGTCCCAGGACGTGGCGCGGGTCTCCAGTGCTTCAG
GTGGGTTTGTGGGGAGCCTGTGTTTCCAGAAAGCCTTGTTGGCCTCAGTG
AGAACAAAAGTTGTGTCCACAGTCTCAGAT
EDN1 rs5370:
TCAGGTTTTGTTTGTGCCAGATTCTAATTTTACATGTTTCTTTTGCCAAA
GGGTGATTTTTTTAAAATAACATTTGTTTTCTCTTATCTTGCTTTATTAG
GTCGGAGACCATGAGAAACAGCGTCAAATCATCTTTTCATGATCCCAAGC
TGAAAGGCAA
G/T
CCCTCCAGAGAGCGTTATGTGACCCACAACCGAGCACATTGGTGACAGAC
CTTCGGGGCCTGTCTGAAGCCATAGCCTCCACGGAGAGCCCTGTGGCCGA
CTCTGCACTCTCCACCCTGGCTGGGATCAGAGCAGGAGCATCCTCTGCTG
GTTCCTG
HFE rs807212:
AAGAGCCAATTTCAGTGCTACCATGTTTGTATAGCAGTATTTATGTCTGT
CATCCTCAGTCATTTTACTTCACTTGTTCTTAGCCAAACGGCCGAGAAGC
GATGGTCATTTTACTTCAAAAATGAAAAGAATTAATATTTTTACGTTTCC
CTTAAAGACCCTATGTTTAACCTCCACTCC
C/T
GGGTAAAATGGTCTAGTCCCTCCTTTTCATATCATCTCTGATATCTTTTG
CACAGCCACTATTACCTACCGTTTTCTAGATCCCTATTCTTCAAACACCA
CCATGAAGGTAGAGCCTGTCTGAATTATTTTCTTGTCCCCTGAACTCAGT
ACATTGTTAG
HFE rs1800562:
TGAAGTGCTGAAGGATAAGCAGCCAATGGATGCCAAGGAGTTCGAACCTA
AAGACGTATTGCCCAATGGGGATGGGACCTACCAGGGCTGGATAACCTTG
GCTGTACCCCCTGGGGAAGAGCAGAGATATACGT
A/G
CCAGGTGGAGCACCCAGGCCTGGATCAGCCCCTCATTGTGATCTGGGGTA
TGTGACTGATGAGAGCCAGGAGCTGAGAAAATCTATTGGGGGTTGAGAGG
AGTGCCTGAGGAGGTAATTATGGCAGTGAGATGAGGATCTGCTCTTTGTT
AGGGGGTGGGCTGAGGGTGGCAATCAAAGG
HFE rs17596719:
TTAATAAATGTATATTGTATTGTATACTGCATGATTTTATTGAAGTTCTT
GTTCATCTTGTGTATATACTTAATCGCTTTGTCATTTTGGAGACATTTAT
TTTGCTTCTAATTTCTTTACATTTTGTCTTACGGAATATTTTCATTCAAC
TGTGGTAGCC
A/G
AATTAATCGTGTTTCTTCACTCTAGGGACATTGTCGTCTAAGTTGTAAGA
CATTGGTTATTTTACCAGCAAACCATTCTGAAAGCATATGACAAATTATT
TCTCTCTTAATATCTTACTATACTGAAAGCAGACTGCTATAAGGCTTCAC
TTACTCTTCTACCTCATAAGGAATATGTTA
HIST1H1T rs198844:
GTGACACTGAAAGGGCCTCGGTGATCAACTTGGACACAGAGAGGTTCGGC
ACTTTGCGACTTGCACTTATCAAGCCAGCCGGCTTCCTCCCTCGCTTCTG
GTTGGAAGTTTCTCCATAGCGGCTA
C/G
ACCAGCACTGGCAGAAGCTGCAGGCACGGTTTCAGACATAACAACAGAGA
AACGCAAGATGTAATAACCAGCGAAAAGCATGAAACACCCGGGCGGCCTC
GGGGCCTTATATAGGGTAGGGCGCGCTGTGATTGGTGCATCACCTAGGCA
CCGC
UBD rs2534790:
GGGACAAATTATCTTATTTGTGTTGTAACTTGGTAATTCCAAAAAAGAAG
TTCCAAGAAAGAGAGGGACACTGGCTACTGAATAGGAGCTAGAGGACCAG
ATAGATAGTGGAAGAGGGGGAGCCATTGTGGTGGGGAGTAGAAGTGTAAA
GGAGG
A/C
AGGCATCCTAGGTAACTGTCTTGTGGCTTTCACTTCCCAGGTGCATGTCC
GTTCCGAGGAATGGGATTTAATGACCTTTGATGCCAACCCATATGACAGC
GTGAAAAAAATCAAAGAACATGTCCGGTCTAAGACCAAGGTTCCTGTGCA
GGACCAGGTTCTTTTGCTGGGCTCCAAGAT
HLA-G rs1736939:
GTCTTCCTAAACCTGTGTTTTCATTTTGAATCCTCCTTCAGGCTTATACA
GAGGTGGCAGAATGCAGTTTCTGGCAGTTGTAAGACTGAGGTCCCTGTTC
CTCACTGGCTGTCACTGTAGGAACAGGGAGGGCTGCACT
C/T
AATGCATGGTGCCCACCAGCGTCCTTTCCTACACAGCCCCTTCATTTTCA
AAGCCCACAGTGGAGGAAACCCCTTATGCTGAATCCCTCTCACACTGTGA
ATCTCTATGCTCAGGAAGAACCCAGTCCTTTCAAGGACTCTCCTTATTAG
GACAGTCCA
HLA-G (indel) rs1704:
CAGGGGACATAGCTGTGCTATGAGGTTTCTTTGACTTCAATGTATTGAGC
ATGTGATGGGCTGTTTAAAGTGTCACCCCTCACTGTGACTGATATGAATT
TGTTCATAATATTTTTCTGTAGTGTGAAACAGCTGCCCTGTGTGGGACTG
AGTGGCAAG
C/T
TCCCTTTGTGACTTCAAGAACCCTGACTCCTCTTTGTGCAGAGACCAGCC
CACCCCTGTGCCCACCATGACCCTCTTCCTCATGCTGAACTGCATTCCTT
CCCCAATCACCTTTCCTGTTCCAGAAAAGGGGCTGGGATGTCTCCGTCTC
TGTCTCAAA
ZNRD1 rs9261269:
GGGGATATGACCAGGCCTCCCTAACCCACCAGTTTCTTCCCAGGTTGACA
GGCGCTGCCCTCGATGTGGTCATGAAGGAATGGCATACCACACCAGACAG
ATGCGTTCAGCCGATGAAGGGCAAACTGTCTTCTACACCTGTACCAACTG
CAAGTG
A/G
GTATTCTTTCCCCTCCCTCTGCTCAGTCTGTTTGCTAACTAAACAAATCC
AGTGATTTATTTTTTTGTACGAAATGGCCGTTTCCCTTGGTCCCATCCCT
TATTTCTGTGCAGTTCTGGTAATAGGGAGATTTGTAGTTGTTTTTTATTT
TTTTAAGTTACACTTTTTTAAACCTTTTTA
HLA-E rs1264456:
CACAGGAAGA AATGGCAAAG TAAAAATTCA CACCCAGGAC
TCCCTGGGCT TTCTCACCGC ACATGTTGCC TTCTTACTGG
ATATCACCTG ACAGAATGAG ACTCAGGTGA TTACAGGGAT
TCACCAGGAA AACGGGAAAG TCGGCATGAC CAGAACTAGA
ACA
C/T
GGGCCAGTGA ATGCAGTTCT GGGTGGACCA TGGCATTGGA
AGCCAAAGGA TAGCTTGAAT GTGGTTAAAA AATTAAAACA
ACAAGGCACA AAACGCACAA ATGAAATACA AATGATGCTC
AAACACAGCT TTTATTTTAC TTCAAAGTTT ACCTCAGATC
AGCCTGGGAA
DDR1 rs1264328:
GGAGCCGAGA TTCCCAGGGG CCTGAGAGGG AAATCCCAGC
CATCCTGGGGCCCAGAGAGC AGCACCAAAG ACCAAGAGGG
CCTGATTACC CATCCGTGGT CCCCAGAGCC CATTCCACAT
CTCCTGCATC ACTCCGAACC CCAGAGGCCC CCTGTGTCCC
C/T
GAGAACCCCCAAATGACCCT CTACCATCCC CTCCCATCCT
GGGCTTCCCT CCCCTTCAAG CCAGTGGCAGCCTGCTGCCCA
GGAAGGAGAGGATGGGAAACAGCTGAAAAAATGTGAGGAG
AGGCACGTA GGAGAGGGGA GAAGGCAGCT TCAGGCCTGC
AGACCACCTG GCCACAGGAG
DDR1 rs1264323:
AAAGGTTTAATAACTACAATAAACTAAGGCCCCTTAGGAACTGACAAGAA
AAAATATATAAGTAACCCAATAAAGAAACAGCCAAAGAATGTGAATAGTC
ATTTCACAGA AAAGCAAAG CCAAATTGCCA ACAAACATTT TTTTTA
ATGGTCAAATTCAC
C/T
AGCCTCAGGGAAATACCAAGAGACTGACAAGATATTTTTTTAAGGCTGT
AACATATAAAAGTGGTAGAAAGTGATGAAAAGGGAGGCACCATGCTCAAT
GGCAGAAATGAGTGTTATCTTCTATTTGGA AAGCAATCTA
GCAATGTCTA TTATAATTAA AAATGCACAT CCTCTTCGAC
DDR1 rs1049623:
TTCTCTCTAG ATGGCTCCCC ACTCTTCACG GCCTCCCCTC
CCTTCTTCCA GATGCCATCC CTGGTCCTCA CCTGGCATTC
TTGGTGGCAT CTGGCCGTAA GATCTTGACA GCTACCAGCA
AAGGGTGTCC CTTACGCACA TTAAGGGGGA AATCAAGACT
A/G
ACCAGATCTT GAGGGCTGTC GACCTCACAC AGGTGCACCT
GGAGAAAGAA GTTCGTTTGC TAGGCGGTCA CAGGGTCAAA
CGGATTAACA CGGTTACAAA TGACTAAGGT TCCTGGCTAG
GGGGATGCGC AGGCATGGCA TCAGAGCACA CAATAGGGCC
AGACACTGGGTAGGCACCCT
HLA-C rs9264942:
TCCACATGTG CACAGACAGA CACACACACA TTACACAGTC
CCAATTCCTT GATTCAGTTT GGGCCCTGGG TAATTCCAGT
TCAATCTCTT TTAAGAAATT TAAGAATCTG AAAGAGAAAG
ACCTGAGAAT TTTTGTCCCA CAAGAGACAG ACCCACTTCC
C/T
AGGCACTGTG GGACTTTCTG AGCCCCATGT GGCCCTGCTC
CTGGAAGCTC ATGGAGGAGC GGGAAAATCT GACTTAACAT
CAAGGTTCTG AAGTCCAGAG GCAGCCCTAG GAACTGGCCT
TCCCTGGGTA CCAGGCCTCC GGGAGTCCAG CAGGTCCCCT
TCCTCCTATC TCACCTATGA
MICA rs1051792:
GGAATGGAGA AGTCACTGCT GGGTGGGGGC AGGCTTGCAT
TCCCTCCAGG AGATTAGGGT CTGTGAGATC CATGAAGACA
ACAGCACCAG GAGCTCCCAG CATTTCTACT ACGATGGGGA
GCTCTTCCTC TCCCAAAACC TGGAGACTGA GGAATGGACA
A/G
TGCCCCAGTC CTCCAGAGCT CAGACCTTGG CCATGAACGT
CAGGAATTTC TTGAAGGAAG ATGCCATGAA GACCAAGACA
CACTATCACG CTATGCATGC AGACTGCCTG CAGGAACTAC
GGCGATATCT AGAATCCGGC GTAGTCCTGA GGAGAACAGG
TACCGACGCT GGCCAGGGGC
BAT3 rs2077102:
CTTCGGTCTG TCTCTTCTGC CACCCACAGG ACAGCAGGTG
CCAGGCTTCC CAACAGCTCC AACCCGGGTG GTGATTGCCC
GGCCCACTCC TCCACAGGCT CGGCCTTCCC ATCCTGGAGG
GCCCCCAGTC TCTGGGACAC TGGTGAGCAA GGGTCGGGGA
G/T
TTCTAGTGCG TAACAGTCTA GGGAGAGACT CCTGTGGTGG
TGCATGGAAG GGCAGGTCTG AAATTCTCCC TTGCTCTCTA
TCCAGCAGGG CGCCGGTCTG GGTACCAATG CCTCGTTGGC
CCAGATGGTG AGCGGCCTTG TGGGGCAGCT TCTTATGCAG
CCAGTCCTTG TGGGTGAGTT
HSPA1B rs1061581:
CCAGGGCGAG GTTCGAGGAG CTGTGCTCCG ACCTGTTCCG
AAGCACCCTG GAGCCCGTGG AGAAGGCTCT GCGCGACGCC
AAGCTGGACA AGGCCCAGAT TCACGACCTG GTCCTGGTCG
GGGGCTCCAC CCGCATCCCC AAGGTGCAGA AGCTGCTGCA
A/G
GACTTCTTCA ACGGGCGCGA CCTGAACAAG AGCATCAACC
CCGACGAGGC TGTGGCCTAC GGGGCGGCGG TGCAGGCGGC
CATCCTGATG GGGGACAAGT CCGAGAACGTGCAGGACCTG
CTGCTGCTGG ACGTGGCTCC CCTGTCGCTG GGGCTGGAGA
CGGCCGGAGG CGTGATGACT
SKIV2L rs419788:
CAACAAGGTC AACCTTGTCA TGTCCATCTC TGTTCCTTAG
GAGAAGGACA TGACTTCTCC TACACCCCAC TCAAAAACTA
AAACTAACCT TTTGGTGCAA AGTCCATGCC TTTCTTGAAA
CCAGGTGGAA TAGTAAGAAG ATCTGTAGGA TAGGGACATG
A/G
AATCAGGTCA CTGCACACTG GTGAACAAAT TGTGTACATT
ATATAAACCT AAAAGATACC ATTTACAGGA CAGATGCTGT
AGATAGGGAT GTTTGCTATG ACACTTTCCC AACAGATGAC
AGTAAAGGTT GTTGTAGAAA TTTCCCAGCA GATGACAGTA
AAGGTTGTTA TGGACAGAAT
NOTCH4 rs3096702:
GGAAGTGAAA ACTACCCAAA TTCAGTGTTT GTTACAGACA
ATTCAGACTG CAAAATTTAG GGTAGACTAT GTTCATTTAT
CACTGATAAT GACAGTCTTA ACATTCCCCT ACAACAGGAA
GACCAAGATT TCCCCAAAAC CGGCCAGCAT CTTGCCCATT
C/T
GCCAGAAGGAGAAAAATAAG TCCTGGCAAG AGCCAAGATA
AGGCCCAGAAGCCCCTGGGT TCCTTTAGCC AAGGTGAGTG
GTTTCAAATT ATGACAAGTT GCAGGTTCTC TGAGAAGCAT
CTGTAATAAC CTGGCAAATT AAGCATCCTC TCCTGGGAGG
AGGAATACAG AACTCTGTAA
BTNL2 rs9268480:
AGGATTTGAT ATAAATTTGA TGATGAATAA GCATTAAGAA
AATTTCAAAT GTCAGAGAAA TTGTCCAGGA ACTAGCATAT
TAAAGTGGCA GGAGCAGGTA TTGAATACAA AATATCTATC
TAGAATTCTT ACTTACCACC TTCAGATCCA AACTGGCCTC
C/T
TGGTAGACAT CATCTTTTTC AAAAAGGCAG CGGTACTGCC
CGTCGTCCGA AGGTCTGGCA CTGAGTATCT GCAGGGTCAG
TCTGCCCTCG TCAATGGCGT CACTCACCAG TACAGTCCTC
CCTCTGTACT CTGCCATCTG CTCTCCAGCC ACATGGTCCC
CATCCATATA CACATGCACA
HLA-DRA rs7192:
CTTCTTCCCA CACTCATTAC CATGTACTCT GCCTTATTTC
CCCCCAGAGT TTGATGCTCC AAGCCCTCTC CCAGAGACTA
CAGAGAACGT GGTGTGTGCC CTGGGCCTGA CTGTGGGTCT
GGTGGGCATC ATTATTGGGA CCATCTTCAT CATCAAGGGA
G/T
TGCGCAAAAG CAATGCAGCA GAACGCAGGG GGCCTCTGTA
AGGCACATGGAGGTGAGTTAGGTGTGGTCAGAGGAAGACGTAT
ATGGAGA TATCTGAGGG AGGAAAACAGGGTGGGGAAAGGAA
ATGTAA TGCATTTAAG AGACAAGGTA GGAACAGATG
TGGCTCTTGA TTTCTCTTTG
HLA-DRA rs3135388:
TGCAATGTTT ATGGATTCTT CTGTCTICCT TCTCCCCACT
CTAACCCCAT CTGCTCCCCT CCATCCCATG CATTCTGAGA
TCCATACCTT GGGGTTTCAG ATTCACTCTA CTGAAGATAG
AGTTATATCA TTGCTCAGTA GAGATCTCCC AACAAACCAA
C/T
CCCACTTTAG GTTTTCCTGA TGAGGACTAG ACCACAACAA
GAGGGTTGCC TGCAGATGCA CAAAATGAGA CCAAGCCCAA
ATGAACCGGG ATATGTCTGA TGAATTCTAG AATTTATAAG
ATAAATTCAA CATTCAGATA TTTTACCGGG AAAGGATCAC
ATATATTCCC CAGGACCGAC
HLA-DQA1 rs1142316:
TAACATCGAT CTAAAATCTC CATGGAAGCA ATAAATTCCC
TTTAAGAGAT
A/C
TATGTCAAAT TTTTCCATCT TTCATCCAGG GCTGACTGAA
ACCGTGOCTA
HLA-DRB1 - DQA1 region rs2395225:
CCCTGGTTAA TGTAGTCATC ACTGTTCAAG CCCAGTCTCT
TTCAGATGTT GAGACAGTGG CCCTAACTCT GTGTGGCTGG
CCCAGAGCTG TGCACCTACC CTCACTTTCA TACCACATTA
AYITCAGATC CTTATTGTCA
C/T
GGGTTTCCCA ACTACTTTTT TTTCTTCAGG GGAAACCTCC
ACAATGTAGT TTCTAATATG TTGAATTCAT ACTCCAGAAA
GTGTCCTGTA GAATAATGTC TTACTGAAAA CGGCCATCAC
AGCCAGGAGT CCTTAACTAT GTTCTTCGAT ACCCTTAGTT
ACAGTTTGTT GTCATGTTCT
HLA-DRB1 - DQA1 region rs9271586:
CATCACAGCC AGGAGTCCTT AACTATGTTC TTTGATACCC
TTAGTTACAG TTTGTTGTCA TGTTCTTCAC ATCTTGTGTG
AAGATTGTTC AAGTATTGGC CAAAGGATAT GTCACTATCT
AAAATTCACA TTGAGAACCT CAGAGTAACT AATAATAAGT
G/T
TGATGCTTGT AGGAAAAGAA GAGCTGTTTG GTCACAGGAT
GTGGAAATTA GAATAGGGTT GTGGTTGAAG GGGAAGGATG
ATGACATAAA TCTTTGCATA AACCACATTA ACATGAAACC
TTGATATTAT CATTACATAC TTTTCTTTTT ATCTAATAAG
GCAAAGTAGA GAAGTCAGCA
RXRB rs6531:
AGTGGCCTTA CCTTGCGTAC CCAGGGAGCC AAACTTGCTG
ACCTCGCCAC CTCTTTTCTC CTTCTCTTCC ACTGATGTGC
TTTGAATCCC TTGGCCTGAT TTCTGGCTCC TGACCCTTGC
TGCCCCACCC AGGCTGGAAT GAACTCCTCA TTGCCTCCTT
C/T
TCACACCGAT CCATTGATGT TCGAGATGGC ATCCTCCTTG
CCACAGGTCT TCACGTGCAC CGCAACTCAG CCCATTCAGC
AGGAGTAGGA GCCATCTTTG ATCGGTCAGT GGCCCTCGGC
TAGGCTGGCA TGTAGATAGA GGGGGTGGGG CTATAGGCTG
GTCCGTGTCC AAGGC
RXRB rs2076310:
AGATGTGAAG CCACCAGTCT TAGGGGTCCG GGGCCTGCAC
TGTCCACCCC CTCCAGGTGG CCCTGGGGCT GGCAAACGGC
TATGTGCAAT CTGCGGGGAC AGAAGCTCAG GTATGTGGCT
CAGAGGATGA ACAGAGAGGG AGAGTCTGGG CCATGTATCA
C/T
CACCTGTGGG ATTCCCAGGG CTTATGGAGT TTGGTCAGAG
CAAGTGACCT GGGGGAGGCC TGATGGGAGT AAAGAAGCTG
AAGCTGAGAT GTAGGACGCG ATTGGGGGGA AGGTCAGAGG
GAAAAGGAAG CAGCGTGTAG GGTTTCTGAA CAGTGAGGAG
ACTGGGACTG GATCATCACT
HSD17B8/RXRB rs365339:
AGCAGAAACT CATCCTGGGT GATGCCCGCA CAGGACACAA
CGACAGATGG GGGCGAGAA AAGCAGGCCT ATGGGAGGGG
GAGGTTACGC ATCAAAAACC CCCCACAAAA AGCCGGGGCA
GTGGAGGCAA TATCAGAGCT TTAGAGGGGG AAAGTGGCCT
A/G
GCGTTCACCT GCACTTGTTC CAGCAGGCAC CTGGCGGCCC
TGGCCTCAGA CACGTCAGCC TGGAAGGCAG CATGGTTCCC
TCGGGGCGGC CCCTCCTTGC TCCCTGGCCC GCCCAGCAGC
CGCACCGTCT CCTGTGCCGC TGCCCGGTCC AGGTCGCAGG
CAGCTACGGT GGCCCCCTCT
HSD17B8IRXRB rs421446:
GAGGGCCACC TGTTCCAAGA CCCCCTTTCA AGGCCAGACT
GGACACCAAG ATGGGGCCAT GAACAAATCA CCCTTGGGGA
CCATAAGAAC CCAGGGAGTT GGGGGGAGGG GACTGGTGCT
GCAGAACCAG TGGAAAGGGG TGACGCACGA ACCCCTCCCT
C/T
CAAAAAGACC CGGAGTGTCA CGCATACACA GTGACACATA
CTCTTTCCTC TCACACCCGG CGGCGGGGGT TGCCCTGGGA
GACCAGGCAG AGAAAGGGAA CAATCCTTCG GGAAAGGGAA
AGGAGGGGGA GGTGGGGAAG GGTCTGAGGG CTTGGACACA
AGAAGAGCCG GAGGTGGCAG
DAXX rs2239839:
AGGGCGAGAG AAAAAAGAGA AGAGCTCGGC TCCAAGGCAC
CTCTTCCCAC TCTGCAGACA CCCCCGAAGC CTCCTTGGAT
TCTGGTGAGG TGTGGATGGG GTACAGCCTT CAGAGAGACA
TTGTCCTTCC CCTGCACTGG CCACCAGGGA GTCCAGGTTG
ACTGATGGGG
G/T
AGCATGAGAA GGAAAGCAAG AACCAAACCC TCTGGGGCAA
GGGATTCCTT AGAGAAACTT CTTTGTCTCC CAGGGCCCTA
GTGGAATGGC ATCCCAGGGG TGCCCTTCTG CCTCCAGAGC
TGAGACAGAT GACGAAGACG ATGAGGAGAG TGATGAGGAA
GAGGAGGAGG AGGAGGAAGA AGAAGAGGAG GAGGCCACAG
ATTCTGAAGA
DAXX rs1059231:
GGGTTTTTAC TCTTCTAGTC CCTTCAAGGG CTGAGTGCTC
TGACTTTATG TCTTCCCACG TAGGCGTTGA CCCTGCACTA
TCAGATCCTG TGTTGGCCCG GCGCCTTCGG GAAAACCGGA
GTTTGGCCAT GAGTCGGCTG GATGAGGTCA TCTCCAAATA
T/C
GCAATGTTGC AAGACAAAAG TGAGGAGGGC GAGAGAAAAA
AGAGAAGAGC TCGGCTCCAA GGCACCTCTT CCCACTCTGC
AGACACCCCC GAAGCCTCCT TGGATTCTGG TGAGGTGTGG
ATGGGGTACA GCCTTCAGAG AGACATTGTC CTTCCCCTGC
ACTGGCCACC
DAXX rs2073524:
ATCAAAAGTC CCCCCGCACC GCGCTACGCT CTCGCGATTC
CTCTTAGATC CCAACCGTGG GTCCGGCCGG TCCGCTAGAT
GCGCTTCCCG CCAAATCCCC CTCCCCCAGT TCAGCCCCCG
GCCGCTCCAC TCCCTTTCAG GGACAGGAAG GTACCACAGC
T/A
TTCCCCTCAG ACTCAGCGCC CAGCTCTCCC CAATACCTCT
CCCTCTATAT CCCCGCCCCC GCCTCTGATC CCCGCACCGT
CCGGCCCCCA CCTCAGAAAC CGTCTCTCGA GGCGACCCTC
VEGFA rs1570360:
CCCTTCATTG CGGCGGGCTG CGGGCCAGGC TTCACTGAGC
GTCCGCAGAG CCCGGGCCCG AGCCGCGTGT GGA
A/G
GGGCTGAGGC TCGCCTGTCC CCGCCCCCCG GGGCGGGCCG
GGGGCGGGGTCCCGGCGGGG CGGAGCCATG CGCCCCCCCC
TTTTTTTTTT AAAAGTCGGC TGGTAGCGGGGAGGATCGCGG
AGGCTTGGG GCAGCCGGGT AGCTCGGAGG TCGTGGCGCT
GGGGGCTAGC ACCAGCGCTC
IL6 rs1800797:
GAGAGCAAAGTCCTCACTGGGAGGATTCCCAAGGGGTCACTTGGGAGAGG
GCAGGGCAGCAGCCAACCTCCTCTAAGTGGGCTGAAGCAGGTGAAGAAAG
TGGCAGAAGCCACGCGGTGGCAAAAA GGAG TCACACACTCCACCTGGA
GACGCCTTGAAGTAACTGCACG AAATTTGAGG
A/G
TGGCCAGGCAGTTCTACAACAGCCGCTCACAGGGAGAGCCAGAACACAGA
AGAACTCAGATGACTGGTAGTATTACCTTCTTCATAATCCCAGGCTTGGG
GGGCTGCGATGGAGTCAGAGGAAACTCAGT TCAGAACATC
TTTGGTTTTT ACAAATACAA ATTAACTGGAACGCTAAATT
TFR2 rs10247962:
ACCCAGCTGA TTTTCAGATG CTCACATCTT TTTAAGGCCT
CCATCATTCA CTCACAGAGC TCATCTGTGC CCCTGATGTC
AACCAGGACC TCTGTGGGGA CAGATGCCAA ATCTCCCCAC
CCA
A/G
TGACCCACTG GAATCCTGCC CTCCAGCCAT CTGGACCTCC
CCACTGGGTT TGGGAGCACC TGGACATATC AGTACCGATC
TCTTCCCAAA CCTGGGCGTT GGGCCCACAC TCATGTGGCC
CATGGCTTTC TGCAGGTGTC AAGCTGTCAC CCTCAAAGGG
GAGTGAGCAT GGGGTGAGCA
SLC39A14 rs11136002:
AGACATCGCC AAAGATGCAC AGATGGTAAA TAAACGTATG
AAAAGATGCT CCACATTATA TCTCCTTAGG GAACCACAAA
TTAAAACAAG GCACCCATTC CATACCTGGT AGAATAGCCA
AAATCCACAA CACTTAACCA
C/T
GCCATATGCT GGTGAGGTTG CAGAGCTGCA GGAACTGGTA
CAGCCACTTG AGAAGAGAGT TCTTAATAAA ATTAAACAGG
ATTACAAAAC CACATACATA ATCTTATCAT ATGGAGCAGC
AGTCATACTC CTTGGTGTTT ACCCAAAGGG GATGAAAACT
CATGTCCACA CAAAAGCCTG
SLC39A4 rs2272662:
GGCTGAGTCT GGAAGAAAAG CTCTCACAGC CGCCTCACCC
GCCCCCAGGG TATCTGTACG GCTCCCTGGC CACGCTGCTC
ATCTGCCTCT GCGCGGTCTT TGGCCTCCTG CTGCTGACCT
GCACTGGCTG CAGGGGGGTC
A/G
CCCACTACAT CCTGCAGACC TTCCTGAGCC TGGCAGTGGG
TGCAGTCACT GGGGACGCTG TCCTGCATCT GACGCCCAAG
GTCTGCCCCC ACAAACCCGC GACCCTGGCC CTCCGTTCCC
CACCATGGAC TCCCAGGCCG TGCCCTCCCA GGGACCTTAC
CCACCCCACC TCCTGACCCC
LCN2 rs878400:
CATGGAGAGG CCCAGGTCTC ATCCATGCAT GAAGOCAGCA
AGATGCTTCC TGGCGGTCCT TACATCTCAG GAATCCAGTC
TGACTCCCCA TTCTGGTTTC CGGATCTTGT GAGTAGTGTT
CAGCGTGGCC ATGAATGGTT AACCCTCTGA
C/T
GTGTTTGAAG GCTGGGCAGG AGGTGACTGG CTAGGCTTCT
AGGAGCCAGG TACCACACCT GGAAGGAGTC TACAGTCAAG
ATGCCCCCAG GAGGCCCAGT CACAGATGCA GGAAGTCTTG
KLRK1 rs1049174:
AAGAAGAGAG ATCCTAAAGG CAATTCAGAT ATCCCCAAGG
CTGCCTCTCC CACCACAAGC CCAGAGTGGA TGGGCTGGGG
GAGGGGTGCT GTTTTAATTT CTAAAGGTAG GACCAACACC
CAGGGGATCA GTGAAGGAAG AGAAGOCCAG CAGATCA
C/G
TGAGAGTGCA ACCCCACCCT CCACAGGAAA TTGCCTCATG
GGCAGGGCCA CAGCAGAGAG ACACAGCATG GGCAGTGCCT
TCCCTGCCTG TGGGGGTCAT GCTGCCACTT TTAATGGGTC
CTCCACCCAA CGGGGTCAGG GAGGTGGTGC TGCCCCAGTG
GGCCATGATT
KLRK1 rs2617160:
ATCTATGCCC ACACCACCAT GATGCATCCA GTCTCGTCTG
GACACGCATG GGCATATTGA AGCAGAAGTG AAATGATGAC
TAATGTAAAA GTAAAAAAGT CTGCAAACAT ATTTTAAGAA
ATATGTATAT ATATATTTTC AGAACCTATT TTCCATTCAG
CTAGGTATTA
A/T
GTACTGGGCT ACACATACTG ACATATAATG TTAACTGGTG
TATTGTAATT ATATGAACTC AAGGCAGAGA TTCCATAAAT
CTGGAATTTA TACTTTGGGG AAAAACAGGT CATCATCTTG
GCAATTAATT AATTTTCTCT GGCACAGCTT CCTAAGCCAG
GAATGATTAA ATGATTTTTT
KLRC4 rs2734565:
CCAATAATAA GTAGAAATGC TCAGTTAAAA TCATTATACC
CTCTTGTTGC ATTTAATTAA CTGAAATTTC CTACTACTAT
AAGATGATAA GAGATAAATA ATTTTACTAT ACTTAAAAAG
CAGTTTTGTT CAGTGATGTT TAAGATGTGT AGGGTGGATT
TTTGTTGGCG GGCTTGTTTT
A/G
TATGGGAACA CAATTAAGGG ATGAGAGGTG GACCTTTTAT
TGTGCATGTG CGTATGAGTG ACTCGTTATT TTAAAATATA
TATTTAACAA CTTATGAGGA TGCAGATATT GTGTACCTGT
ATGTTTATAG CTTTGCAAAT ATATAAAATA ATTTTCATTT
GTAAACATAT TGTTTTGCAT
KLRC4 rs2617170:
AGGACATGCC CTCATATAAT CTTTATTTTA TAAACATTTA
TGGCTCAATG TTATAGTTTA TTATCCCAAA ACATTTTATT
ATCATTTTGC ATCCCTTTAG AGACAAAATA TAAACTGTAC
TAACATCAGA ACATTGACAA TCATAATGTA CCTTTCTGCA
TTCTTCTATT CAGGGAAAAA
C/T
TGTTCTGCTC CAGTACTCCA ATACCTAGAA AAATTAAAGT
GATTCTTACA AAATTAATAT CTAGACAAAT TATAATAAAT
TCAGTTGCTT ACTTTGAAAT ACAAAATTTA AAATTATTTT
AAATTGGAAC AATCTGAAAT AAAAATGACT TTTCTATAAA
AATAATGAGA TCTTTAAAAC
KLRC4 rs2617171:
AAAATGACTT TTCTATAAAA ATAATGAGAT CTTTAAAACA
AATATTTTTA AAGCCATTAG CATAAAACTT CACCATCTCT
TATAGTATTT GATCTAACCA CTTTCAAAAA TTAATTTGTT
TTTCTAAATA TTTTTTCTCT TAAAACATGT CTTTGAGTCA
TGAAATCAGA ATACATCTCT
C/G
TGTGTGTGTA TCATATATAC ATATATATTT AGTACACACA
AAAAAATAAA TGTTTTCTAC AATTATTCTG TTATTTATAA
ATTTGAAAAG TTCAGAAGCA GCATATTATC TTGGGGTTCA
GAGATATACA TTAAACAGAG AATTCTAATC CTCATTATTA
TGAAATGTTT CAAGGCGCTT
KLRC4 rs1841958:
CATTCAACTG CACATCCTAG AACAATAATA TTGAAGATCT
ATTTAATGTT TTACCTTTGC AGTGATATGT CTTGTCATTC
CCTTGATGAT CCGAAGAAGC ATTTTGAAGG TTTAATTCTA
CTTGGAATAT TTCCTGTTTG GTTCCTGAAA TGGAG
A/C
TTTTATTGCC CTTAAGTTTC CTTTGCTGCC TCTTTGGGTC
CTGGGCCAGA CTCACTTCTG AGTAGGTTCC TCTTTGTTTA
TTCATCTCTG GAG
KLRC1 rs1983526:
CTTCTCCTGT TAGTGTCCTG GGCTGATGAG ATTGCCTTCA
GTATCATGGT TGAATGGGGT TACAGCCAGA TCACATGGTT
GCTTACGGGT CCAGAGTGGG ATCTTTGGTT CATGATCCTT
TTACTAGGGC TTCTAGCAGG
C/G
TTGTATCCTA TTTAGTGCCT CAGAGGGCCA AACTGGCTCT
AGAATCATAC TGTATAGGGC TGGGGAGGGG ATGAGGGTCC
ACTTCAAGGT CTGTAAATGG TGGGTCTATT ATTAGGTGTG
TAGTTGGGGA AGAGTTTATC TAGTTTGCTG GGAAGGCTGC
TCATGGGTCT CTGAGTGGGT
IFNG rs2069727:
TGTGGTATTT CTTTCCACTA GCATTTTGTT GGCTTTCGCT
TTTCCAGTTA GCAGCTCTTT GAATTATCTT TCTAAGATAC
AGATTTAATT ATGTCACTAT TCAATTCAGA GGTTCTGCTA
TGGAATGTAG TTTAAACTGC TTAGCTTGGC ACACAGAGAT
TTATTTCTAG CCCCTTCTCC
A/G
TTTCAGAATC TTCCTCTCCC TCATCCAATG CTGGCAAACA
CCAGTGGGGG TGGAGTAGTG GGTGTAAGCT CTAGGGAGAA
GGCTTGGATT GGAATCCAAG TTATTCCATT ACAAGTAGTG
TGACCTTTAA TACATTATGT ATATTGTCTA AGTTTCAGCT
TTATTGTCTG AAAAAGAAAA
IFNG rs2069727:
GAATTATCTT TCTAAGATAC AGATTTAATT ATGTCACTAT
TCAATTCAGA GGTTCTGCTA TGGAATGTAG TTTAAACTGC
TTAGCTTGGC ACACAGAGAT TTATTTCTAG CCCCTTCTCC
ACCTTCCTAT TTCCTCCTTC
A/G
TTTCAGAATC TTCCTCTCCC TCATCCAATG CTGGCAAACA
CCAGTGGGGG TGGAGTAGTG GGTGTAAGCT CTAGGGAGAA
GGCTTGGATT GGAATCCAAG TTATTCCATT ACAAGTAGTG
TGACCTTTAA TACATTATGT ATATTGTCTA AGTTTCAGCT
TTATTGTCTG AAAAAGAAAA
TP53 rs1042522:
TGAGGACCTG GTCCTCTGAC TGCTCTTTTC ACCCATCTAC
AGTCCCCCTT GCCGTCCCAA GCAATGGATG ATTTGATGCT
GTCCCCGGAC GATATTGAAC AATGGTTCAC TGAAGACCCA
GGTCCAGATG AAGCTCCCAG AATGCCAGAG GCTGCTCCCC
C/G
CGTGGCCCCT GCACCAGCAG CTCCTACACC GOCGGCCCCT
GCACCAGCCC CCTCCTGGCC CCTGTCATCT TCTGTCCCTT
CCCAGAAAAC CTACCAGGGC AGCTACGGTT TCCGTCTGGG
CTTCTTGCAT TCTGGGACAG CCAAGTCTGT GACTTGCACG
GTCAGTTGCC CTGAGGGGCT
LIF rs929271:
GCTATTTCAG AGGCAGCATG GGGACACAGA AACAAGGACA
GGGTGGGCCA CAAGGACTGT CTTGCCCACT GCTCCAGGGG
GCACAATATC TGCCAGGAAC AGTGCGCCTC ACAACACAAT
GCTGGGGCGC CCAAGAACAG TGTGAACCAG CCCCCTGGAA
G/T
CAAGACAGAA AGGCACCCGG CCTCTCCACA AATTGGCCCA
GCCCCTGCAG CCTGGACCCT GACACCCTAA AGCAAGTCAC
AGTAGGGGAT GGGGGGGGGT GGAGCAAGGC CCCCCACTCC
CACTCAGGCC TCCCCATTCT CTCAGATCCG ACCCTTCTCT
GAGCTTCACC
LIF rs737921:
TCCCCCTGGG CTGTGTACTG AGGGGCAGAA GGGAGGTGAC
GTGGGAGTCA GGGTCAGTG TCCCAGCCCT GCCGCCAACC
CTTTGGGCAA GCTCTTGCGT CTGTTTCCCC ATCTAGCGCA
TGAGGACCCA ACTCCTTGCC CTGTAAGCAT CTGGAATTGT
CATGAGAGCC AAAACTAATT
A/G
TAATGTGAGT GCCCTTGCTA AAGATCAAAG ACTGAGCCAT
GCACGCAGTC ATCATTATCA TCATCATCAT CATCACCACC
CTAAGGGGAC AGAGGGGAAA ACTCGGTGTC TAGCCCTAGC
TGGGGCACCA CACACAAGTA CTTCCATCCC TGCACTCACA
ATGTTCCGGG ACGCCCCTCC
LIF rs929273:
CCCTGGTGCC TCACGCCCAT TTCCCCTCCA TCCCTCGCTC
CCTGCAGCAG GACAATCACA AGATAAGAAG TGCCAGGTCC
CCACCTTTGC ACTCAGTTCT CCCCTTGCTA ACTGGGCACC
CTGGGGAAGC TTCCCTGGGG AAGCTTGGGC AGGAAGTGGC
A/G
GGAGTCTGGG GGTGGTLTAA TCAAGCCCTC TCCCCATTCT
CTCCTTCCAG CCCCAAAAGG TCCCCTCAAC CCAGATCAGG
ACAGCCCCTA ATGATATTTA CAAGCCCCCT CCCTGCCATC
TCCTGTCAGT ATCCCAGGGG TAACTTACAT AGAGAATAAA
GAGGGCATTG GCACTGCCAT
LIF rs2267153:
TGAGGCTGGG GAAGGGGCTA GGAAGACATG GGGGTAGGGG
TGACTGACTC AGTTCTGTCG GGACACTCTG GGAAGGTGCT
TCTGGGAAGG CGGTCCAGCA TTTCCATTCT GAAGCAGGAC
TGAGAGAGGC TTGGCGAAAT CGTACCCCAG TTTCCTCCTC
C/G
GGGTGCTGAT TGATGGTTGG GGAAACTGAG AAGTGGCTGG
TCCCTTCCAG ACCTGCCTTG GAAGCCCCTT TGAGCCCAGC
CTCAGAGAAT GATGGAGGTC CCCAAAAAGT GCTTCTAGAG
GCTCTAAGGC AGTGTCACAT GTTCTGGCGT CTTCTGAGGC
CAGGCGATTT GTGAATGAGG
SLC11A2 rs224589:
CTCTTGTACA GTACTCTTGT TTTAGCTTTC GTAAACTCTG
GGCTTTCACC GGACCAGGTT TTCTTATGAG CATTGCCTAC
CTGGATCCAG GAAATATTGA ATCCGATTTG CAGTCTGGAG
CAGTGGCTGG ATTTAAGGTG AACATCTAGT CCTACCCCTG
TCCTTTTAAG CACATAATAC
A/C
CTCTCACATC CTTTTCTCCA CCCTGCATGT TGGATAGTAG
CCTCAGGGGC TACATGCAGA TACTTCATTG GCAGTGGCTC
TTATGTGTAA AGTACTTTCC ATTTGGTCTT ATTTTTATCC
ACATAGTTTC CTTGAACAAAGGAGAAACTA CATATAGGAGA
AACTGAGGCTCAGAAAGGT
HMOX1 rs5755709:
GGGTGATGGA GGCTGCAGTG AGCCGAGATC GTGCCACTGC
ACTCCAGCCT GAGTGACAGAGTGAGACCCC ATCGCAAAAA
AAAAAAAAAA TAAGTCAAGG ATGATGATGA TATAGACTCA
GGGAATATCA TTAAGTGAAC
G/A
AGAAATTATC TTTATTCCCC ACTTTTAACA TGGGGAAACT
GAGGCCCCAG GAAGACAACC AAGTATTGGC TGAATTGAGC
TGAGGGAGAT CTCAAATCAC TCAATAGCGA CCACCACCTT
CCCAGGCAGC TATCGAAGTT CCCATAATGG GCAGATGGAT
CACCTGGGGT CAGGAGTTCG
HMOX1 rs2071748:
AATTTTTTTT TTAATCCTAC TTTCGAGGTG TGTTTGGAGT
TGCTCTCTGC TGAATCTAGA CTCTGGGGC TCTGCCAGCC
TGGGGGAGCA TGCTTGGTTC TCTTGGTGGC ATCTGTCCCT
CACTAGCTAC GGAGGACCTG AGCCAGACAT CACCCTGGCT
A/G
CGGTGTTCCA TGTCTCACAG ATAGCCCAGT TCAGGGAGGC
GACATGCCCA AGAGTGCTCA GTTAGCTGGT GTCAGAACTG
GGCCTTGAAC CTTGGTCTGC CCACCTCCAG GTCTCACTCA
TTCCCTTCTT TCAATAATTT GTTAGTATTT TTTTTTTTAA
CTCCTGGGCT TAAGCATCCT
TMPRSS6 rs855791:
GGCTCCTGAG ATGCAAAGGG AATAATGTTA GGGAGAATAG
AGAACAGGGGCTCCAGGCTC CTGAGATCTC ACTTCTGCCC
TTGACCACGG ACAGGCCCCA TCAGCAACGC TCTGCAGAAA
GTGGATGTGC AGTTGATCCC ACAGGACCTG TGCAGCGAGG
C/T
CTATCGCTAC CAGGTGACGC CACGCATGCT GTGTGCCGGC
TACCGCAAGG GCAAGAAGGA TGCCTGTCAGGTGAGTCCCC
CGGGCATGGG AGGGAGAGAG GAGGGAGAAAGGATGCTGCC
CACATCACCA GGGTCTGGCC CTTTGCTCAC ATCAGCCTGC
TGAAGCCTCC CATCCTCCCA
TMPRSS6 rs733655:
CATAGGCCCA GGAGGCCAAG GTCATGGGTC AGCACCACTA
GGCATCCTTC CACTCGTGAG GTCACCCAGG GATCCCACAG
TGTGTGCTAA CCACCTACTA CATGGGGTAC GCCAGTTAAC
CAAGACAGAT GTGCCTCCCC T
C/T
GTGAAGCTGA CAGTGGTGGG TAAGAAAGGC GTGGCTCTGG
CAACCACACAGCATGTGGCA TCTGTCTGTG GGCAGTGCCA
TCAGGGAGCA GTGCCACATG GTGCTGTTGA GGGGATGTGA
CGAGGACACT CAGCCTGGGC CAGAGTGGAG TGACCCTCCA
GCTGAGATGT GGGATGGGG

TABLE 8
Genotyping Methods for Each Single Nucleotide Polymorphism that Has Predictive Value

| SNP | Genotyping Method | Detail |
|---|---|---|
| IL10 rs1800872 C/A | Taqman allelic discrimination | ABI Cat No C 1747363_10 |
| ACP1 rs12714402 | Taqman allelic discrimination | ABI Cat No C 31126924_10 |
| PKR (EIF2AK2) rs2270414 C/T | Taqman allelic discrimination | ABI Cat No C 15957501_10 |
| PKR (EIF2AK2) rs12712526 A/G | Taqman allelic discrimination | ABI Cat No C 31844699_10 |
| PKR (EIF2AK2) rs2254958 C/T | Taqman allelic discrimination | ABI Cat No C 11162026_20 |
| STEAP3 rs865688 A/G | Taqman allelic discrimination | ABI Cat No C 3255692_10 |
| SLC40A1 rs1439812 T/G | Taqman allelic discrimination | ABI Cat No C 2108632_10 |
| CTLA4 rs231775 A/G | Taqman allelic discrimination | ABI Cat No C 2415786_20 |
| TF rs1049296 C/T | Taqman allelic discrimination | ABI Cat No C 7505275_10 |
| TF rs8649 G/C | Taqman allelic discrimination | ABI Cat No C 148061_10 |
| TFrs1130459 G/A | Taqman allelic discrimination | ABI Cat No C 25647443_10 |
| TF rs4481157 G/A | Taqman allelic discrimination | ABI Cat No C 27915079_10 |
| LTF rs1042073 C/T | Taqman allelic discrimination | ABI Cat No C 2610629_1_{—} |
| EGF rs4444903 A/G | Taqman allelic discrimination | ABI Cat No C 27031637_10 |
| NFKB1 rs4648022 C/T | Taqman allelic discrimination | ABI Cat No C 31213476_10 |
| IRF4 rs12203592 C/T | Taqman allelic discrimination | ABI Cat No C 31918199_10 |
| BMP6 rs17557 G/C | Taqman allelic discrimination | ABI Cat No C 620727_1_{—} |
| EDN1 rs5370 G/T | Taqman allelic discrimination | ABI Cat No C 598677_1_{—} |
| HFE rs807212 C/T | Taqman allelic discrimination | ABI Cat No C 2185346_10 |
| HFE rs1800562 G/A | Taqman allelic discrimination | ABI Cat No C 1085595_10 |
| HIST1H4C rs17596719 G/A | Taqman allelic discrimination | ABI Cat No C 32936064_10 |
| HIST1H1T rs198844 C/G | Taqman allelic discrimination | ABI Cat No C 3266627_10 |
| UBD rs2534790 C/A | Taqman allelic discrimination | ABI Cat No C 11195030_10 |
| HLA-G rs1736939 C/T | Taqman allelic discrimination | ABI Cat No C 26543909_10 |
| HLA-G rs1704 indel | PCR based genotyping | PCR based genotyping |
| ZNRD1 rs9261269 G/A | Taqman allelic discrimination | ABI Cat No C 25960057_10 |
| HLA-E rs1264456 C/T | Taqman allelic discrimination | ABI Cat No C 8942134_10 |
| DDR1 rs1264328 T/C | Taqman allelic discrimination | ABI Cat No C 8941965_10 |
| DDR1 rs1264323 C/T | Taqman allelic discrimination | ABI Cat No C 8941948_10 |
| DDR1 rs1049623 A/G | Taqman allelic discrimination | ABI Cat No C 8941925_1_{—} |
| HLA-C rs9264942 T/C | Taqman allelic discrimination | ABI Cat No C 29901957_10 |
| MICA rs1051792 G/A | PCR-RFLP | HPyCH4III RFLP analysis |
| MICA STR UniSTS:464273 | Fragment Analysis | Fragment analysis |
| BAT3 rs2077102 G/T | Taqman allelic discrimination | ABI Cat No C 2451875_1_{—} |
| HSPA1B rs1061581 A/G | PCR-RFLP | PstI RFLP analysis |
| SKIV2L rs419788 G/A | Taqman allelic discrimination | ABI Cat No C 940302_1_{—} |
| NOTCH4 rs3096702 T/C | Taqman allelic discrimination | ABI Cat No C 27454395_10 |
| BTNL2 rs9268480 C/T | Taqman allelic discrimination | ABI Cat No C 2488470_10 |
| HLA-DRA rs7192 G/T | Taqman allelic discrimination | ABI Cat No C 8848630_20 |
| HLA-DRA rs3135388 C/T | High Resolution Melting | LightScanner |
| HLA-DQA1 rs1142316 A/C | PCR-RFLP | BglII RFLP analysis |
| HLA-DRB1-DQA1 region | Taqman allelic discrimination | ABI Cat No C 16222527_10 |
| rs2395225 T/C | | |
| HLA-DRB1-DQA1 region | Taqman allelic discrimination | ABI Cat No C 29847766_10 |
| rs9271586 T/G | | |
| RXRB rs6531 T/C | Taqman allelic discrimination | ABI Cat No C 8851285_10 |
| RXRB rs2076310 T/C | Taqman allelic discrimination | ABI Cat No C 16167918_10 |
| HSD17B8/RXRB rs365339 G/A | Taqman allelic discrimination | ABI Cat No C 2215080_10 |
| HSD17B8/RXRB rs421446 T/C | Taqman allelic discrimination | ABI Cat No C 27015692_10 |
| DAXX rs2239839 G/T | Taqman allelic discrimination | ABI Cat No C 2479329_20 |
| DAXX rs1059231 T/C | Taqman allelic discrimination | ABI Cat No C 2479328_1_{—} |
| DAXX rs2073524 T/A | Taqman allelic discrimination | ABI Cat No C 2479883_1_{—} |
| VEGFA rs1570360 | Taqman allelic discrimination | ABI Cat No C 1647379_10 |
| IL6 rs1800797 G/A | Taqman allelic discrimination | ABI Cat No C 1839695_20 |
| TFR2 rs10247962 A/G | Taqman allelic discrimination | ABI Cat No C 2184558_10 |
| SLC39A14 rs11136002 | Taqman allelic discrimination | ABI Cat No C 31674398_10 |
| SLC39A4 rs2272662 G/A | Taqman allelic discrimination | ABI Cat No C 26034235_10 |
| LCN2 rs878400 T/C | Taqman allelic discrimination | ABI Cat No C 11886015_10 |
| KLRK1 rs1049174 G/C | Taqman allelic discrimination | ABI Cat No C 9345347_10 |
| KLRK1 rs2617160 A/T | Taqman allelic discrimination | ABI Cat No C 1841959_10 |
| KLRC4 rs2734565 A/G | Taqman allelic discrimination | ABI Cat No C 12110424_10 |
| KLRC4 rs2617170 C/T | Taqman allelic discrimination | ABI Cat No C 1842316_10 |
| KLRC4 rs2617171 C/G | Taqman allelic discrimination | ABI Cat No C 26984346_10 |
| KLRC4 rs1841958 C/A | Taqman allelic discrimination | ABI Cat No C 1842314_10 |
| KLRC1 rs1983526 C/G | Taqman allelic discrimination | ABI Cat No C 11919464_10 |
| SLC11A2 rs224589 C/A | Taqman allelic discrimination | ABI Cat No C 2967992_1_{—} |
| IFNG rs2069727 A/G | Taqman allelic discrimination | ABI Cat No C 2683475_10 |
| HMOX1 rs2071748 G/A | Taqman allelic discrimination | ABI Cat No C 2469922_1_{—} |
| TP53 rs1042522 C/G | Taqman allelic discrimination | ABI Cat No C 2403545_10 |
| LIF rs929271 T/G | Taqman allelic discrimination | ABI Cat No C 7545904_10 |
| LIF rs737921 G/A | Taqman allelic discrimination | ABI Cat No C 2292624_20 |
| LIF rs929273 G/A | Taqman allelic discrimination | ABI Cat No C 2624327_10 |
| LIF rs2267153 C/G | Taqman allelic discrimination | ABI Cat No C 15871704_10 |
| HMOX1 rs5755709 | High Resolution Melting | LightScanner |
| TMPRSS6 rs855791 C/T | Taqman allelic discrimination | ABI Cat No C 3289902_10 |
| TMPRSS6 rs733655 T/C | Taqman allelic discrimination | ABI Cat No C 3289858_1_{—} |

What is claimed is:

1. A method of determining a risk for childhood leukemia in a female, comprising the steps of:
(a) obtaining a biological sample from a female;
(b) isolating nucleic acids from said biological sample; and
(c) performing polymerase chain reaction (PCR) on said isolated nucleic acids to determine the presence of a SNP present in a gene selected from the group consisting of a HLA gene, iron regulatory gene, and cytokine gene, wherein:
(i) at least one SNP selected from the group consisting of BMP6 rs17557, UBD rs2534790, HLA-G rs1736939, HLA-G rs1704, ZNRD1 rs9261269, DDR1 rs1264328, DDR1 rs1264323, DDR1 rs1049623, HLA-C rs9264942, SKIV2L rs419788, HLA-DRA rs3135388, DAXX rs2073524, DAXX rs1059231, and DAXX rs2239839 that is present in said HLA gene, or
(ii) at least one SNP selected from the group consisting of STEAP3 rs865688, SLC40A1 rs1439812, SLC40A1 rs1439812, HFE rs807212, TFR2 rs10247962, LCN2 rs878400, SLC11A2 rs224589, HMOX1 rs2071748, and HMOX1 rs5755709 that is present in said iron regulatory gene, or
(iii) at least one SNP selected from the group consisting of IL6 rs 1800797 and IL10 rs1800872 that is present in said cytokine gene, and
wherein the presence of said SNP present in said gene is indicative of a risk for childhood leukemia in said female.

2. The method of claim 1, wherein the presence of UBD rs2534790, SKIV2L rs419788, HLA-DRA rs3135388, DAXX rs2073524, DAXX rs1059231, DAXX rs2239839, SLC40A1 rs1439812, TFR2 rs10247962, or IL6 rs1800797 is indicative for an increased risk for childhood leukemia in said female.

3. The method of claim 1, wherein the presence of BMP6 rs17557, HLA-G rs1736939, HLA-G rs1704, ZNRD1 rs9261269, DDR1 rs1264328, DDR1 rs1264323, DDR1 rs1049623, HLA-C rs9264942, STEAP3 rs865688, HFE rs807212, LCN2 rs878400, SLC11A2 rs224589, HMOX1 rs2071748, HMOX1 rs5755709, IL10 rs1800872, or SLC40A1 rs1439812 is indicative for a decreased risk for childhood leukemia in said female.

4. The method of claim 1, wherein said SNP includes a combination of HLA-G rs1736939 and HLA-G rs1704 from said HLA gene, and wherein the presence of said combination of SNP is indicative of a decreased risk for childhood leukemia.

5. The method of claim 1, wherein said SNP includes a combination of DDR1 rs1264328, DDR1 rs1264323, and DDR1 rs1049623 from said HLA gene, and wherein the presence of said combination of SNP is indicative of a decreased risk for childhood leukemia.

6. The method of claim 1, wherein said SNP includes a combination of DAXX rs2073524, DAXX rs1059231, and DAXX rs2239839 from said HLA gene, and wherein the presence of said combination of SNP is indicative of an increased risk for childhood leukemia.

7. The method of claim 1, further comprising a SNP selected from the group consisting of EGF rs444-4903, EDN1 rs5370, VEGFA rs1570360, and TP53 rs1042522, wherein the presence of EGF rs444-4903 or EDN1 rs5370 is indicative of a decreased risk for childhood leukemia, and the presence of VEGFA rs1570360 or TP53 rs1042522 is indicative of an increased risk for childhood leukemia.

8. The method of claim 7, wherein said SNP is a combination of at least 4 SNPs selected from the group consisting of DRB, DAXX haplotype, EDN1 rs5370, HMOX1 rs2071748, TFR2 rs10247962, TP53 rs1042522, and IL10 rs1800872, wherein the presence of said combination of the 4 SNPs is indicative of an increased risk for childhood leukemia.

9. The method of claim 8, wherein said SNP is a combination of at least 5 SNPs selected from the group consisting of DRB, DAXX haplotype, EDN1 rs5370, HMOX1 rs2071748, TFR2 rs10247962, TP53 rs1042522, and IL10 rs1800872, wherein the presence of said combination of the 5 SNPs is indicative of an increased risk for childhood leukemia.

10. The method of claim 1, wherein childhood leukemia is childhood acute lymphoblastic leukemia (ALL).

11. The method of claim 1, wherein said biological sample is selected from the group consisting of blood, buccal mucosal cells, skin, hair and tissue.

12. The method of claim 11, wherein said blood is umbilical cord blood.

13. The method of claim 1, wherein said isolating step is performed using phenol-chloroform.

14. The method of claim 1, wherein said nucleic acids are genomic DNA.

15. The method of claim 1, wherein said polymerase chain reaction is performed by TaqMan allelic discrimination assay or PCR-restriction fragment length polymorphism assay.

16. A method of determining a risk for childhood leukemia in a male, comprising the steps of:
(a) obtaining a biological sample from a male;
(b) isolating nucleic acids from said biological sample; and
(c) performing polymerase chain reaction (PCR) on said isolated nucleic acids to determine the presence of a SNP present in a gene selected from the group consisting of a HLA gene, iron regulatory gene, and cytokine gene, wherein:
(i) at least one SNP selected from the group consisting of NFKB1 rs4648022, MICA rs1051792, MICA STR allele 185 bp (A5.1), BAT3 rs2077102, HSPA1B rs1061581, BTNL2 rs9268480, HLA-DRA rs7192, HLA-DQA1 rs1142316, NOTCH4 rs3096702, HLA-DRB1-DQA1 rs2395225, and HLA-DRB1-DQA1 rs9271586 that is present in said HLA gene; or
(ii) at least one SNP selected from the group consisting of TF rs1049296, TF rs8649, TF rs1130459, TF rs4481157, LTF rs1042073, HFE rs807212, SLC39A14 rs11136002, SLC39A4 rs2272662, LCN2 rs878400, TMPRSS6 rs733655, and TMPRSS6 rs855791 that is present in said iron regulatory gene; or
(iii) at least one SNP selected from the group consisting of IL10 rs1800872, PKR rs2270414, PKR rs12712526, PKR rs2254958, CTLA4 rs231775, IRF4 rs12203592, NKG2D rs1049174, NKG2D rs2617160, NKG2D rs2734565, NKG2D rs2617170, NKG2D rs2617171, NKG2D rs1841958, NKG2D rs1983526, and IFNG rs2069727 that is present in said cytokine gene, and
wherein the presence of said SNP present in said gene is indicative of a risk for childhood leukemia in said male.

17. The method of claim 16, wherein the presence of MICA rs 1051792, MICA STR allele 185 bp (A5.1), HSPA1B rs1061581, BTNL2 rs9268480, HLA-DRA rs7192, HLA-DQA1 rs 1142316, NOTCH4 rs3096702, HLA-DRB1-DQA1 rs2395225, and HLA-DRB1-DQA1 rs9271586, SLC39A4 rs2272662, TMPRSS6 rs733655, CTLA4 rs231775, IRF4 rs12203592, NKG2D rs1049174, NKG2D rs2617160, NKG2D rs2734565, NKG2D rs2617170, NKG2D rs2617171, NKG2D rs1841958, or NKG2D rs1983526 is indicative for an increased risk for childhood leukemia in said male.

18. The method of claim 16, wherein the presence of NFKB1 rs4648022, BAT3 rs2077102, HSPA1B rs1061581, TF rs1049296, TF rs8649, TF rs1130459, TF rs4481157, LTF rs1042073, HFE rs807212, SLC39A14 rs11136002, TMPRSS6 rs855791, IL10 rs1800872, PKR rs2270414, PKR rs12712526, PKR rs2254958, IFNG rs2069727, or LCN2 rs878400 is indicative for a decreased risk for childhood leukemia in said male.

19. The method of claim 16, wherein said SNP includes a combination of MICA rs 1051792 and MICA STR allele185 bp (A5.1) from said HLA gene, wherein the presence of said combination is indicative of an increased risk for childhood leukemia.

20. The method of claim 16, wherein said SNP includes a combination of HSPA1B rs1061581, BTNL2 rs9268480, and HLA-DRA rs7192 from said HLA gene, wherein the presence of said combination is indicative of an increased risk for childhood leukemia.

21. The method of claim 16, wherein said SNP includes a combination of HSPA1B rs1061581, HLA-DRA rs7192, and HLA-DQA1 rs1142316 from said HLA gene, wherein the presence of said combination is indicative of an increased risk for childhood leukemia.

22. The method of claim 16, wherein said SNP includes a combination of HLA-DRB1-BQA1 rs2395225 and HLA-DRB1-DQA1 rs9271586 from said HLA gene, wherein the presence of said combination is indicative of an increased risk for childhood leukemia.

23. The method of claim 16, wherein said SNP includes a combination of TF rs1049296, TF rs8649, TF rs1130459, and TF rs4481157 from said iron regulatory gene, wherein the presence of said combination is indicative of a decreased risk for childhood leukemia.

24. The method of claim 16, wherein said SNP includes a combination of PKR rs2270414, PKR rs12712526, and PKR rs2254958 from said iron regulatory gene, wherein the presence of said combination is indicative of a decreased risk for childhood leukemia.

25. The method of claim 16, wherein said SNP includes a combination of NKG2D rs1049174, NKG2D rs2617160, NKG2D rs2734565, NKG2D rs2617170, NKG2D rs2617171, NKG2D rs1841958, and NKG2D rs1983526 from said cytokine gene, wherein the presence of said combination is indicative of an increased risk for childhood leukemia.

26. The method of claim 16, further comprising a SNP selected from the group consisting of ACP1 rs12714402, and TP53 rs1042522, wherein the presence of ACP1 rs12714402 or TP53 rs1042522 is indicative of an increased risk for childhood leukemia.

27. The method of claim 26, wherein said SNP is a combination of at least 4 SNPs selected from the group consisting of DRB1 region, HSPA1 B rs 1061581, MICA haplotype, HFE rs807212, TMPRSS6 rs733655, LTF rs1042073, and PKR haplotype, wherein the presence of said combination of at least 4 SNPs is indicative of an increased risk for childhood leukemia.

28. The method of claim 26, wherein said SNP is a combination of at least 5 SNPs selected from the group consisting of DRB1 region, HSPA1B rs1061581, MICA haplotype, HFE rs807212, TMPRSS6 rs733655, LTF rs1042073, and PKR haplotype, wherein the presence of said combination of at least 5 SNPs is indicative of an increased risk for childhood leukemia.

29. The method of claim 16, wherein childhood leukemia is childhood acute lymphoblastic leukemia (ALL).

30. The method of claim 16, wherein said biological sample is selected from the group consisting of blood, buccal mucosal cells, skin, hair or tissue.

31. The method of claim 30, wherein said blood is umbilical cord blood.

32. The method of claim 16, wherein said isolating step is performed using phenol-chloroform.

33. The method of claim 16, wherein said nucleic acids is genomic DNA.

34. The method of claim 16, wherein polymerase chain reaction is performed by TaqMan allelic discrimination assay or PCR-restriction fragment length polymorphism assay.
